(19) United States
(12) Patent Application Publication
(10) Pub. No.: US 2013/0022912 A1
(43) Pub. Date: Jan. 24, 2013

(54) RADIATION-SENSITIVE RESIN COMPOSITION, METHOD FOR FORMING RESIST PATTERN, POLYMER AND COMPOUND

(71) Applicant: JSR CORPORATION, Tokyo (JP)

(72) Inventors: Mitsuo SATO, Tokyo (JP); Yusuke ANNO, Tokyo (JP); Hiromitsu NAKASHIMA, Tokyo (JP)

(73) Assignee: JSR CORPORATION, Tokyo (JP)

(21) Appl. No.: 13/629,992

(22) Filed: Sep. 28, 2012

Related U.S. Application Data
(63) Continuation of application No. PCT/JP2011/057914, filed on Mar. 29, 2011.

(30) Foreign Application Priority Data
Mar. 31, 2010 (JP) 2010-084713

(51) Int. Cl. C08F 222/18; G03F 7/20; C07C 69/653; G03F 7/004

(52) U.S. Cl. 430/270.1; 430/325; 560/192; 526/242; 526/245

(57) ABSTRACT

A radiation-sensitive resin composition includes a first polymer having a structural unit represented by a following formula (1), and a radiation-sensitive acid generator. R^{C} in the formula (1) preferably represents an aliphatic polycyclic hydrocarbon group having a valency of (n+1) and having 4 to 30 carbon atoms. The structural unit represented by the formula (1) is preferably a structural unit represented by a n following formula (1-1).

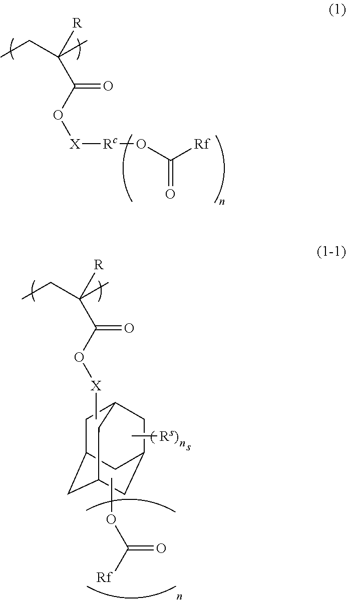

### CROSS-REFERENCE TO RELATED APPLICATIONS

The present application is a continuation application of International Application No. PCT/JP2011/057914, filed Mar. 29, 2011, which claims priority to Japanese Patent Application No. 2010-084713, filed Mar. 31, 2010. The contents of these applications are incorporated herein by reference in their entirety.

### BACKGROUND OF THE INVENTION

1. Field of the Invention

The present invention relates to a radiation-sensitive resin composition, a method for forming a resist pattern, a polymer and a compound.

2. Discussion of the Background

In the field of microfabrication typified by production of integrated circuit devices, fine resist patterns have been conventionally formed by: forming a resist coating film on a substrate with a resin composition containing a polymer having an acid-dissociable group; irradiating the resist coating film through a mask pattern with a radioactive ray having a short wavelength (excimer laser, etc.) to permit exposure; and removing light-exposed sites with an alkaline developer. In this process, a “chemically amplified resist” provided by including in a resin composition a radiation-sensitive acid generating agent that generates an acid upon irradiation with the radioactive ray to improve the sensitivity by the action of the acid has been used.

With respect to such a chemically amplified resist, as a method for forming still finer resist patterns (for example, about 45 nm of line width), utilization of “liquid immersion lithography process” has been increasing. In this method, exposure is carried out in a state in which an exposure light path space (between a lens and a resist coating film) is filled with a liquid immersion medium having a greater refractive index (n) as compared with that of the air or an inert gas (for example, pure water, fluorinated inert liquid, etc.). Therefore, it is advantageous in that even if a numerical aperture (NA) of a lens is increased, the focal depth is less likely to decrease, and higher resolving ability can be achieved.

However, in such a liquid immersion lithography process, a variety of disadvantages may occur when a liquid immersion medium is permeated into the resist coating film. Therefore, for the purpose of preventing such disadvantages, for example, blends of a compound having both an adamantane skeleton and a fluoroacyl group (see PCT International Publication No. 2008/015876), and the like have been studied. On the other hand, bubbles can be included at the interface between a liquid immersion medium and the surface of a resist coating film during scanning exposure, leading to failure in exposure at a predetermined refractive index owing to a lens effect of the bubble, whereby a bubble defect i.e., loss of formability of a pattern having a predetermined shape on the periphery of the bubble, may occur. This bubble defect tends to be more likely to occur as hydrophobicity of the surface of the resist coating film increases. Moreover, in addition to prevention of such a disadvantage, demands for a resin composition used in a liquid immersion lithography process include: suppression of elution of the acid generating agent and the like from the formed resist coating film to the liquid immersion medium, thereby preventing deterioration of performances of the coating film and prevention of contamination of the apparatus such as a lens; and improvement of water draining property of the surface of the resist coating film to prevent leftover of watermarks, thereby enabling exposure by high speed scanning. Known means for accomplishing those may involve a method including forming an upper layer film (protective film) on a resist coating film (see Japanese Unexamined Patent Application, Publication No. 2005-352384). Further, methods for enhancing the hydrophobicity of the surface of the resist coating film have been studied, and for example, a resin composition containing a highly hydrophobic fluorine-containing polymer (see PCT International Publication No. 2007/116664) and the like have been proposed.

When surface wettability for a developer solution and a rinse liquid is deteriorated, removal of development residues deposited during the development on the surface of the resist at sites unexposed with light may be insufficient, whereby development defects such as a blob may occur. For the purpose of preventing such development defects, fluorine-containing polymers that are hydrophobic during liquid immersion lithography but the hydrophobicity decreases upon development with an alkali, specifically, a fluorine-containing polymer including a carboxylic acid into which a fluoroalkyl group has been introduced (see Japanese Unexamined Patent Application, Publication No. 2010-032994), a fluorine-containing polymer including a phenolic hydroxyl group into which a highly hydrophobic fluoroacyl group has been introduced (see Japanese Unexamined Patent Application, Publication No. 2009-139909), and the like have been proposed.

As a marker concerning the aforementioned water draining property as well as efficiency of washing and occurrence of bubble defects which matter in practical liquid immersion lithography process, a dynamic contact angle such as an advancing contact angle or a receding contact angle rather than a static contact angle is believed to be more significant. In addition, for shortening of the time period of a development process, it is also desired to cause the change of the dynamic contact angle within a shorter period of time during a treatment with a developer.

### SUMMARY OF THE INVENTION

According to one aspect of the present invention, a radiation-sensitive resin composition includes a first polymer having a structural unit represented by a following formula (1), and a radiation-sensitive acid generator.

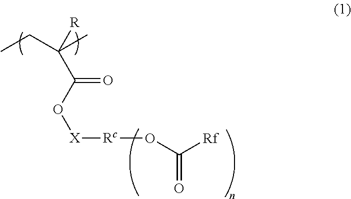

In the formula (1), R represents a hydrogen atom, a methyl group or a trifluoromethyl group. X represents a single bond n or a bivalent linking group. R^{C} represents an aliphatic cyclic hydrocarbon group having a valency of (n+1) and having 3 to 30 carbon atoms, wherein the aliphatic cyclic hydrocarbon group represented by R^{C} is unsubstituted or a part or all of hydrogen atoms included in the aliphatic cyclic hydrocarbon group represented by R^{C} are each substituted. Rf represents a monovalent chain hydrocarbon group having 1 to 30 carbon atoms and having 1 to 10 fluorine atoms, or a monovalent aliphatic cyclic hydrocarbon group having 3 to 30 carbon atoms and having 1 to 10 fluorine atoms. n is an integer of 1 to 3, wherein in a case where n is 2 or 3, Rfs present in plural number are a same or different.

According to another aspect of the present invention, a method for forming a resist pattern includes providing the radiation-sensitive resin composition on a substrate to form a photoresist film. A liquid for immersion lithography is disposed on the photoresist film. The photoresist film is exposed through the liquid for immersion lithography. The exposed photoresist film is developed to form a resist pattern.

According to further aspect of the present invention, a n polymer includes a structural unit represented by a following formula (1).

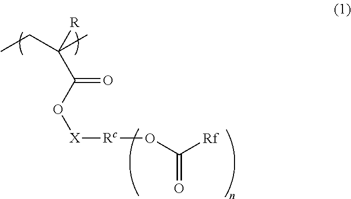

In the formula (1), R represents a hydrogen atom, a methyl group or a trifluoromethyl group. X represents a single bond or a bivalent linking group. R^{C} represents an aliphatic cyclic hydrocarbon group having a valency of (n+1) and having 3 to 30 carbon atoms, wherein the aliphatic cyclic hydrocarbon group represented by R^{C} is unsubstituted or a part or all of hydrogen atoms included in the aliphatic cyclic hydrocarbon group represented by R^{C} are each substituted. Rf represents a monovalent chain hydrocarbon group having 1 to 30 carbon atoms and having 1 to 10 fluorine atoms, or a monovalent aliphatic cyclic hydrocarbon group having 3 to 30 carbon atoms and having 1 to 10 fluorine atoms. n is an integer of 1 to 3, wherein in a case where n is 2 or 3, Rfs present in plural number are a same or different.

According to further aspect of the present invention, a compound is represented by a following formula (1).

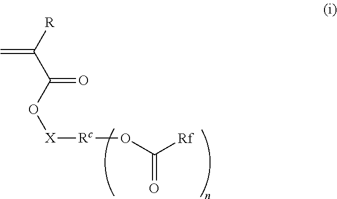

In the formula (1), R represents a hydrogen atom, a methyl group or a trifluoromethyl group. X represents a single bond n or a bivalent linking group. R^{C} represents an aliphatic cyclic hydrocarbon group having a valency of (n+1) and having 3 to 30 carbon atoms, wherein the aliphatic cyclic hydrocarbon group represented by R^{C} is unsubstituted or a part or all of hydrogen atoms included in the aliphatic cyclic hydrocarbon group represented by R^{C} are each substituted. Rf represents a monovalent chain hydrocarbon group having 1 to 30 carbon atoms and having 1 to 10 fluorine atoms, or a monovalent aliphatic cyclic hydrocarbon group having 3 to 30 carbon atoms and having 1 to 10 fluorine atoms. n is an integer of 1 to 3, wherein in a case where n is 2 or 3, Rfs present in plural number are a same or different.

### DESCRIPTION OF THE EMBODIMENTS

An aspect of embodiments of the present invention provides a radiation-sensitive resin composition including

(A) a polymer having a structural unit (I) represented by the following formula (1):

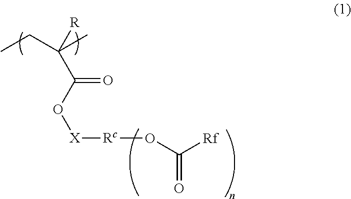

in the formula (1), R represents a hydrogen atom, a methyl group or a trifluoromethyl group; X represents a single bond or a bivalent linking group; R^{C} represents an aliphatic cyclic n hydrocarbon group having a valency of (n+1) and having 3 to 30 carbon atoms, wherein a part or all of hydrogen atoms included in the aliphatic cyclic hydrocarbon group are unsubstituted or substituted; Rf represents a monovalent chain hydrocarbon group having 1 to 30 carbon atoms and having 1 to 10 fluorine atoms, or a monovalent aliphatic cyclic hydrocarbon group having 3 to 30 carbon atoms and having 1 to 10 fluorine atoms; and n is an integer of 1 to 3, and wherein, provided that n is 2 or 3, the Rf present in plural number may be the same or different, and

(B) a radiation-sensitive acid generator.

The radiation-sensitive resin composition contains as the component (A) a polymer having the structural unit (I) represented by the above formula (1) (hereinafter, may be also referred to as “polymer (A)”), and as the component (B) a radiation-sensitive acid generator (hereinafter, may be also referred to as “acid generator (B)”). Since the polymer (A) has the aforementioned specific structural unit (I) that includes a group having a fluorine atom (hereinafter, may be also referred to as “fluorine-containing group”), the distribution thereof on the surface of the coating film is improved resulting from the extent of hydrophobicity thereof, thereby enabling the same to be unevenly distributed on the superficial layer of the coating film. As a result, the surface of the resist coating film will have a great dynamic contact angle without need of separately forming an upper layer film provided for the purpose of shielding the resist coating film from the liquid immersion medium. Therefore, according to the radiation-sensitive resin composition, elution of the acid generating agent and the like from the coating film can be suppressed, and a superior water draining property can be imparted to the surface of the coating film. In addition, since the number of the fluorine atoms of the fluorine-containing group included in the structural unit (I) falls within the above range, the dynamic contact angle of the surface of the coating film is satisfactorily great so as to have a favorable water draining property and highly balanced and controlled so as not to generate bubble defects. Therefore, the radiation-sensitive resin composition can surely inhibit generation of bubble defects.

In addition, since the fluorine-containing group generates a hydroxyl group upon dissociation by hydrolysis in development with an alkali, hydrophobicity of the surface of the resist coating film decreases. As a result, wettability of the surface of the resist coating film with respect to a developer and a rinse liquid is significantly improved after the development with an alkali; therefore, generation of development defects of a resist film that results from inferior efficiency of washing with a rinse liquid can be inhibited. In addition, due to including a bulky aliphatic cyclic hydrocarbon group, the polymer (A) has great hydrophobicity and can minimize soaking water into the film during liquid immersion lithography, whereby defects resulting from liquid immersion such as watermark by soaking of water are inferred to be inhibited. Moreover, according to the polymer (A), the bulky aliphatic cyclic hydrocarbon group is left in the side chain even after the development with an alkali, whereby the polymer remains at the site unexposed to light in the state in which the rigidity is maintained; therefore, such a resist coating film enables superior etching resistance to be obtained in the etching step after the development.

In the above structural unit (I), R^{C} is preferably an aliphatic polycyclic hydrocarbon group having a valency of (n+1) and having 4 to 30 carbon atoms. Accordingly, the rate of hydrolysis in development with an alkali of the polymer (A) is accelerated, thereby further decreasing the dynamic contact angle of the surface of the coating film, and also enabling the etching resistance of the resist coating film to be improved.

The above structural unit (I) is preferably a structural unit (I-1) represented by the following formula (1-1):

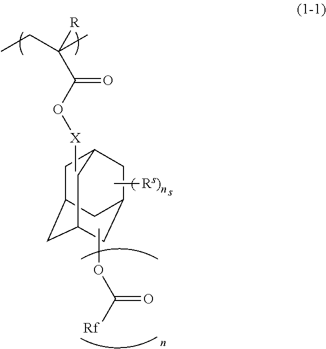

in the formula (1-1), R, X, Rf and n are as defined in connection with the above formula (1); R^{S} represents —R^{P1}, —R^{P2}—O—R^{P1}, —R^{P2}—CO—R^{P1}, —R^{P2}—CO—OR^{P1}, —R^{P2}—O—CO—R^{P1}, —R^{P2}—OH, —R^{P2}—CN or —R^{P2}—COOH; R^{P1} represents a monovalent chain saturated hydrocarbon group having 1 to 10 carbon atoms, a monovalent aliphatic cyclic saturated hydrocarbon group having 3 to 20 carbon atoms or a monovalent aromatic hydrocarbon group having 6 to 30 carbon atoms; R^{P2} represents a single bond, a bivalent chain saturated hydrocarbon group having 1 to 10 carbon atoms, a bivalent aliphatic cyclic saturated hydrocarbon group having 3 to 20 carbon atoms or a bivalent aromatic hydrocarbon group having 6 to 30 carbon atoms; a part or all of hydrogen atoms included in R^{P1} and R^{P2} are unsubstituted or substituted by a fluorine atom; and n_{S} is an integer of 0 to 3.

When the structural unit (I) has a specific structure having the adamantane skeleton described above, the rate of hydrolysis in development with an alkali of the polymer (A) is further accelerated to further decrease the dynamic contact angle of the surface of the coating film, and the etching resistance of the resist coating film is further improved.

As the aforementioned structural unit (I-1), at least one structural unit selected from the group consisting of structural units represented by the following formulae (1-1a), (1-1b) and (1-1c) is particularly preferred.

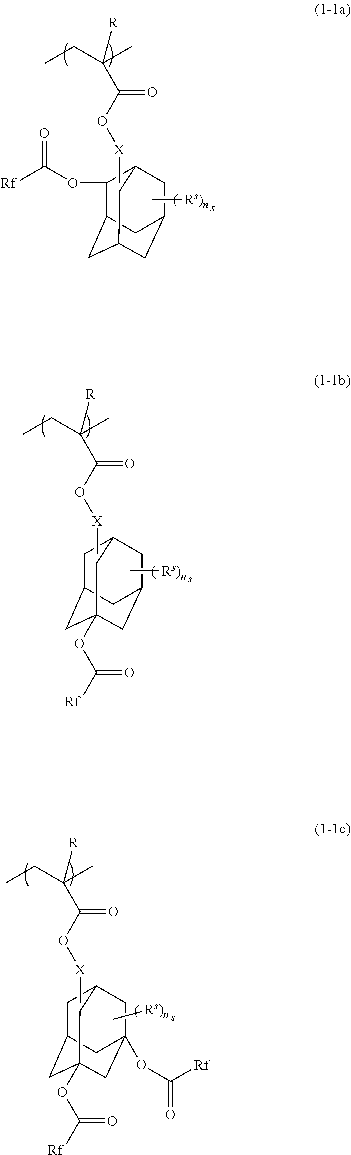

In the formulae (1-1a), (1-1b) and (1-1c), R, X, Rf, R^{S} and n_{S} are as defined in connection with the above formula (1-1).

In the aforementioned structural unit (I), when the fluorine-containing group that is an alkali-dissociable group (i.e., a group that substitutes for a hydrogen atom in a polar functional group, and that is dissociated in the presence of an alkali) binds at the aforementioned specific position of the adamantane structure, the reaction rate of hydrolysis in the development with an alkali is markedly improved, whereby the dynamic contact angle of the surface of the coating film is further decreased.

It is preferred that the radiation-sensitive resin composition further contains (C) a polymer having a content of fluorine atoms less than that of the polymer (A) (hereinafter, may be also referred to as “polymer (C)”), and the polymer (C) has an acid-dissociable group. Due to containing such a polymer (C), the extent of uneven distribution of the polymer (A) on the surface of the resist film increases when a resist film is formed from a composition containing the polymer (A) and the polymer (C). As a result, the aforementioned hydrophobicity and properties of the polymer (A) that result from its decrease can be more efficiently exhibited.

In the radiation-sensitive resin composition, it is preferred that the polymer (A) further has at least one structural unit selected from the group consisting of a structural unit (II) represented by the following formula (2) and a structural unit (III) represented by the following formula (3).

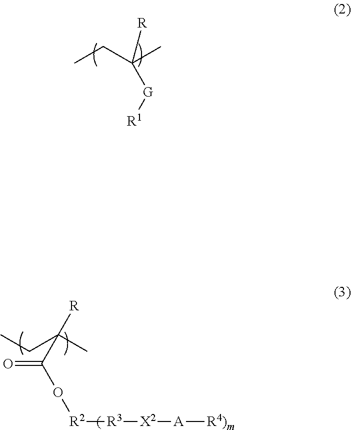

In the formulae (2) and (3), R represents a hydrogen atom, a methyl group or a trifluoromethyl group.
In the formula (2), G represents a single bond, an oxygen atom, a sulfur atom, —CO—O—, —SO₂—O—NH—, —CO—NH— or —O—CO—NH—; and R⁴ represents a monovalent chain hydrocarbon group having 1 to 6 carbon atoms and having at least one fluorine atom or a monovalent aliphatic cyclic hydrocarbon group having 4 to 20 carbon atoms and having at least one fluorine atom.

In the formula (3), R² represents a hydrocarbon group having 1 to 20 carbon atoms and having a valency of (m+1), and a structure in which an oxygen atom, a sulfur atom, —NR′—, carbonyl group, —CO—O— or —CO—NH— is bonded to the end of R² on the side of R³ is also included; R′ represents a hydrogen atom or a monovalent organic group; R³ represents a single bond, a bivalent chain hydrocarbon group having 1 to 10 carbon atoms or a bivalent aliphatic cyclic hydrocarbon group having 4 to 20 carbon atoms; X² represents a bivalent chain hydrocarbon group having 1 to 20 carbon atoms and having at least one fluorine atom; A represents an oxygen atom, —NR″—, —CO—O—* or —SO₂—O—*; R″ represents a hydrogen atom or a monovalent organic group; * denotes a binding site that binds to R⁴; R⁴ represents a hydrogen atom or a monovalent organic group; and m is an integer of 1 to 3, wherein, provided that m is 2 or 3, the R³, X², A and R⁴ present in plural number may be each the same or different.

When the polymer (A) further has at least one structural units selected from the group consisting of the structural unit (II) and the structural unit (III), the degree of change of the dynamic contact angle in the development process of the resist coating film formed from the radiation-sensitive resin composition can be further increased.

The method for forming a resist pattern of the embodiment of the present invention includes the steps of:

(1) forming a photoresist film on a substrate using the radiation-sensitive resin composition described above;

(2) subjecting the photoresist film to liquid immersion lithography through a liquid for immersion lithography which had been placed on the photoresist film; and

(3) forming a resist pattern by developing the photoresist film subjected to the liquid immersion lithography.

Since, the radiation-sensitive resin composition is used in the formation method as a photoresist composition, the surface of the coating film has a superior water breaking property, and the process time can be shortened owing to high speed scanning exposure. In addition, generation of bubble defects and development defects can be inhibited, whereby a favorable resist pattern can be efficiently formed.

The polymer of the embodiment of the present invention has a structural unit (I) represented by the following formula (1):

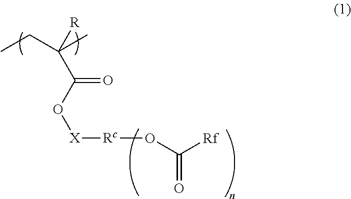

in the formula (1), R represents a hydrogen atom, a methyl group or a trifluoromethyl group; X represents a single bond or a bivalent linking group; R^{C} represents an aliphatic cyclic hydrocarbon group having a valency of (n+1) and having 3 to n 30 carbon atoms, wherein a part or all of hydrogen atoms included in the aliphatic cyclic hydrocarbon group are unsubstituted or substituted; Rf represents a monovalent chain hydrocarbon group having 1 to 30 carbon atoms and having 1 to 10 fluorine atoms, or a monovalent aliphatic cyclic hydrocarbon group having 3 to 30 carbon atoms and having 1 to 10 fluorine atoms; and n is an integer of 1 to 3, wherein, provided that n is 2 or 3, the Rf present in plural number may be the same or different.)

Moreover, it is preferred that the polymer further has at least one structural unit selected from the group consisting of a structural unit (II) represented by the following formula (2) and a structural unit (III) represented by the following formula (3).

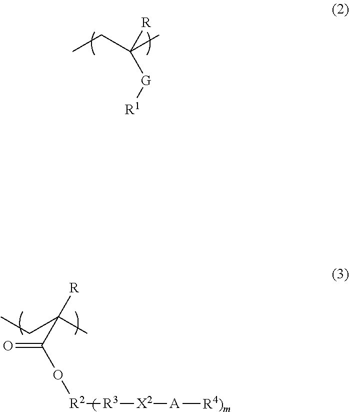

In the formulae (2) and (3), R represents a hydrogen atom, a methyl group or a trifluoromethyl group.
In the formula (2), G represents a single bond, an oxygen atom, a sulfur atom, —CO—O—, —SO₂—O—NH—, —CO—NH— or —O—CO—NH—; R¹ represents a monovalent chain hydrocarbon group having 1 to 6 carbon atoms and having at least one fluorine atom or a monovalent aliphatic cyclic hydrocarbon group having 4 to 20 carbon atoms and having at least one fluorine atom.

In the formula (3), R² represents a hydrocarbon group having 1 to 20 carbon atoms and having a valency of (m+1), and a structure in which an oxygen atom, a sulfur atom, —NR′—, carbonyl group, —CO—O— or —CO—NH— is bonded to the end of R² on the side of R³ is also included; R′ represents a hydrogen atom or a monovalent organic group; R³ represents a single bond, a bivalent chain hydrocarbon group having 1 to 10 carbon atoms or a bivalent aliphatic cyclic hydrocarbon group having 4 to 20 carbon atoms; X² represents a bivalent chain hydrocarbon group having 1 to 20 carbon atoms and having at least one fluorine atom; A represents an oxygen atom, —NR″—, —CO—O—* or —SO₂—O—*; R″ represents a hydrogen atom or a monovalent organic group; * denotes a binding site that binds to R⁴; R⁴ represents a hydrogen atom or a monovalent organic group; and m is an integer of 1 to 3, wherein, provided that m is 2 or 3, the R³, X², A and R⁴ present in plural number may be each the same or different.

The polymer has the aforementioned structural unit (I), and also may further have at least one structural units selected from the group consisting of the structural unit (II) and the structural unit (III). Such a polymer is characterized by having high hydrophobicity, whereas having decreased hydrophobicity due to hydrolysis; therefore, for example, the dynamic contact angle of the surface of the resist coating film can be controlled to become high during the exposure, and low after the development with an alkali. Therefore, the polymer is suitable for radiation-sensitive resin compositions and the like used in, for example, lithography techniques.

The compound of the embodiment of the present invention is represented by the following formula (1):

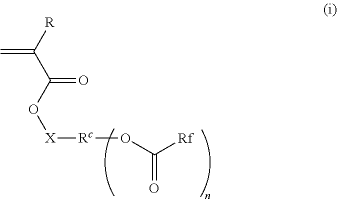

in the formula (1), R represents a hydrogen atom, a methyl group or a trifluoromethyl group; X represents a single bond or a bivalent linking group; R^{C} represents an aliphatic cyclic hydrocarbon group having a valency of (n+1) and having 3 to 30 carbon atoms, wherein a part or all of hydrogen atoms included in the aliphatic cyclic hydrocarbon group are unsubstituted or substituted; Rf represents a monovalent chain hydrocarbon group having 1 to 30 carbon atoms and having 1 to 10 fluorine atoms, or a monovalent aliphatic cyclic hydrocarbon group having 3 to 30 carbon atoms and having 1 to 10 fluorine atoms; and n is an integer of 1 to 3, wherein, provided that n is 2 or 3, the Rf present in plural number may be the same or different.

Since the compound of the embodiment of the present invention has a structure represented by the above formula (1), it can be suitably used as a monomer for incorporating the structural unit (I) into the polymer.

Herein, a “hydrocarbon group” as merely referred to includes a chain hydrocarbon group, an aliphatic cyclic hydrocarbon group, and an aromatic hydrocarbon group. This “hydrocarbon group” may be either a saturated hydrocarbon group, or an unsaturated hydrocarbon group.

Also, the “chain hydrocarbon group” means a hydrocarbon group constituted with only a chain structure without including a ring structure in the main chain, and a linear hydrocarbon group and a branched hydrocarbon group are both included. The “aliphatic cyclic hydrocarbon group” means a hydrocarbon group that includes as a ring structure not an aromatic ring structure but only a structure of an aliphatic cyclic hydrocarbon. However, it is not necessary to be constituted with only a structure of an aliphatic cyclic hydrocarbon, and a part thereof may include a chain structure. The “aromatic hydrocarbon group” means a hydrocarbon group that includes an aromatic ring structure as a ring structure. However, it is not necessary to be constituted with only an aromatic ring structure, and a part thereof may include a chain structure or a structure of an aliphatic cyclic hydrocarbon.

As described in the foregoing, since the radiation-sensitive resin composition of the embodiment of the present invention contains a polymer having a specific structural unit and a radiation-sensitive acid generator, the resist coating film formed in a liquid immersion lithography process has an adequately great dynamic contact angle in exposure, whereas the dynamic contact angle significantly decreased in development, whereby the developer favorably spreads in the development with an alkali, and high affinity to the rinse liquid is provided after coating the developer, whereby the rinse liquid favorably spreads, leading to superior developability achieved. As a result, according to the radiation-sensitive resin composition, in addition to suppression of elution from the resist coating film, due to the surface of the coating film having a superior water breaking property, high speed scanning exposure is enabled, and occurrence of various types of defects such as watermark n defects, bubble defects and development defects is inhibited. Accordingly, a favorable resist pattern can be formed.

The embodiments will now be described in detail.

The radiation-sensitive resin composition of the embodiment of the present invention contains (A) a polymer and (B) an acid generator (B), and may contain (C) a polymer as a suitable optional component. Additionally, as other optional components, (D) an acid diffusion controller, (E) a solvent, (F) an additive, and the like may be contained. Hereinafter, each constitutive component will be explained in this order.

### <(A) Polymer>

The polymer (A) in the embodiment of the present invention is a polymer having the structural unit (I) represented by the above formula (1). Since the polymer (A) has a fluorine-substituted hydrocarbon group, it has high hydrophobicity, and when a resist coating film is formed together with other polymer, the distribution of the polymer (A) is improved on the surface thereof, in other words, the polymer (A) tends to be unevenly distributed on the superficial layer of the coating film. As a result, since the formed resist coating film has a great dynamic contact angle, elution of the acid generator and the like from the coating film can be inhibited, and the surface of the coating film attains a superior water draining property. Accordingly, for a similar purpose, a necessity of separately forming an upper layer film for shielding the surface of the resist coating film from the liquid immersion medium is obviated.

On the other hand, since the fluorine-containing group of the polymer (A) generates a hydroxyl group upon dissociation by hydrolysis in development with an alkali, hydrophobicity of the surface of the resist coating film decreases. As a result, wettability of the surface of the coating film with respect to a developer and a rinse liquid is significantly improved after the development with an alkali; therefore, generation of development defects of a resist film that results from inferior efficiency of washing with a rinse liquid can be inhibited. In addition, it is inferred that due to including a bulky aliphatic cyclic hydrocarbon group the polymer (A) has great hydrophobicity and can minimize soaking of water into the film during liquid immersion lithography, whereby defects resulting from liquid immersion such as a watermark by soaking of water are inhibited.

### [Structure Unit (I)]

The structural unit (I) is a structural unit represented by the above formula (1).

In the above formula (1), R represents a hydrogen atom, a methyl group or a trifluoromethyl group; X represents a single bond or a bivalent linking group; R^{C} represents an aliphatic cyclic hydrocarbon group having a valency of (n+1) and having 3 to 30 carbon atoms, wherein a part or all of hydrogen atoms included in the aliphatic cyclic hydrocarbon group are unsubstituted or substituted; Rf represents a monovalent chain hydrocarbon group having 1 to 30 carbon atoms and having 1 to 10 fluorine atoms, or a monovalent aliphatic cyclic hydrocarbon group having 3 to 30 carbon atoms and having 1 to 10 fluorine atoms; and n is an integer of 1 to 3, wherein, provided that n is 2 or 3, the Rf present in plural number may be the same or different.

The bivalent linking group represented by X described above is exemplified by a bivalent chain hydrocarbon group having 1 to 30 carbon atoms, an aliphatic cyclic hydrocarbon group having 3 to 30 carbon atoms bivalent, a bivalent aromatic hydrocarbon group having 6 to 30 carbon atoms, or a bivalent group of the same combined with an ether group, an ester group, a carbonyl group, an imino group, or an amide group. In addition, the bivalent linking group has or does not have a substituent.

Specific examples of the bivalent chain hydrocarbon group having 1 to 30 carbon atoms include: chain saturated hydrocarbon groups such as a methanediyl group, an ethanediyl group, a propanediyl group, a butanediyl group, a pentanediyl group, a hexanediyl group, an octanediyl group, a decanediyl group, an undecanediyl group, a hexadecanediyl group and an icosanediyl group;

chain unsaturated hydrocarbon groups such as an ethenediyl group, a propenediyl group, a butenediyl group, a pentenediyl group, a hexenediyl group, an octenediyl group, a decenediyl group, an undecenediyl group, a hexadecenediyl n group, an icosenediyl group, and alkynediyl groups such as an ethynediyl group, a propynediyl group, a butynediyl group and an octynediyl group, a butadienediyl group, a hexadienediyl group, an octatrienediyl group, and the like.

Specific examples of the bivalent aliphatic cyclic hydrocarbon group having 3 to 30 carbon atoms include:

monocyclic saturated hydrocarbon groups such as a cyclopropanediyl group, a cyclobutanediyl group, a cyclopentanediyl group, a cyclohexanediyl group, a cycloheptanediyl group, a cyclooctanediyl group, a cyclodecanediyl group, a methylcyclohexanediyl group and an ethylcyclohexanediyl group;

monocyclic unsaturated hydrocarbon groups such as a cyclobutenediyl group, a cyclopentenediyl group, a cyclohexenediyl group, a cycloheptenediyl group, a cyclooctenediyl group, a cyclodecenediyl group, a cyclopentadienediyl group, a cyclohexadienediyl group, a cyclooctadienediyl group and a cyclodecadienediyl group; polycyclic saturated hydrocarbon groups such as a bicyclo[2.2.1]heptanediyl group, a bicyclo[2.2.2]octanediyl group, a tricyclo[5.2.1.0^{2,6}]decanediyl group, a tricyclo[3.3.1.1^{3,7}]decanediyl group, a tetracyclo[6.2.1.1^{3,6.}0^{2,7}]dodecanediyl group and an adamantanediyl group;

polycyclic unsaturated hydrocarbon groups such as a bicyclo[2.2.1]heptenediyl group, a bicyclo[2.2.2]octenediyl group, a tricyclo[5.2.1.0^{2,6}]decenediyl group, a n tricyclo[3.3.1.1^{3,7}]decenediyl group and a tetracyclo[6.2.1.1^{3,6.}0^{2,7}]dodecenediyl group, and the like.

Specific examples of the bivalent aromatic hydrocarbon group having 6 to 30 carbon atoms include a phenylene group, a biphenylene group, a terphenylene group, a benzylene group, a phenyleneethylene group, a phenylenecyclohexylene group, a naphthylene group, and the like.

Additionally, specific examples of the bivalent linking group also include groups represented by the following formulae (X-1) to (X-6):

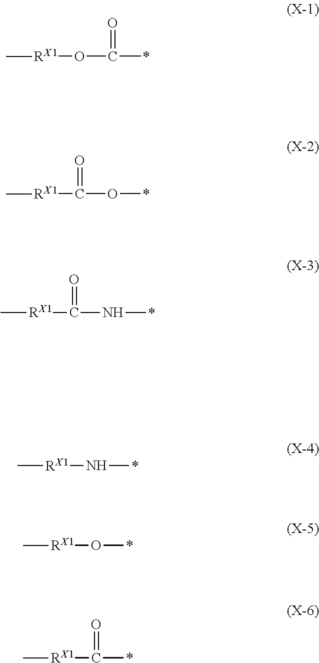

in the above formulae (X-1) to (X-6), R^{x1} each independently represents a bivalent chain hydrocarbon group having 1 to 30 carbon atoms, an aliphatic cyclic hydrocarbon group having 3 to 30 carbon atoms bivalent or a bivalent aromatic hydrocarbon group having 6 to 30 carbon atoms; and * denotes a binding site that binds to R^{C} in the above formula (1).

Specific examples of the aliphatic cyclic hydrocarbon group having a valency of (n+1) and having 3 to 30 carbon atoms represented by the R^{C} described above include:

monocyclic saturated hydrocarbons such as cyclopropane, cyclobutane, cyclopentane, cyclohexane, cycloheptane, cyclooctane, cyclodecane, methylcyclohexane and ethylcyclohexane;

monocyclic unsaturated hydrocarbons such as cyclobutene, cyclopentene, cyclohexene, cycloheptene, cyclooctene, cyclodecene, cyclopentadiene, cyclohexadiene, cyclooctadiene and cyclodecadiene;

polycyclic saturated hydrocarbons such as bicyclo[2.2.1]heptane, bicyclo[2.2.2]octane, tricyclo[5.2.1.0^{2,6}]decane, tricyclo[3.3.1.1^{3,7}]decane, tetracyclo[6.2.1.1^{3,6.}0^{2,7}]dodecane and adamantane;

groups derived by eliminating (n+1) hydrogen atoms from a cyclic hydrocarbon having 3 to 30 carbon atoms or the like such as a polycyclic unsaturated hydrocarbon such as bicyclo[2.2.1]heptene, bicyclo[2.2.2]octene, tricyclo[5.2.1.0^{2,6}]decene, tricyclo[3.3.1.1^{3,7}]decene or tetracyclo[6.2.1.1^{3,6.}0^{2,7}]dodecene, and the like. Among these, since the rate of hydrolysis of the fluorine-containing group that is an alkali-dissociable group is accelerated and the etching resistance of the resist coating film formed is improved, an aliphatic polycyclic hydrocarbon group having a valency of (n+1) and having 4 to 30 carbon atoms is preferred, a bivalent or trivalent aliphatic polycyclic hydrocarbon group having 6 to 15 carbon atoms is more preferred, and a bivalent aliphatic polycyclic hydrocarbon group having 8 to 12 carbon atoms is particularly preferred.

In addition, R^{C} has or does not have a substituent. Examples of such a substituent include —R^{P1}, —R^{P2}—O—R^{P1}, —R^{P2}—CO—R^{P1}, —R^{P2}—CO—OR^{P1}, —R^{P2}—O—CO—R^{P1}, —R^{P2}—OH, —R^{P2}—CN or —R^{P2}—COOH, and the like. Wherein, R^{P1} represents a monovalent chain saturated hydrocarbon group having 1 to 10 carbon atoms, a monovalent aliphatic cyclic saturated hydrocarbon group having 3 to 20 carbon atoms or a monovalent aromatic hydrocarbon group having 6 to 30 carbon atoms; R^{P2} represents a single bond, a bivalent chain saturated hydrocarbon group having 1 to 10 carbon atoms, a bivalent aliphatic cyclic saturated hydrocarbon group having 3 to 20 carbon atoms or a bivalent aromatic hydrocarbon group having 6 to 30 carbon atoms; a part or all of hydrogen atoms included in R^{P1} and R^{P2} is unsubstituted or substituted by a fluorine atom; and R^{C} may have one or more of only one type among the substituents, or each one or more of a plurality of types among the substituents.

In the group represented by Rf described above, it is important that the number of fluorine atoms included in the hydrocarbon group is 1 to 10. When the number of fluorine atoms included in the group represented by Rf falls within the above range, an appropriate extent of the hydrophobicity of the polymer (A) can be attained, whereby the dynamic contact angle of the surface of the resist coating film formed from the radiation-sensitive resin composition becomes high enough for providing a favorable water breaking property, and the dynamic contact angle can be controlled to be highly balanced to an extent that generation of bubble defects can be inhibited. As a result, generation of bubble defects can be surely inhibited. When the number of fluorine atoms included in the group represented by Rf exceeds 10, the polymer (A) has too high hydrophobicity, leading to excessive increase of the advancing contact angle of the surface of the resist coating film formed, whereby bubble defects are more likely to occur. In addition, when the group represented by Rf does not include a fluorine atom, the polymer (A) has insufficient hydrophobicity, whereby a low receding contact angle of the surface of the resist coating film formed is attained, accompanied by deterioration of hydrolysability of the group represented by Rf, and decrease of the hydrophobicity upon development with an alkali is also lessened. The upper limit of the fluorine atoms included in the group represented by Rf is preferably 9, more preferably 8, and still more preferably 7. On the other hand, the lower limit of the number of the fluorine atoms is preferably 2, more preferably 3, and still more preferably 5.

The monovalent chain hydrocarbon group having 1 to 30 carbon atoms and having 1 to 10 fluorine atom represented by Rf described above is exemplified by those derived by substituting 1 to 10 hydrogen atoms of a chain hydrocarbon group having 1 to 30 carbon atoms by a fluorine atom. Examples of the chain hydrocarbon group include a methyl group, an ethyl group, a 1-propyl group, a 2-propyl group, a 1-butyl group, a 2-butyl group, a 2-(2-methylpropyl) group, a 1-pentyl group, a 2-pentyl group, a 3-pentyl group, a 1-(2-methylbutyl) group, a 1-(3-methylbutyl) group, a 2-(2-methylbutyl) group, a 2-(3-methylbutyl) group, a neopentyl group, a 1-hexyl group, a 2-hexyl group, a 3-hexyl group, a 1-(2-methylpentyl) group, a 1-(3-methylpentyl) group, a 1-(4-methylpentyl) group, a 2-(2-methylpentyl) group, a 2-(3-methylpentyl) group, a 2-(4-methylpentyl) group, a 3-(2-methylpentyl) group, a 3-(3-methylpentyl) group, an octyl group, a nonyl group, a decyl group, a dodecyl group, a tetradecyl group, a hexadecyl group, an icosanyl group, and the like.

The monovalent aliphatic cyclic hydrocarbon group having 3 to 30 carbon atoms and having 1 to 10 fluorine atoms represented by the Rf described above is exemplified by those derived by substituting 1 to 10 hydrogen atoms of an aliphatic cyclic hydrocarbon group having 3 to 30 carbon atoms by a fluorine atom. Examples of the aliphatic cyclic hydrocarbon group include a cyclopentyl group, a cyclopentylmethyl group, a 1-(1-cyclopentylethyl) group, a 1-(2-cyclopentylethyl) n group, a cyclohexyl group, a cyclohexylmethyl group, a 1-(1-cyclohexylethyl) group, a 1-(2-cyclohexylethyl group), a cycloheptyl group, a cycloheptyl methyl group, a 1-(1-cycloheptyl ethyl) group, a 1-(2-cycloheptyl ethyl) group, a 2-norbornyl group, a 1-adamantyl group, a 2-adamantyl group, and the like.

Among these, in light of a great dynamic contact angle of the surface of the resist coating film formed before the development, a perfluoroalkyl group having 1 to 4 carbon atoms, a monoperfluoroalkylmethylene group having 2 to 5 carbon atoms or a diperfluoroalkylmethylene group having 3 to 5 carbon atoms is preferred as the group represented by Rf, and of these, a trifluoromethyl group or a perfluoropropyl group is particularly preferred.

Specific examples of preferable structural unit (I) described above include structural units represented by the following formulae (1-1) to (1-4):

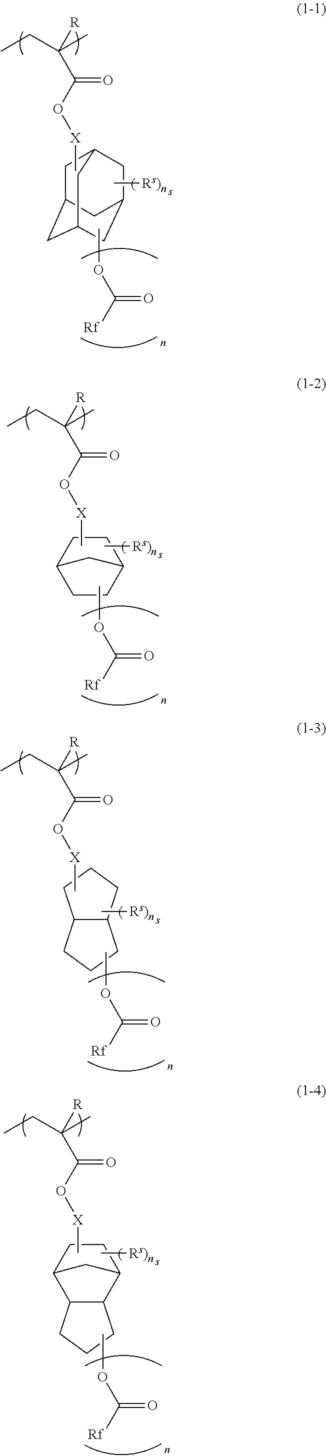

in the above formulae (1-1) to (1-4), R, X, Rf and n are as defined in connection with the above formula (1); R^{S} represents —R^{P1}, —R^{P2}—O—R^{P1}, —R^{P2}—CO—R^{P1}, —R^{P2}—CO—OR^{P1}, —R^{P2}—O—CO—R^{P1}, —R^{P2}—OH, —R^{P2}—CN or —R^{P2}—COOH; R^{P1} represents a monovalent chain saturated hydrocarbon group having 1 to 10 carbon atoms, a monovalent aliphatic cyclic saturated hydrocarbon group n having 3 to 20 carbon atoms or a monovalent aromatic hydrocarbon group having 6 to 30 carbon atoms; R^{P2} represents a single bond, a bivalent chain saturated hydrocarbon group having 1 to 10 carbon atoms, a bivalent aliphatic cyclic saturated hydrocarbon group having 3 to 20 carbon atoms or a bivalent aromatic hydrocarbon group having 6 to 30 carbon atoms, wherein a part or all of hydrogen atoms included in R^{P1} and R^{P2} are unsubstituted or substituted by a fluorine atom; and n_{S} is an integer of 0 to 3.

In the structural unit represented by the above formulae (1-1) to (1-4), the aliphatic cyclic hydrocarbon group having an adamantane skeleton, a norbornane skeleton, a bicyclooctane skeleton or a tricyclodecane skeleton may include any one exemplified as the substituent of R^{C} described above, as a substituent R^{s} that substitutes for a hydrogen atom on the skeleton.

Among these, the structural unit represented by the above formula (1-1) is preferred. Since a fluorine-containing group that is an alkali-dissociable group is bound to a bulky adamantane skeleton due to having such a structural unit, according to the resist coating film formed from the radiation-sensitive resin composition, a high rate of hydrolysis of the group is achieved, with a greater decrease of the dynamic contact angle after the development with an alkali, and the etching resistance of the resist coating film formed is improved. In addition, the structural unit represented by the above formulae (1-1a), (1-1b) and (1-1c) is particularly preferred of these, in light of markedly increased rate of hydrolysis and still further decreased dynamic contact angle after the development with an alkali since the alkali-dissociable group is bound at a specific position of the adamantane structure.

In the above formulae (1-1a), (1-1b) and (1-1c), R, X, Rf, R^{s} and n_{S} are as defined in connection with the above formula (1-1).

In the structural unit represented by the above formula (1-1a), X represents preferably a single bond or an alkanediyl group having 1 to 5 carbon atoms, and more preferably an alkanediyl group having 1 to 5 carbon atoms. When X represents an alkanediyl group having 1 to 5 carbon atoms, the alkali-dissociable group is spaced away from the main chain of the n polymer (A) by a certain distance; therefore, hydrolysis by an alkaline developer is likely to occur, and the rate further increases, whereby decrease of the dynamic contact angle is further enhanced. X may have a hydroxyl group. Specific examples of the structural unit represented by the above formula (1-1a) include those represented by the following formulae (1-1a-1) to (1-1a-9):

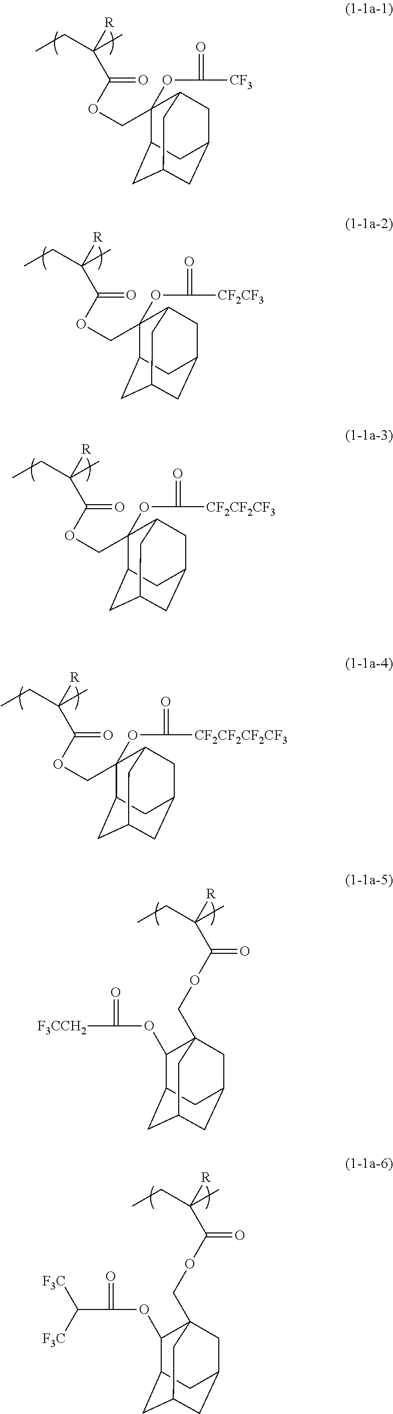

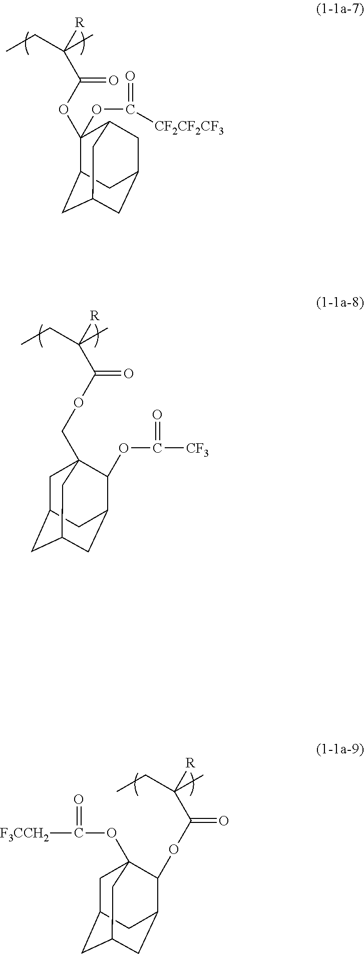

in the above formulae (1-1a-1) to (1-1a-9), R represents a hydrogen atom, a methyl group or a trifluoromethyl group.

Moreover, in the above formulae (1-1b) and (1-1c), X represents preferably a single bond, an alkanediyl group having 1 to 5 carbon atoms, an alkanediyloxy group having 1 to 5 carbon atoms or an alkanediylcarbonyloxy group having 1 to 5 carbon atoms for reasons similar to those in the case of the formula (1-1a) described above. When X is other than a single bond, a hydroxyl group may be included. Specific examples of the structural unit represented by the above formula (1-1b) include those represented by the following formulae (1-1b-1) to (1-1b-9). Also, specific examples of the structural unit represented by the above formula (1-1c) include those represented by the following formulae (1-1c-1) to (1-1c-6).

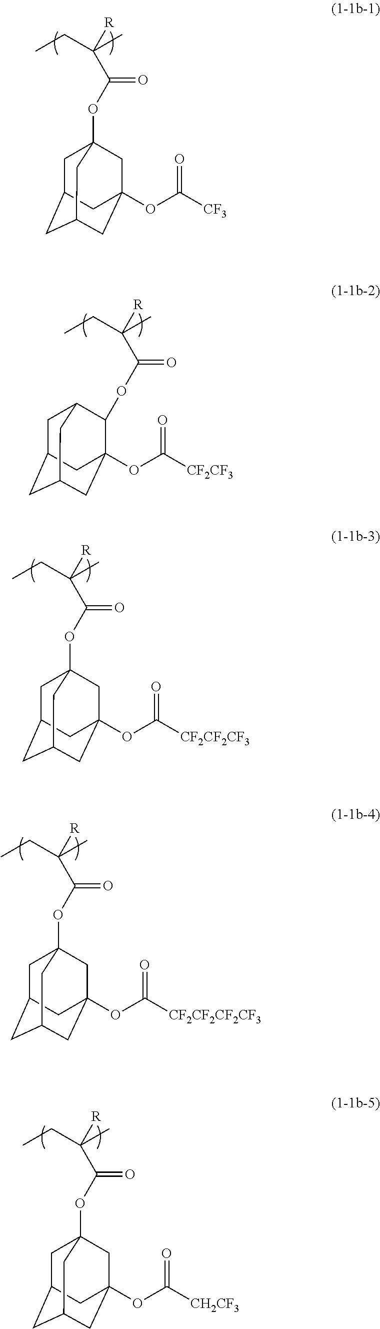

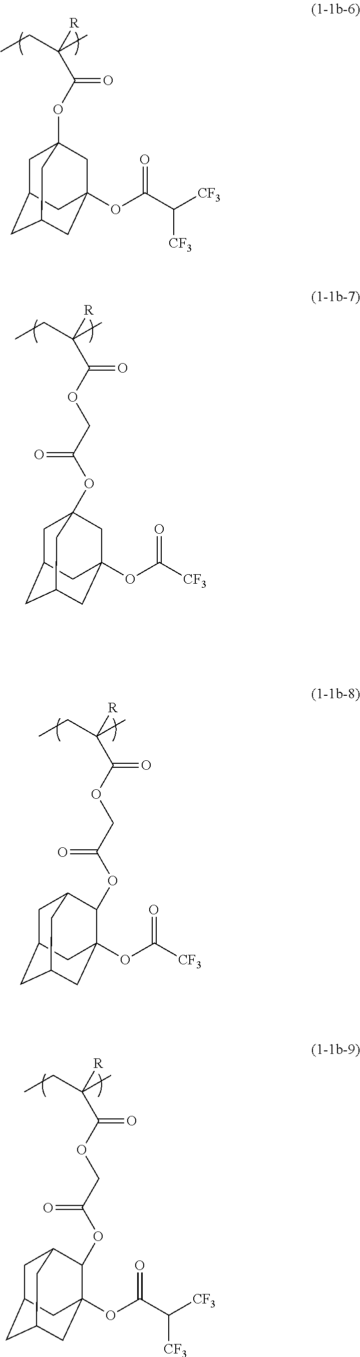

In the above formulae (1-1b-1) to (1-1b-9), R represents a hydrogen atom, a methyl group or a trifluoromethyl group.

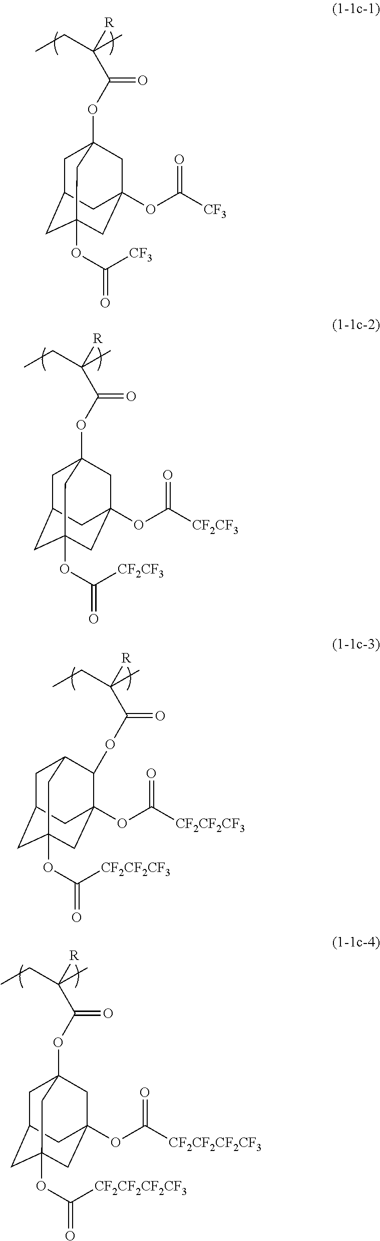

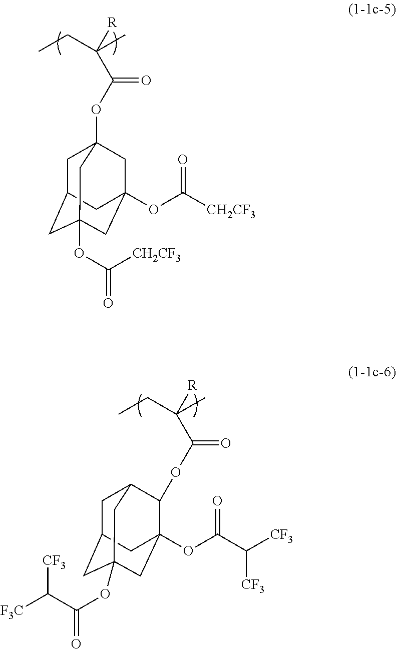

In the above formulae (1-1c-1) to (1-1c-6), R represents a hydrogen atom, a methyl group or a trifluoromethyl group.

Specific examples of the structural unit represented by the above formulae (1-2), (1-3) and (1-4) include groups represented by the following formulae (1-2a), (1-2b), (1-3a) and (1-4a). The polymer (A) may have the structural unit (I) either one type alone, or two types or more in combination thereof.

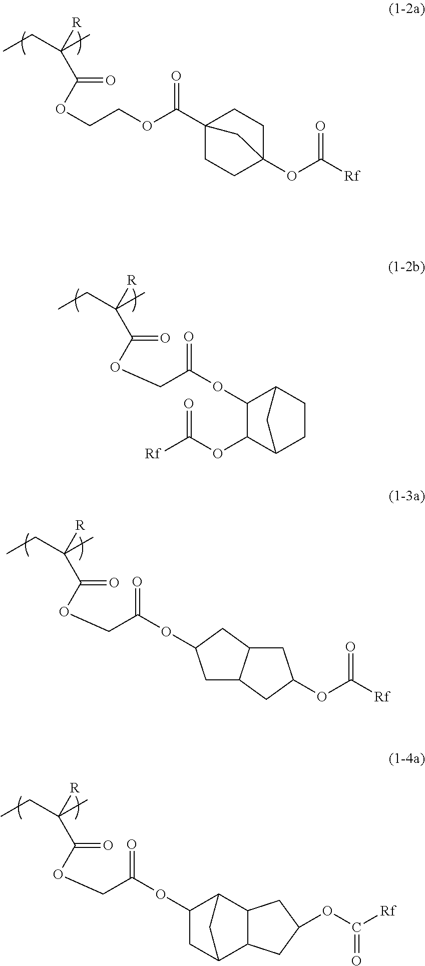

In the above formula (1-2a), (1-2b), (1-3a) and (1-4a), R and Rf are as defined in connection with the above formula (1).

The content of the structural unit (I) with respect to the entire structural units constituting the polymer (A) is preferably 1 to 100 mol %, more preferably 1 to 80 mol %, and n still more preferably 1 to 50 mol %. When the content falls within such a range, a great dynamic contact angle in liquid immersion lithography, as well as enough decrease of the dynamic contact angle by way of the development can be achieved.

The polymer (A) is obtained by radical polymerization of the monomer that gives the structural unit (I), with if necessary a monomer that gives other structural unit, as described later. The method for synthesizing a compound (I) that gives the structural unit (I) is as follows, and the compound (i) can be synthesized according to the following scheme:

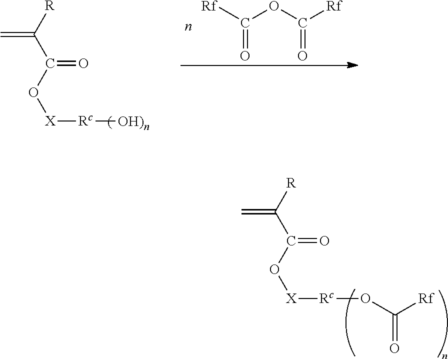

in the above formula, R, X, R^{C}, Rf and n are as defined in connection with the above formula (1).

The compound (i) and a fluorine-containing carboxylic acid are obtained by stirring an aliphatic cyclic hydrocarbon having a hydroxyl group and a (meth)acryloyloxy group via a linking group X with an anhydride of a fluorine-containing carboxylic acid in a solvent such as tetrahydrofuran. After neutralizing and removing the fluorine-containing carboxylic acid by adding sodium bicarbonate or the like to the reaction solution, appropriately carrying out a treatment such as washing by liquid separation, distillation and/or recrystallization enables the compound (i) to be isolated. Alternatively, as a method in which an anhydride of a fluorine-containing carboxylic acid is not used, an esterification reaction of a hydroxyl group in which an acid chloride of a fluorine-containing carboxylic acid may be employed.

### [Structure Unit (II)]

The aforementioned polymer (A) preferably has the structural unit represented by the above formula (2) as the structural unit (II). When the polymer (A) has the structural unit (II) containing fluorine atom(s), the dynamic contact angle of the surface of the resist coating film formed from the radiation-sensitive resin composition can be further improved as a result of enhanced hydrophobicity.

In the above formula (2), R represents a hydrogen atom, a methyl group or a trifluoromethyl group; G represents a single bond, an oxygen atom, a sulfur atom, —CO—O—, —SO₂—O—NH—, —CO—NH— or —O—CO—NH—; and R¹ represents a monovalent chain hydrocarbon group having 1 to 6 carbon atoms and having at least one fluorine atom or a monovalent aliphatic cyclic hydrocarbon group having 4 to 20 carbon atoms and having at least one fluorine atom.

Specific examples of the chain hydrocarbon group having 1 to 6 carbon atoms and having at least one fluorine atom represented by the R¹ described above include a trifluoromethyl group, a 2,2,2-trifluoroethyl group, a perfluoroethyl group, a 2,2,3,3,3-pentafluoropropyl group, a 1,1,1,3,3,3-hexafluoropropyl group, a perfluoro n-propyl group, a perfluoro i-propyl group, a perfluoro n-butyl group, a perfluoro i-butyl group, a perfluoro t-butyl group, a 2,2,3,3,4,4,5,5-octafluoropentyl group, a perfluorohexyl group, and the like.

Specific examples of the aliphatic cyclic hydrocarbon group having 4 to 20 carbon atoms and having at least one n fluorine atom represented by the R¹ described above include a monofluorocyclopentyl group, a difluorocyclopentyl group, a perfluorocyclopentyl group, a monofluorocyclohexyl group, a difluorocyclopentyl group, a perfluorocyclohexylmethyl group, a fluoronorbornyl group, a fluoroadamantyl group, a fluorobornyl group, a fluoroisobornyl group, a fluorotricyclodecyl group, a fluorotetracyclodecyl group, and the like.

Examples of the monomer that gives the structural unit (II) include trifluoromethyl(meth)acrylic acid esters, 2,2,2-trifluoroethyl(meth)acrylic acid esters, perfluoroethyl(meth)acrylic acid esters, perfluoro n-propyl(meth)acrylic acid esters, perfluoro i-propyl(meth)acrylic acid esters, perfluoro n-butyl(meth)acrylic acid esters, perfluoro i-butyl(meth)acrylic acid esters, perfluoro t-butyl(meth)acrylic acid esters, 2-(1,1,1,3,3,3-hexafluoropropyl)(meth)acrylic acid esters, 1-(2,2,3,3,4,4,5,5-octafluoropentyl)(meth)acrylic acid esters, perfluorocyclohexylmethyl(meth)acrylic acid esters, 1-(2,2,3,3,3-pentafluoropropyl)(meth)acrylic acid esters, monofluorocyclopentyl(meth)acrylic acid esters, difluorocyclopentyl(meth)acrylic acid esters, perfluorocyclopentyl(meth)acrylic acid esters, monofluorocyclohexyl(meth)acrylic acid esters, difluorocyclopentyl(meth)acrylic acid esters, perfluorocyclohexylmethyl(meth)acrylic acid esters, fluoronorbornyl(meth)acrylic acid esters, fluoroadamantyl(meth)acrylic acid esters, fluorobornyl(meth)acrylic acid esters, fluoroisobornyl(meth)acrylic acid esters, fluorotricyclodecyl(meth)acrylic acid esters, fluorotetracyclodecyl(meth)acrylic acid esters, and the like.

The content of the structural unit (II) with respect to the entire structural units constituting the polymer (A) is preferably 0 to 50 mol %, more preferably 0 to 30 mol %, and particularly preferably 5 to 20 mol %. When the content falls within this range, a greater dynamic contact angle of the surface of the resist coating film can be provided during the liquid immersion lithography. It is to be noted that the polymer (A) may include the structural unit (II) either alone of one type, or in combination of two or more types thereof.

### [Structure Unit (III)]

The aforementioned polymer (A) preferably has the structural unit represented by the above formula (3) as the structural unit (II). When the polymer (A) has the structural unit (III) containing fluorine atom(s), the dynamic contact angle of the surface of the resist coating film formed from the radiation-sensitive resin composition can be further improved as a result of enhanced hydrophobicity.

In the above formula (3), R represents a hydrogen atom, a methyl group or a trifluoromethyl group; R² represents a hydrocarbon group having 1 to 20 carbon atoms and having a valency of (m+1), and a structure in which an oxygen atom, a sulfur atom, —NR′—, carbonyl group, —CO—O— or —CO—NH— is bonded to the end of R² on the side of R³ is also included; R′ represents a hydrogen atom or a monovalent organic group; R³ represents a single bond, a bivalent chain hydrocarbon group having 1 to 10 carbon atoms or a bivalent aliphatic cyclic hydrocarbon group having 4 to 20 carbon atoms; X² represents a bivalent chain hydrocarbon group having 1 to 20 carbon atoms and having at least one fluorine atom; A represents an oxygen atom, —NR″—, —CO—O—* or —SO₂—O—*; R″ represents a hydrogen atom or a monovalent organic group; * denotes a binding site that binds to R⁴; R⁴ represents a hydrogen atom or a monovalent organic group; and m is an integer of 1 to 3, wherein, provided that m is 2 or 3, the R³, X², A and R⁴ present in plural number may be each the same or different.

The R⁴ described above preferably represents a hydrogen atom, since the solubility of the polymer (A) in an alkaline developer can be enhanced.

The monovalent organic group represented by the R⁴ is exemplified by an acid-dissociable group, an alkali-dissociable group or a hydrocarbon group having 1 to 30 carbon atoms which may have substituent.

The “acid-dissociable group” as referred to means a group that substitutes for a hydrogen atom in a polar functional group such as, for example, a hydroxyl group or a carboxyl group, and is dissociated by an action of an acid. Accordingly, the structural unit (III) consequently yields a polar group by the action of an acid. Therefore, the case in which the R⁴ is an acid-dissociable group is preferred in that the solubility of an exposed area in an alkaline developer can be increased in an exposing process in a method for forming a resist pattern described later.

The “alkali-dissociable group” as referred to means a group that substitutes for a hydrogen atom in a polar functional group such as, for example, a hydroxyl group or a carboxyl group, and is dissociated in the presence of an alkali (in, for example, 2.38% by mass aqueous solution of tetramethylammonium hydroxide at 23° C.). Accordingly, the structural unit (III) consequently yields a polar group by way of an action of an alkali. Therefore, the case in which the R⁴ represents an alkali-dissociable group is preferred since the solubility in an alkaline developer can be improved, and the hydrophobicity of the surface of the resist coating film after the development can be further decreased.

Specific examples of the acid-dissociable group include a t-butoxycarbonyl group, a tetrahydropyranyl group, a tetrahydrofuranyl group, a (thiotetrahydropyranylsulfanyl)methyl group, a (thiotetrahydrofuranylsulfanyl)methyl group, as well as an alkoxy-substituted methyl group, an alkylsulfanyl-substituted methyl group, and the like. It is to be noted that the alkoxy n substituent in the alkoxy-substituted methyl group is exemplified by an alkoxy group having 1 to 4 carbon atoms. In addition, the alkyl group in the alkylsulfanyl-substituted methyl group is exemplified by an alkyl group having 1 to 4 carbon atoms. In addition, the acid-dissociable group may also be group represented by a formula (Y-1) described in a paragraph of a structural unit (IV) described later. Of these, a t-butoxycarbonyl group or an alkoxy-substituted methyl group is preferred in the case in which A in the above formula (3) represents an oxygen atom or —NR″—. Alternatively, in the case in which A in the above formula (3) represents —CO—O—, a group represented by a formula (Y-1) described in a paragraph of a structural unit (IV) described later is preferred.

Specific examples of the alkali-dissociable group include groups represented by the following formulae (W-1) to (W-4). Among these, in the case in which A in the above formula (3) represents an oxygen atom or —NR″—, a group represented by the following formula (W-1) is preferred. Alternatively, in the case in which A in the formula (3) represents —CO—O—, any one group represented by the following formulae (W-2) to (W-4) is preferred.

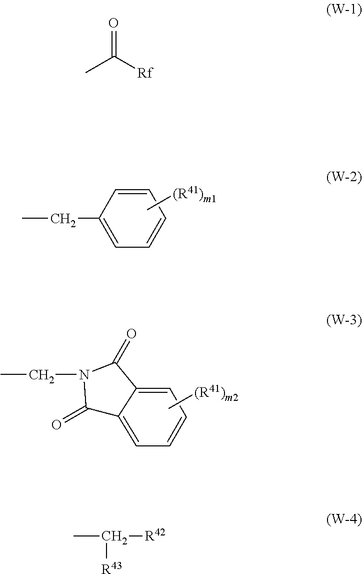

In the above formula (W-1), Rf is as defined in connection with the above formula (1).

In the above formula (W-2) and (W-3), R⁴⁴ represents a substituent, and provided that R⁴⁴ is present in a plural number, the R⁴⁴ present in plural number may be the same or different; m₁ is an integer of 0 to 5; and m₂ is an integer of 0 to 4.

In the above formula (W-4), R⁴² and R⁴³ each independently represent a hydrogen atom or an alkyl group having 1 to 10 carbon atoms, and optionally, R⁴² and R⁴³ bind with each other to form a bivalent aliphatic cyclic hydrocarbon group having 4 to 20 carbon atoms together with the carbon atom to which R⁴² and R⁴³ each bind.

Examples of the substituent represented by the R⁴¹ are identical to the examples of the substituent represented by the R^{s} described above.

Examples of the bivalent aliphatic cyclic hydrocarbon group formed by binding of the R⁴² and R⁴³ with each other together with the carbon atom to which R⁴² and R⁴³ each bind include a cyclopentanediyl group, a methylcyclopentanediyl group, an ethylcyclopentanediyl group, a cyclohexanediyl group, a methylcyclohexanediyl group, an ethylcyclohexanediyl group, a cycloheptanediyl group, a methylcycloheptanediyl group, an ethylcycloheptanediyl group, a 2-norbornanediyl group, a 2-adamantanediyl group, and the like.

Specific examples of the group represented by the above formula (W-4) include a methyl group, an ethyl group, a 1-propyl group, a 2-propyl group, a 1-butyl group, a 2-butyl group, a 1-pentyl group, a 2-pentyl group, a 3-pentyl group, a 1-(2-methylbutyl) group, a 1-(3-methylbutyl) group, a 2-(3-methylbutyl) group, a neopentyl group, a 1-hexyl group, a 2-hexyl group, a 3-hexyl group, a 1-(2-methylpentyl) group, a 1-(3-methylpentyl) group, a 1-(4-methylpentyl) group, a 2-(3-methylpentyl) group, a 2-(4-methylpentyl) group, a 3-(2-methylpentyl) group, and the like. Among these, a methyl group, an ethyl group, a 1-propyl group, a 2-propyl group, a 1-butyl group, and a 2-butyl group are preferred.

Specific examples of the bivalent chain hydrocarbon group having 1 to 20 carbon atoms and having at least one fluorine atom represented by the X² described above include groups represented by the following formulae (X2-1) to (X2-6).

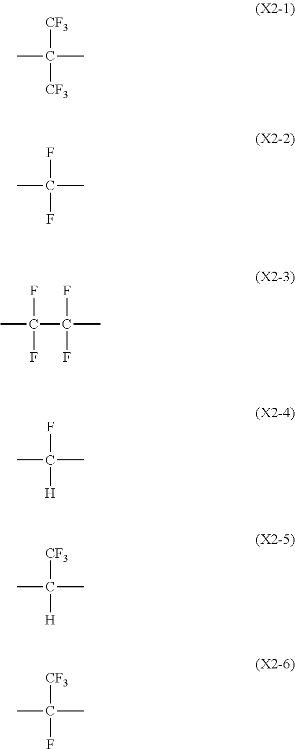

The X² is preferably represented by the above formula (X2-1) in the case in which A in the above formula (3) represents an oxygen atom. Alternatively, in the case in which A in the above formula (3) represents —CO—O—, any one of the groups represented by the above formulae (X2-2) to (X2-6) is preferred, and the group represented by the above formula (X2-2) is more preferred.

It is to be noted that m in the above formula (3) is an integer of 1 to 3. Therefore, R⁴ in the number of 1 to 3 is introduced into the structural unit (III). When m is 2 or 3, R³, R⁴, X² and A are each independently selected. In other words, when m is 2 or 3, the R⁴ present in a plurality of number may have the same structure or the structure different from one another. Also, when m is 2 or 3, the R³ present in a plurality of number may bind to an identical carbon atom, or the distinct carbon atom of R².

Specific examples of the structural unit (III) include the structural units represented by the following formulae (3-1a) to (3-1c).

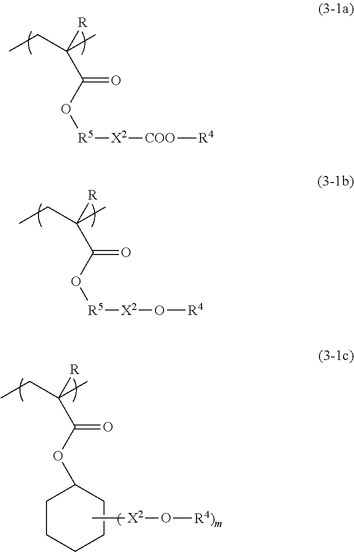

In the above formulae (3-1a) to (3-1c), R⁵ represents a bivalent linear, branched or cyclic saturated or unsaturated hydrocarbon group having 1 to 20 carbon atoms; and X², R⁴ and m are as defined in connection with the above formula (3), and provided that m is 2 or 3, the X² and R⁴ present in plural number may be each the same or different.

Specific examples of the monomer that gives the aforementioned structural unit (III) may include compounds represented by the following formulae (3 m-1) to (3 m-6).

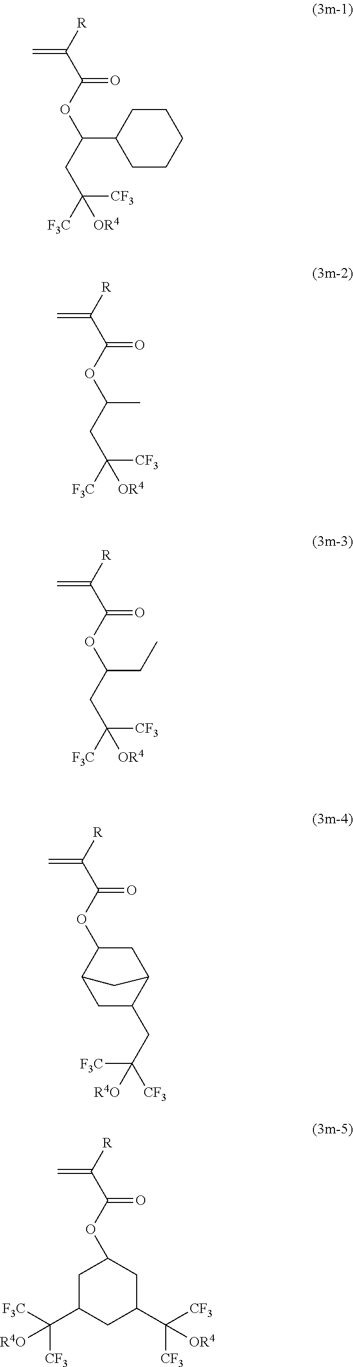

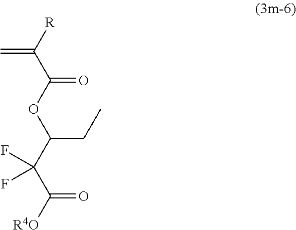

In the formulae (3 m-1) to (3 m-6), R is as defined in connection with the above formula (3); and R⁴ each independently represents a hydrogen atom or a monovalent organic group.

The content of the structural unit (III) is, with respect to the entire structural units constituting the polymer (A), preferably 0 to 90 mol %, more preferably 5 to 85 mol %, and particularly preferably 10 to 80 mol %. When the content falls within such a range, the surface of the resist coating film formed from the radiation-sensitive resin composition can attain an improved extent of decrease of the dynamic contact angle development with an alkali. It is to be noted that the polymer (A) may include the structural unit (III) either alone of one type, or in combination of two or more types thereof.

### [Structure Unit (IV)]

The polymer (A) may have a structural unit (IV) represented by the following formula (4). When the polymer (A) includes the structural unit (IV), the shape of the resist pattern following the development can be further improved.

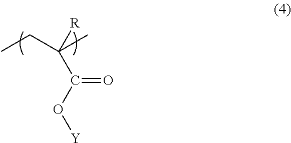

In the above formula (4), R represents a hydrogen atom, a methyl group or a trifluoromethyl group; and Y represents an acid-dissociable group.

The acid-dissociable group represented by the Y described above is preferably a group represented by the following formula (Y-1).

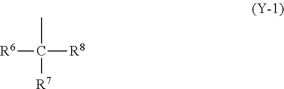

In the above formula (Y-1), R⁶, R⁷ and R⁸ each independently represent an alkyl group having 1 to 4 carbon atoms or having 4 to 20 carbon atoms monovalent aliphatic cyclic hydrocarbon group, and optionally, R⁷ and R⁸ bind with each other to form a bivalent aliphatic cyclic hydrocarbon n group having 4 to 20 carbon atoms together with the carbon atom to which R⁷ and R⁸ are attached.

In the above formula (Y-1), among the groups represented by R⁶, R⁷ and R⁸, examples of the alkyl group having 1 to 4 carbon atoms include a methyl group, an ethyl group, a n-propyl group, an i-propyl group, a n-butyl group, a 2-methylpropyl group, a 1-methylpropyl group, a t-butyl group, and the like. In addition, examples of the monovalent aliphatic cyclic hydrocarbon group having 4 to 20 carbon atoms, or the bivalent aliphatic cyclic hydrocarbon group having 4 to 20 carbon atoms formed by binding of R⁷ and R⁸ with each other together with the carbon atom to which R⁷ and R⁸ are attached include groups having a bridged skeleton such as an adamantane skeleton or a norbornane skeleton, a monocyclic cycloalkane skeleton such as cyclopentane or cyclohexane; and groups having an aliphatic cyclic hydrocarbon skeleton obtained by substituting these groups with one or more linear, branched or cyclic alkyl groups having 1 to 10 carbon atoms such as e.g., a methyl group, an ethyl group, a n-propyl group or an i-propyl group. Among these, groups having a monocyclic cycloalkane skeleton are preferred in view of possibility of further improving a shape of a resist pattern after development.

Specific examples of the structural unit (IV) include structural units represented by the following formulae (4-1) to (4-4).

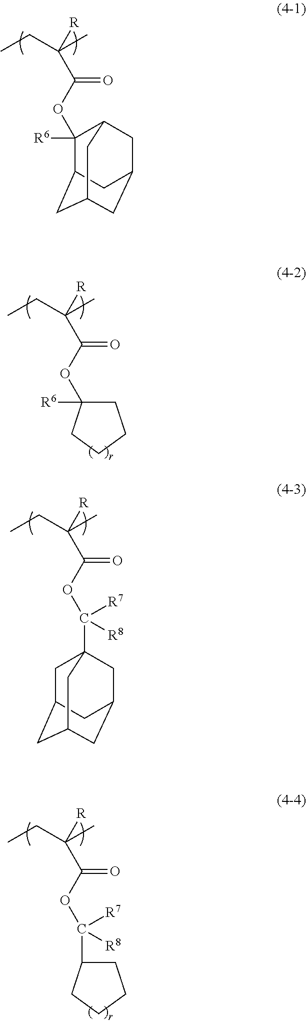

In the above formulae (4-1) to (4-4), R is as defined in connection with the above formula (4); R⁶, R⁷ and R⁸ are as defined in connection with the above formula (Y-1); R⁷ and R⁸ may bind with each other to form a bivalent aliphatic cyclic hydrocarbon group having 4 to 20 carbon atoms together with the carbon atom to which R⁷ and R⁸ are attached; and r is each independently an integer of 1 to 3.

The content of the structural unit (IV) is with respect to the entire structural units constituting the polymer (A), preferably no greater than 70 mol %, more preferably 5 to 60 mol %, and particularly preferably 5 to 50 mol %. When the content falls within this range, the resist pattern configuration after development can be further improved. In addition, the polymer (A) may have the structural unit (IV) either alone of one type, or in combination of two or more types thereof.

### [Structural Unit (V)]

The polymer (A) may have a structural unit having an alkali-soluble group (hereinafter, may be also referred to as “structural unit (V)”). When the polymer (A) includes the structural unit (V), the affinity to the developer can be improved.

The alkali-soluble group in the aforementioned structural unit (V) is preferably a functional group having hydrogen atom(s) and a pKa of 4 to 11 in light of improvement of the solubility in the developer. Such a functional group is exemplified by a functional group represented by the following formulae (5s-1) and (5s-2), and the like.

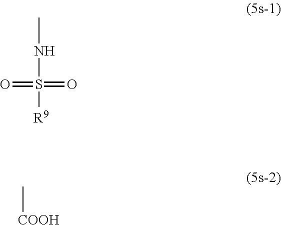

In the above formula (5s-1), R⁹ represents a hydrocarbon group having 1 to 10 carbon atoms and having at least one fluorine atom.

The hydrocarbon group having 1 to 10 carbon atoms and having at least one fluorine atom represented by the R⁹ described above is not particularly limited as long as a part or all of hydrogen atoms of the hydrocarbon group having 1 to 10 carbon atoms are substituted by a fluorine atom, and in particular, a trifluoromethyl group is preferred.

The monomer that gives the structural unit (V) is not particularly limited, and a methacrylic acid ester, an acrylic acid ester or an α-trifluoro ester acrylate is preferred.

Specific examples of the structural unit (V) include structural units represented by the following formulae (5-1) and (5-2).

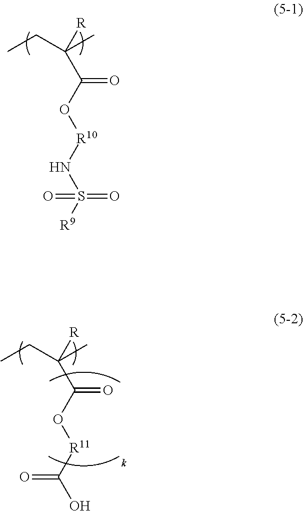

In the above formulae (5-1) and (5-2), R represents a hydrogen atom, a methyl group or a trifluoromethyl group; R⁹ is as defined in connection with the above formula (5s-1); and R¹⁰ represents a single bond or a bivalent linear, branched or cyclic, saturated or unsaturated hydrocarbon group having 1 to 20 carbon atoms. In the above formula (5-2), R¹¹ represents a bivalent linking group; and k is 0 or 1.

Examples of the bivalent linking group represented by the R¹¹ described above include those identical to examples of the bivalent linking group X in the structural unit (I).

Specific examples of the structural unit (V) include structural units represented by the following formulae (5-1a), (5-1b) and (5-2a) to (5-2e).

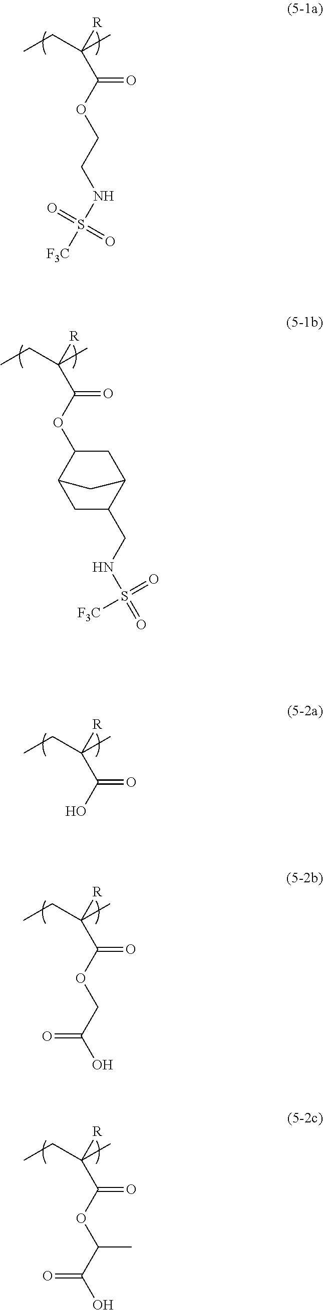

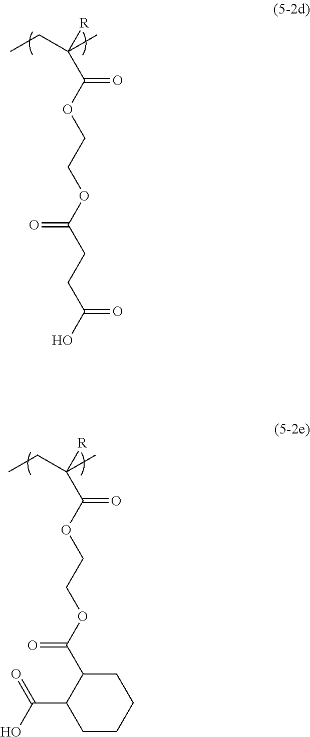

In the above formulae (5-1a), (5-1b) and (5-2a) to (5-2e), R each independently represents a hydrogen atom, a methyl group or a trifluoromethyl group.

The content of the structural unit (V) is, with respect to the entire structural units constituting the polymer (A), typically no greater than 50 mol %, preferably 5 to 30 mol %, and more preferably 5 to 20 mol %. When the content falls within this range, securement of the scan following capability and water repellency during liquid immersion lithography, and the affinity to the developer during development can be achieved with a good balance.

### [Structural Unit (VI)]

The aforementioned polymer (A) may have a structural unit (VI) represented by the following formula (6). When the polymer (A) includes the structural unit (VI), the affinity to the developer can be improved.

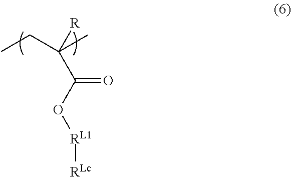

In the above formula (6), R represents a hydrogen atom, a methyl group or a trifluoromethyl group; R^{L1} represents a single bond or a bivalent linking group; and R^{Lc} represents a monovalent organic group having a lactone structure or a monovalent organic group having a cyclic carbonate structure.

Examples of the bivalent linking group represented by the R^{L1} include those identical to examples of the bivalent linking group X in the structural unit (I).

The monovalent organic group having a lactone structure represented by the R^{Lc} described above is exemplified by groups represented by the following formulae (Lc-1) to (Lc-6), and the like.

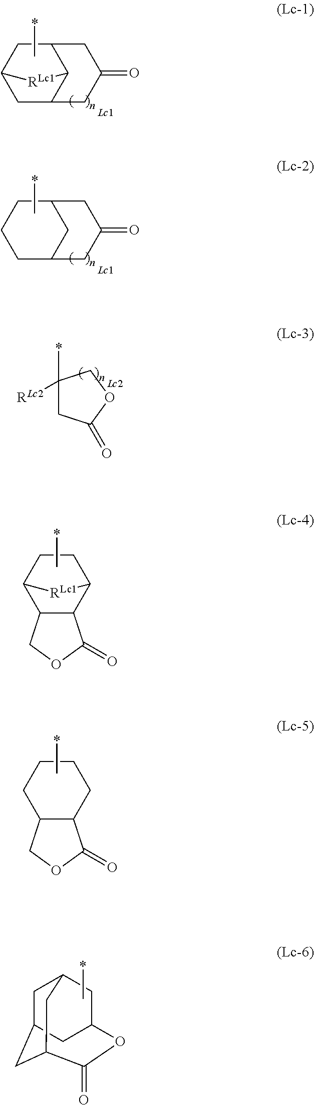

In the above formulae (Lc-1) to (Lc-6), R^{Lc1} each independently represents an oxygen atom or a methylene group; R^{Lc2} represents a hydrogen atom or an alkyl group having 1 to 4 carbon atoms; n_{Lc1} is each independently 0 or 1; n_{Lc2} is an integer of 0 to 3; * denotes a binding site that binds to R^{L1} in the above formula (6); and the group represented by the above formulae (Lc-1) to (Lc-6) may have a substituent.

Examples of the substituent included in the group represented by the above formulae (Lc-1) to (Lc-6) include those identical to examples of the substituent included in R^{C} in the structural unit (I).

Specific examples of the structural unit (VI) include those disclosed in paragraphs nos. [0054] to [0057] of Japanese Unexamined Patent Application, Publication No. 2007-304537, structural units disclosed in paragraphs nos. [0086] to [0088] of Japanese Unexamined Patent Application, Publication No. 2008-088343, structural units represented by the following formulae (6-1a) to (6-11), and the like.

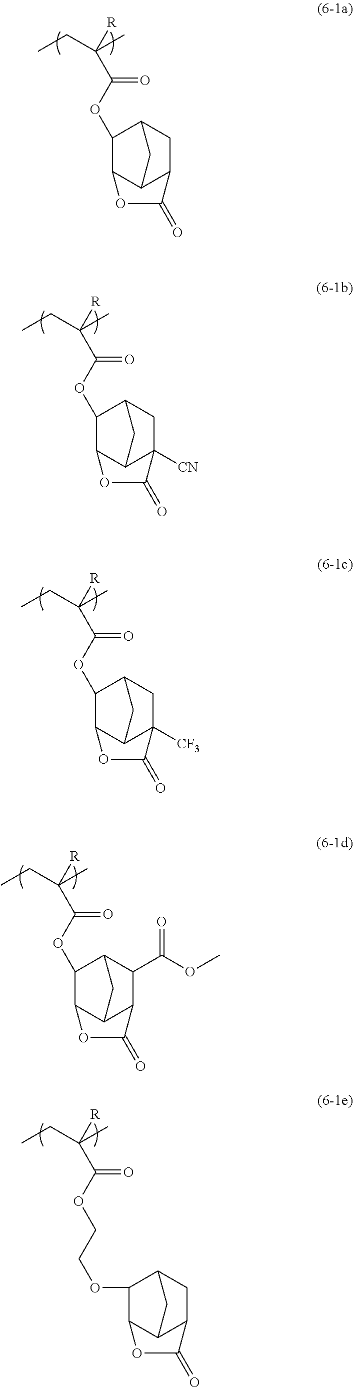

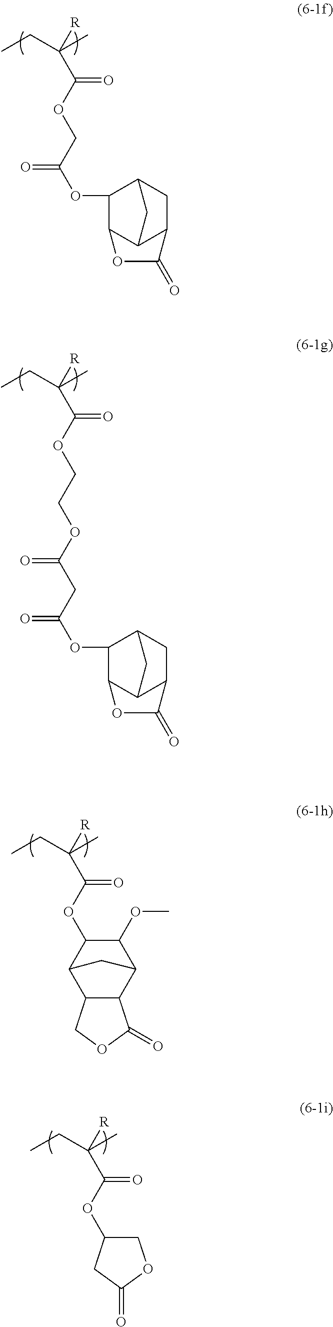

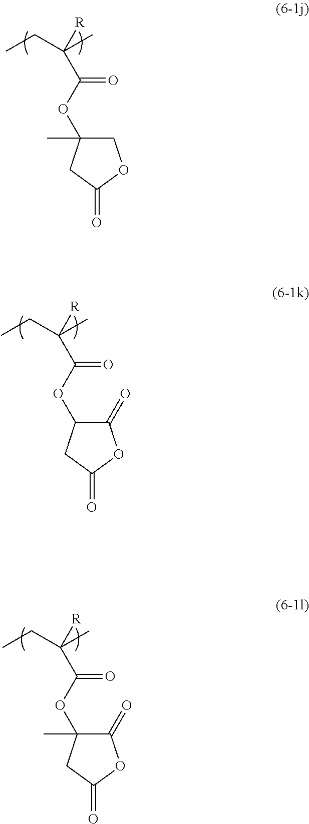

In the above formulae (6-1a) to (6-11), R represents a hydrogen atom, a methyl group or a trifluoromethyl group.

It is to be noted that the aforementioned structural unit (VI) may be included either alone of one type or in combination of two or more types thereof. A preferable monomer that gives the structural unit (VI) is exemplified by monomers described in paragraph [0043] of PCT International Publication No. 2007/116664.

Among the candidates of the structural unit (VI), the structural unit having a cyclic carbonate structure is exemplified by the structural unit represented by the following formula (6-2a), and the like.

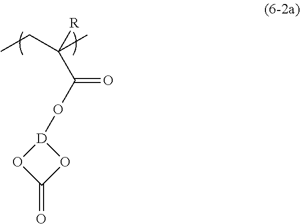

In the above formula (6-2a), R is as defined in connection with the above formula (6); D represents a trivalent chain hydrocarbon group having 1 to 30 carbon atoms, a trivalent aliphatic cyclic hydrocarbon group having 3 to 30 carbon atoms or a trivalent aromatic hydrocarbon group having 6 to 30 carbon atoms; D may have an oxygen atom, a carbonyl group, or —NH— in its skeleton; or alternatively D may have a substituent.

Examples of the substituent which may be included in the D include those identical to examples of the substituent of R^{C} in the structural unit (I) described above.

The monomer that gives the structural unit represented by the above formula (6-2a) may be synthesized by conventionally well-known methods described in, for example, Tetrahedron Letters, Vol. 27, No. 32 p. 3741 (1986); and Organic Letters, Vol. 4, No. 15 p. 2561 (2002).

Preferable examples of the structural unit represented by the above formula (6-2a) include structural units represented by the following formulae (6-2a-1) to (6-2a-22).

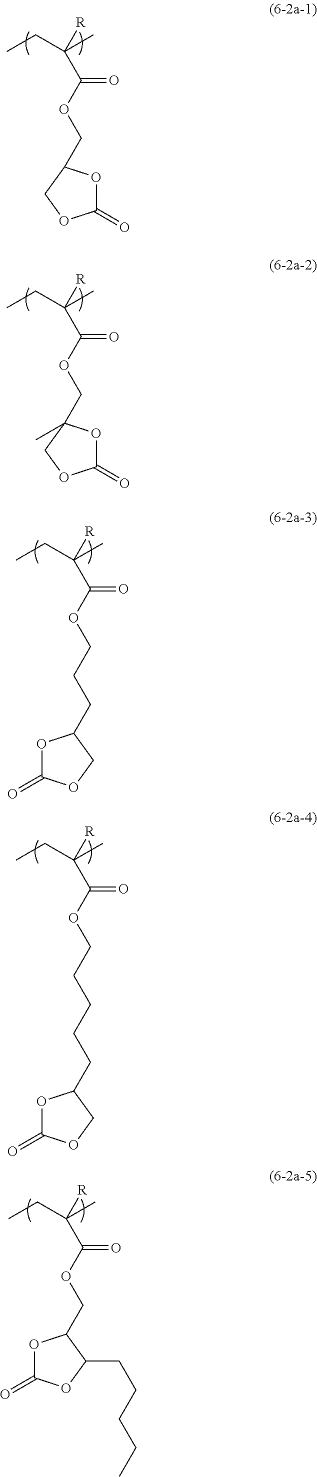

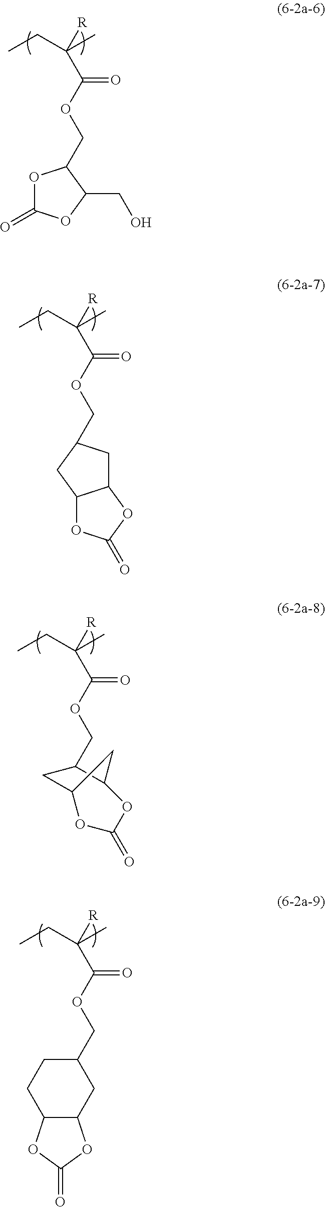

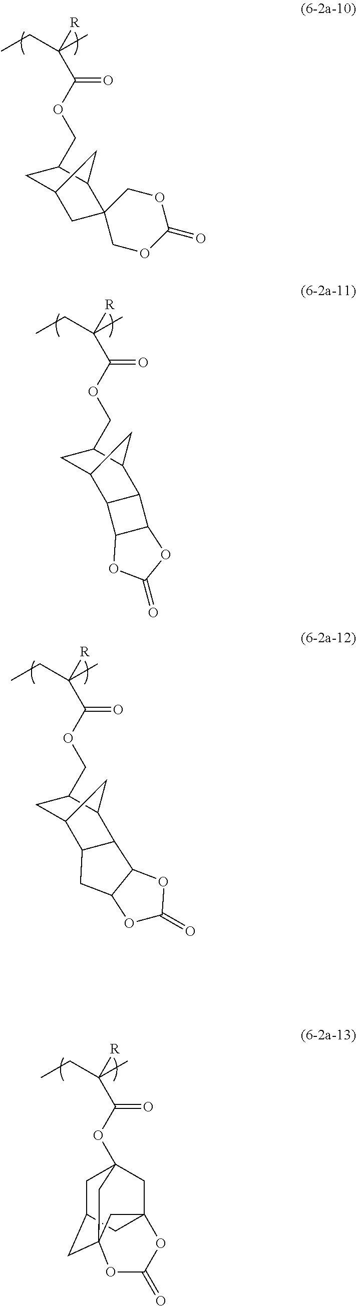

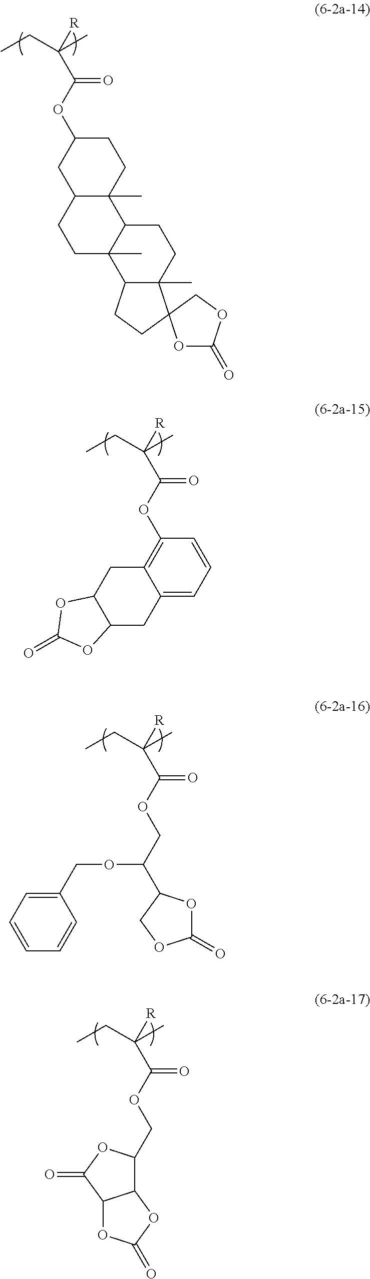

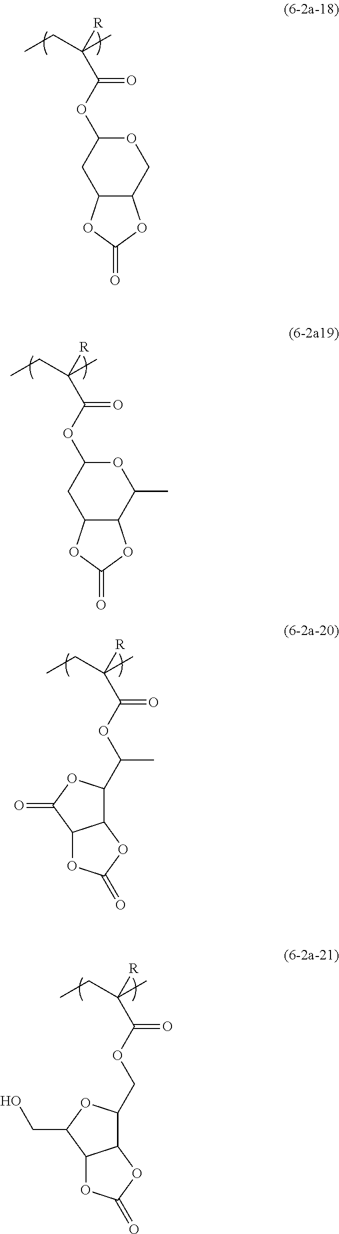

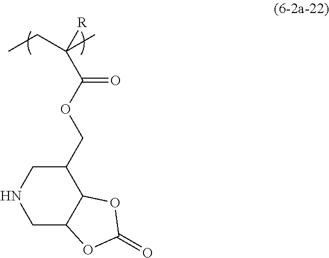

The content of the structural unit (VI) with respect to the entire structural units constituting the polymer (A) is typically no greater than 50 mol %, preferably no greater than 40 mol %, and more preferably no greater than 5 to 30 mol %. When the content falls within such a range, a great dynamic contact angle during the liquid immersion lithography, as well as enough decrease of the dynamic contact angle by way of the development can be achieved.

### [Structural Unit (VII)]

The polymer (A) may have a structural unit (VII) represented by the following formula (7). When the polymer (A) includes the structural unit (VII), the affinity to the developer can be improved.

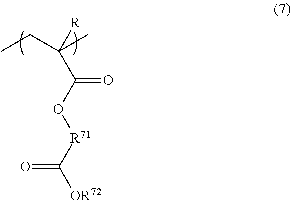

In the above formula (7), R represents a hydrogen atom, a methyl group or a trifluoromethyl group; R⁷¹ represents a bivalent linking group not having a fluorine atom; and R⁷² represents an alkali-dissociable group.

Specific examples of the bivalent linking group not having a fluorine atom represented by the R⁷¹ described above include those identical to examples of the group not having a fluorine atom among the candidates of the bivalent linking group X in the structural unit (I).

Examples of the alkali-dissociable group represented by the R⁷² described above include groups represented by the above formulae (W-2) to (W-4).

Specific examples of the structural unit (VII) include structural units represented by the following formulae.

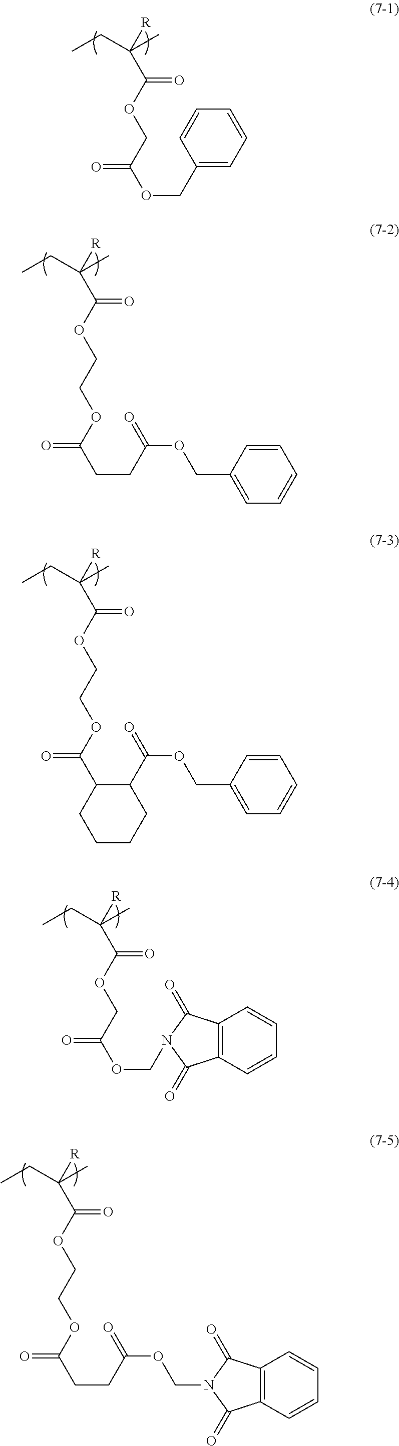

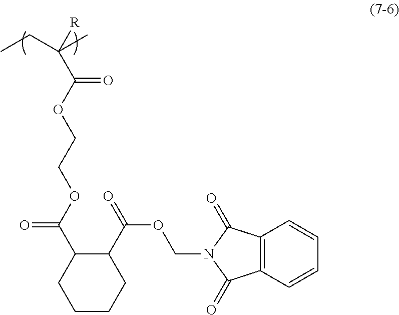

In the above formulae (7-1) to (7-6), R represents a hydrogen atom, a methyl group or a trifluoromethyl group.

The content of the structural unit (VII) with respect to the entire structural units constituting the polymer (A) is typically no greater than 50 mol %, preferably no greater than 40 mol %, and more preferably 5 to 20 mol %. When the content falls within such a range, a great dynamic contact angle during liquid immersion lithography, as well as enough decrease of the dynamic contact angle by way of the development can be achieved.

The content of the polymer (A) is, with respect to the entire polymers, i.e., the total of the polymer (A) and other polymer which may be contained as needed in the radiation-sensitive resin composition, 0.1 to 20% by mass is preferred, and more preferably 0.3 to 15% by mass, still more preferably 0.3 to 10% by mass, particularly preferably 0.5 to 10% by mass, and further particularly preferably 1 to 10% by mass. When the content of the polymer (A) is less than 0.1% by mass, site-dependent variation of the dynamic contact angle of the resist coating film obtained from the composition may be caused. To the contrary, when the content exceeds 20% by mass, the difference of dissolution of the resist coating film between the light-exposed site and the site unexposed with light becomes so small that the pattern configuration may be deteriorated.

### <Method for Synthesizing the Polymer (A)>

The aforementioned polymer (A) may be synthesized according to a common procedure such as radical polymerization. The polymer (A) is preferably synthesized according to a method such as, e.g.:

(1) a method in which a solution containing a monomer and a radical initiator is added dropwise to a solution containing a reaction solvent or a monomer to permit a polymerization reaction;

(2) a method in which a solution containing a monomer, and a solution containing a radical initiator are each separately added dropwise to a solution containing a reaction solvent or a monomer to permit a polymerization reaction;

(3) a method in which a plurality of solutions each containing a monomer, and a solution containing a radical initiator are each separately added dropwise to a solution containing a reaction solvent or a monomer to permit a polymerization reaction; or

(4) a method in which a solution containing a monomer and a radical initiator is subjected to a polymerization reaction in the absence of a solvent or in a reaction solvent.

It is to be noted that when the reaction is allowed by adding a monomer solution dropwise to a monomer solution, the amount of the monomer in the monomer solution added is preferably no less than 30 mol %, more preferably no less than 50 mol %, and particularly preferably no less than 70 mol % with respect to the total amount of the monomers used in the polymerization.

The reaction temperature in these methods may be determined ad libitum depending of the type of the initiator species. The reaction temperature is usually 30 to 150° C., preferably 40 to 150° C., and more preferably 50° C. to 140° C. The time period for the dropwise addition may vary depending on the conditions such as the reaction temperature, the type of the initiator and the monomer to be reacted, but is usually 30 min to 8 hrs, preferably 45 min to 6 hrs, and more preferably 1 to 5 hrs. Further, the total reaction time period including the time period for dropwise addition may also vary depending on the conditions similarly to the time period for the dropwise addition, and is typically 30 min to 12 hrs, preferably 45 min to 12 hrs, and more preferably 1 to 10 hrs.

The radical initiator for use in the polymerization is exemplified by azo radical initiators such as azobisisobutyronitrile (AIBN), 2,2′-azobis(4-methoxy-2,4-dimethylvaleronitrile), 2,2′-azobis(2-n cyclopropylpropionitrile), 2,2′-azobis(2,4-dimethylvaleronitrile) and dimethyl 2,2′-azobis(2-methylpropionate); peroxide radical initiators such as benzoylperoxide, t-butylhydroperoxide and cumenehydroperoxide, and the like. Of these, AIBN and dimethyl 2,2′-azobis(2-methylpropionate) are preferred. These radical initiators may be used either alone, or as a mixture of two or more thereof.

As the solvent for polymerization, any solvent other than solvents that inhibit the polymerization (nitrobenzene having a polymerization inhibitory effect, mercapto compounds having a chain transfer effect, etc.), and which is capable of dissolving the monomer may be used. For example, alcohols, ethers, ketones, amides, ester-lactones, nitriles and mixed solvents thereof, and the like may be included. These solvents may be used either alone, or as a mixture of two or more thereof.

The polymer obtained by the polymerization reaction may be recovered preferably by a reprecipitation technique. More specifically, after the polymerization reaction is completed, the polymerization mixture is charged into a solvent for reprecipitation, whereby a target polymer is recovered in the form of powder. As the reprecipitation solvent, an alcohol, an alkane or the like may be used either alone or as a mixture of two or more thereof. Further, alternatively to the reprecipitation technique, liquid separating operation, column operation, ultrafiltration operation or the like may be employed to recover the polymer through eliminating low molecular components such as monomers and oligomers.

The polystyrene equivalent weight average molecular weight (hereinafter, may be also referred to as “Mw”) of the polymer (A) as determined by gel permeation chromatography (GPC) is not particularly limited, and preferably 1,000 to 50,000, more preferably 1,000 to 40,000, and particularly preferably 1,000 to 30,000. The Mw of the polymer (A) being less than 1,000 may lead to failure in obtaining a resist coating film having a satisfactory dynamic contact angle. To the contrary, when the Mw of the polymer (A) exceeds 50,000, developability of the resist coating film may be inferior.

Also, the ratio (Mw/Mn) of Mw to the polystyrene equivalent number average molecular weight (hereinafter, may be also referred to as “Mn”) as determined by GPC of the polymer (A) is typically 1.0 to 5.0, preferably 1.0 to 4.0, and more preferably 1.0 to 2.0.

### <(B) Acid Generator>

The acid generating agent (B) that constitutes the radiation-sensitive resin composition is exemplified by onium salt compounds such as sulfonium salts, tetrahydrothiophenium salts and iodonium salts, organic halogen compounds, sulfone compounds such as disulfones and diazomethanesulfones, sulfonic acid compounds, and the like. The form of the acid generator (B) contained in the radiation-sensitive resin composition may be in the form of either an acid generating agent that is a compound as described later (hereinafter, may be also referred to as appropriately “(B) acid generating agent”) or a form of an acid generating group incorporated as a part of the polymer (A) and/or other polymer such as the polymer (C) described later, or may be in both of these forms.

Suitable specific examples of such an acid generating agent (B) include compounds described in paragraphs nos. [0080 ] to [0113] of Japanese Unexamined Patent Application, Publication No. 2009-134088, and the like.

Specifically, examples of the acid generating agent (B) preferred include:

iodonium salts such as diphenyliodonium trifluoromethanesulfonate, diphenyliodonium nonafluoro-n-butanesulfonate, diphenyliodonium perfluoro-n-octanesulfonate, bis(4-t-butylphenyl)iodonium trifluoromethanesulfonate, bis(4-t-butylphenyl)iodonium nonafluoro-n-butanesulfonate, and bis(4-t-butylphenyl)iodonium perfluoro-n-octanesulfonate;

sulfonium salts such as triphenylsulfonium trifluoromethanesulfonate, triphenylsulfonium nonafluoro-n-butanesulfonate, triphenylsulfonium perfluoro-n-octanesulfonate, cyclohexyl 2-oxocyclohexylmethylsulfonium trifluoromethanesulfonate, dicyclohexyl 2-oxocyclohexylsulfonium trifluoromethanesulfonate, 2-oxocyclohexyldimethylsulfonium trifluoromethanesulfonate, and 4-hydroxy-1-naphthyldimethylsulfonium trifluoromethanesulfonate;

tetrahydrothiophenium salts such as 4-hydroxy-1-naphthyltetrahydrothiophenium trifluoromethanesulfonate, 4-hydroxy-1-naphthyltetrahydrothiophenium nonafluoro-n-butanesulfonate, 4-hydroxy-1-naphthyltetrahydrothiopheniumperfluoro-n-octanesulfonate, 1-(1-naphthylacetomethyl) tetrahydrothiophenium trifluoromethanesulfonate, 1-(1-naphthylacetomethyl)tetrahydrothiophenium nonafluoro-n-butanesulfonate, 1-(1-naphthylacetomethyl)tetrahydrothiopheniumperfluoro-n-octanesulfonate, 1-(3,5-dimethyl-4-hydroxyphenyl)tetrahydrothiophenium trifluoromethanesulfonate, 1-(3,5-dimethyl-4-hydroxyphenyl)tetrahydrothiophenium nonafluoro-n-butanesulfonate, and 1-(3,5-dimethyl-4-hydroxyphenyl)tetrahydrothiopheniumperfluoro-n-octanesulfonate;

sulfonic acid compounds such as trifluoromethanesulfonyl bicyclo[2.2.1]hept-5-ene-2,3-dicarbodiimide, nonafluoro-n-butanesulfonylbicyclo[2.2.1]hept-5-ene-2,3-dicarbodiimide, perfluoro-n-octanesulfonylbicyclo[2.2.1]hept-5-ene-2,3-dicarbodiimide, N-hydroxysuccimidetrifluoromethanesulfonate, N-hydroxysuccimidenonafluoro-n-butanesulfonate, N-hydroxysuccimideperfluoro-n-octanesulfonate, and 1,8-naphthalenedicarboxylic acid imidetrifluoromethanesulfonate.

The acid generating agent (B) may be used either alone or as a mixture of two or more thereof. The content of the acid generating agent (B) with respect to 100 parts by mass of the total amount of the polymers included in the radiation-sensitive resin composition is, in light of securement of the sensitivity and developability as a resist, preferably 0.1 to 30 parts by mass, and more preferably 0.1 to 20 parts by mass. In this case, when the content of the acid generating agent (B) is less than 0.1 parts by mass, the sensitivity and the developability tend to be inferior, whereas when the content exceeds 30 parts by mass, transparency for radioactive rays is lowered, and thus it may be difficult to obtain a rectangular resist pattern.

The radiation-sensitive resin composition preferably contains a polymer having an acid-dissociable group in addition to the polymer (A). Such a polymer having an acid-dissociable group is insoluble or hardly soluble in alkali before being subjected to an action of an acid, and becomes soluble in alkali upon dissociation of the acid-dissociable group by an action of an acid generated from the acid generating agent (B), etc. The phrase “insoluble or hardly soluble in alkali” as referred to for polymers means a property that in a case in which a coating film having a film thickness of 100 nm produced using only such a polymer is developed in place of the resist coating film under conditions of development with an alkali which are employed when resist patterns are formed from the resist coating film that had been n formed with the radiation-sensitive resin composition, no less than 50% of the initial film thickness of the coating film remains after the development.

### <(C) Polymer>

In the radiation-sensitive resin composition of the embodiment of the present invention, the polymer having an acid-dissociable group is preferably a polymer having the content of fluorine atoms than that of the aforementioned polymer (A). When the content of fluorine atoms in the polymer (C) is smaller than the content of fluorine atoms in the polymer (A), tendency of uneven distribution of the polymer (A) in the superficial layer is further enhanced in the resist coating film formed with the radiation-sensitive resin composition containing the polymer (C) and the polymer (A), and thus the hydrophobicity of the polymer (A) and characteristic features in connection with the dynamic contact angle resulting from a decrease of the hydrophobicity can be more effectively achieved. It is to be noted that the content of fluorine atoms (% by mass) can be determined by deciding each structure of the polymer (C) and the polymer (A) by ¹³C-NMR, and calculating based on their structures.

Specific structure of the polymer (C) is not particularly limited as long as it has the properties as described above, and the polymer (C) preferably has the structural unit (III) represented by the above formula (3) and the structural unit (VI) represented by the above formula (6) in regard to the polymer (A).

### [Structural Unit (III)]

The content of the structural unit (III) with respect to the entire structural units constituting the polymer (C) is preferably 0 to 30 mol %, and more preferably 0 to 15 mol %. When the content exceeds 30 mol %, adhesiveness to the substrate may be insufficient, whereby the pattern may be detached.

### [Structural Unit (VI)]

The content of the structural unit (VI) with respect to the entire structural units constituting the polymer (C) is preferably 5 to 75 mol %, more preferably 15 to 65 mol %, and particularly preferably 25 to 55 mol %. When the content is less than 5 mol %, adhesiveness to the substrate as a resist may be insufficient, whereby the pattern may be detached. To the contrary, when the content exceeds 75 mol %, the contrast after dissolution may be impaired, whereby the pattern configuration may be deteriorated.

### [Other Structural Unit]

The polymer (C) may have other structural unit except for the structural unit (III) and the structural unit (VI) as long as it has the content of fluorine atoms described above. A polymerizable unsaturated monomer that gives the other structural unit is exemplified by a monomer disclosed in paragraphs nos. [0065] to [0085] of PCT International n Publication No. 2007/116664A.

The other structural unit is preferably a structural unit derived from 2-hydroxyethyl (meth)acrylate, 2-hydroxypropyl (meth)acrylate, or 3-hydroxypropyl (meth)acrylate; the structural unit (V); and a structural unit represented by the following formula (o-1).

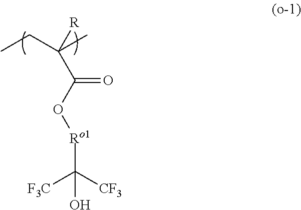

In the above formula (0-1), R represents a hydrogen atom, a methyl group, or trifluoromethyl group; and R^{o1} represents a bivalent linking group.

Examples of the bivalent linking group represented by the R^{o1} described above include those identical to examples of the bivalent linking group X in the above structural unit (I).

The structural unit represented by the above formula (o-1) is exemplified by structural units represented by the following formulae (o-1a) to (o-1 h), and the like.

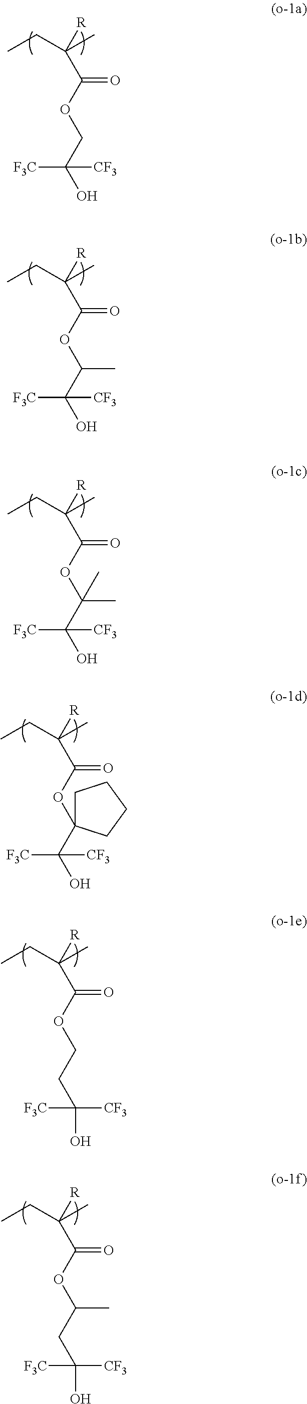

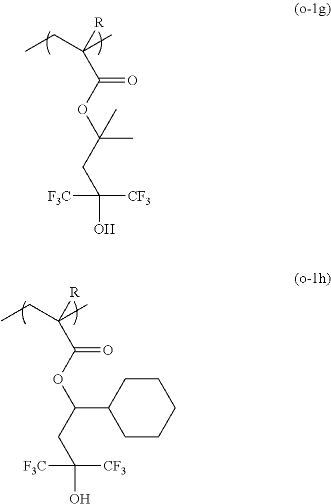

In the above formulae (o-1a) to (o-1 h), R each independently represents a hydrogen atom, a methyl group or a trifluoromethyl group.

The content of the other structural unit with respect to the entire structural units constituting the polymer (C) is typically no greater than 20 mol %, and preferably no greater than 15 mol %. When the content exceeds 20 mol %, pattern formability may be deteriorated.

The Mw of the polymer (C) is typically 3,000 to 300,000, preferably 4,000 to 200,000, and more preferably 4,000 to 100,000. When the Mw is less than 3,000, the heat resistance as a resist may be deteriorated. To the contrary, when the Mw exceeds 300,000, the developability as a resist may be deteriorated.

The content of the polymer (C) in the radiation-sensitive resin composition with respect to the total solid content is typically no less than 70% by mass, and preferably no less than 80% by mass. When the content is less than 70% by mass, resolving performance as a resist may be deteriorated.

### <(D) Acid Diffusion Controller>

The radiation-sensitive resin composition of the embodiment of the present invention may contain an acid diffusion controller if necessary as (D) a component. The acid diffusion controller (D) is exemplified by a compound represented by the following formula (8) (hereinafter, may be also referred to as “nitrogen-containing compound (I)”), a compound having two nitrogen atoms in the same molecule (hereinafter, may be also referred to as “nitrogen-containing compound (II)”), a compound having three or more nitrogen atoms (hereinafter, may be also referred to as “nitrogen-containing compound (III)”), an amide group-containing compound, a urea compound, a nitrogen-containing heterocyclic compound, and the like. When the acid diffusion controller (D) is contained, pattern configuration and dimension fidelity as a resist can be improved. The form of the acid diffusion controller (D) contained in the radiation-sensitive resin composition may be in the form of either an acid diffusion control agent that is a compound as described later (hereinafter, may be also referred to as appropriately “(D) acid diffusion control agent”) or a form of an acid diffusion control group incorporated as a part of the polymer (A) and/or other polymer such as the polymer (C), or may be in both of these forms.

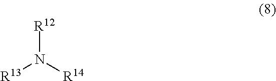

In the above formula (8), R¹² to R¹⁴ each independently represent a hydrogen atom, a substituted or unsubstituted linear, branched or cyclic alkyl group, an aryl group or an aralkyl group.

Examples of the nitrogen-containing compound (I) include monoalkylamines such as n-hexylamine; dialkylamines such as di-n-butylamine; trialkylamines such as triethylamine; aromatic amines such as aniline, and the like.

Examples of the nitrogen-containing compound (II) include ethylenediamine, N,N,N′,N′-tetramethylethylenediamine, and the like.

Examples of the nitrogen-containing compound (III) include polyamine compounds such as polyethyleneimine and polyallylamine; polymers such as dimethylaminoethylacrylamide, and the like.

Examples of the amide group-containing compound include formamide, N-methylformamide, N,N-dimethyl formamide, acetamide, N-methylacetamide, N,N-dimethylacetamide, propionamide, benzamide, pyrrolidone, N-methylpyrrolidone, and the like.

Examples of the urea compound include urea, methylurea, 1,1-dimethylurea, 1,3-dimethylurea, 1,1,3,3-tetramethylurea, 1,3-diphenylurea, tributylthiourea, and the like.

Examples of the nitrogen-containing heterocyclic compound include pyridines such as pyridine and 2-methylpyridine, pyrazine, pyrazole, and the like.

In addition, as the aforementioned nitrogen-containing organic compound, a compound having an acid-dissociable group may be also used. Examples of the nitrogen-containing organic compound having such an acid-dissociable group include N-(t-butoxycarbonyl)piperidine, N-(t-butoxycarbonyl)imidazole, N-(t-butoxycarbonyl)benzimidazole, N-(t-butoxycarbonyl)-2-phenylbenzimidazole, N-(t-butoxycarbonyl)di-n-octylamine, N-(t-butoxycarbonyl)diethanolamine, N-(t-butoxycarbonyl)dicyclohexylamine, N-(t-butoxycarbonyl)diphenylamine, N-(t-butoxycarbonyl)-4-hydroxypiperidine, and the like.

Alternatively, as the acid diffusion controller, a compound represented by the following formula (9) may be also used.

X⁺Z⁻ (9)

In the above formula (9), X⁺ is a cation represented by the following formula (9-1-1) or (9-1-2); Z⁻ is OH⁻, an anion represented by R^{D1}—COO⁻, an anion represented by R^{D1}—SO₃, or an anion represented by R^{D1}—N⁻—SO₂—R^{D2}, R^{D1} represents an alkyl group which is unsubstituted or substituted, a monovalent aliphatic cyclic hydrocarbon group or an aryl group; R^{D2} represents a monovalent aliphatic cyclic hydrocarbon group, or an alkyl group in which a part or all hydrogen atoms are substituted by a fluorine atom.

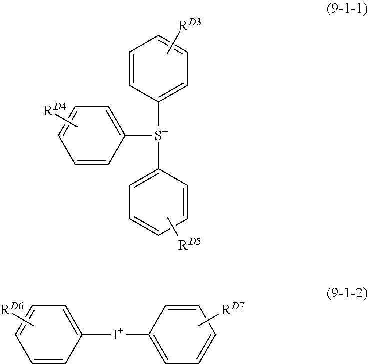

In the above formula (9-1-1), R^{D3} to R^{D5} each independently represent a hydrogen atom, an alkyl group, an alkoxy group, a hydroxyl group or a halogen atom; and in the above formula (9-1-2), R^{D6} and R^{D7} each independently represent a hydrogen atom, an alkyl group, an alkoxy group, a hydroxyl group or a halogen atom.

The aforementioned compound is used as an acid diffusion controller (hereinafter, may be also referred to as “photodegradable acid diffusion controller”) that loses acid diffusion controllability upon decomposition by exposure. When this compound is contained, the acid is diffused at a site exposed with light, whereas diffusion of the acid is controlled at a site unexposed with light, whereby an excellent contrast between the site exposed with light and the site unexposed with light is attained (i.e., a boundary between the light-exposed site and the site unexposed with light becomes clear). Therefore, it is particularly effective in improving the resolving performance as a resist of the radiation-sensitive resin composition of the embodiment of the present invention.

X⁺ in the above formula (9) is a cation represented by the above formula (9-1-1) or (9-1-2). Furthermore, R^{D3} to R^{D5} in the above formula (9-1-1) each independently represent a hydrogen atom, an alkyl group, an alkoxyl group, a hydroxyl group or a halogen atom, and among these, a hydrogen atom, an alkyl group, an alkoxy group, and a halogen atom are preferred due to having an effect of decreasing the solubility of the compound in the developer. Moreover, R^{D6} and R^{D7} in the above formula (9-1-2) each independently represent a hydrogen atom, an alkyl group, an alkoxyl group, a hydroxyl group, or halogen atom, and of these, a hydrogen atom, an alkyl group, or a halogen atom is preferred.

Z⁻ in the above formula (9) is OH⁻, an anion represented by R^{D1}—COO⁻, an anion represented by R^{D1}—SO₃⁻ or an anion represented by the formula of R^{D1}—N⁻—SO₂—R^{D2}, wherein, R^{D1} in these formulae represents an alkyl group which is unsubstituted or substituted, an aliphatic cyclic hydrocarbon group or an aryl group, and of these, an aliphatic cyclic hydrocarbon group or an aryl group is preferred due to having an effect of decreasing the solubility of the compound in the developer.

Examples of the alkyl group which is unsubstituted or substituted in the above formula (9) include hydroxyalkyl groups having 1 to 4 carbon atoms such as a hydroxymethyl group; alkoxyl groups having 1 to 4 carbon atoms such as a methoxy group; a cyano group; groups having one or more substituents such as a cyano alkyl group having 2 to 5 carbon atoms such as a cyano methyl group, and the like. Of these, a n hydroxymethyl group, a cyano group, and a cyano methyl group are preferred.

Examples of the unsubstituted or substituted aliphatic cyclic hydrocarbon group in the above formula (9) include monovalent groups derived from an aliphatic cyclic hydrocarbon having e.g.: a monocyclic cycloalkane skeleton such as hydroxycyclopentane, hydroxycyclohexane or cyclohexanone; a bridged aliphatic cyclic hydrocarbon skeleton such as 1,7,7-trimethyl bicyclo[2.2.1]heptan-2-one (camphor), and the like. Of these, groups derived from 1,7,7-trimethyl bicyclo[2.2.1]heptan-2-one are preferred.

Examples of the aryl group which is unsubstituted or substituted in the above formula (9) include a phenyl group, a benzyl group, a phenylethyl group, a phenylpropyl group, a phenylcyclohexyl group and the like, and groups obtained by substituting a part or all of hydrogen atoms of these groups by a hydroxyl group, a cyano group or the like, and the like. Of these, a phenyl group, a benzyl group or a phenylcyclohexyl group is preferred.

Z⁻ in the above formula (9) is preferably an anion represented by the following formula (9-2-1) (i.e., an anion represented by R^{D1}—COO⁻, wherein R^{D1} is a phenyl group), an anion represented by the following formula (9-2-2) (i.e., an anion represented by Rⁿ—SO₃⁻, wherein R^{D1} is a group derived from 1,7,7-trimethyl bicyclo[2.2.1]heptan-2-one) or an anion represented by the following formula (9-2-3) (i.e., an anion represented by R^{D1}—N⁻—SO₂—R^{D2}, wherein R^{D1} is a butyl group, and R^{D2} is a trifluoromethyl group).

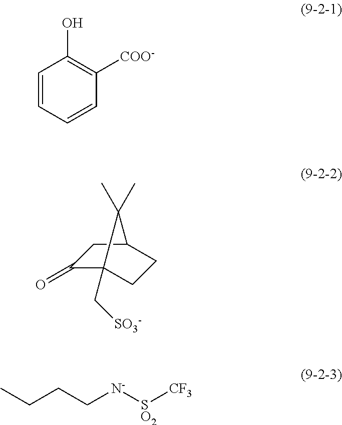

The aforementioned photodegradable acid diffusion controller is preferably a compound represented by the above formula (9), and more specifically, a sulfonium salt compound or an iodonium salt compound that meets the definition in the foregoing is preferred.

Examples of the sulfonium salt compound include triphenylsulfonium hydroxide, triphenylsulfonium salicylate, triphenylsulfonium 4-trifluoromethyl salicylate, diphenyl-4-hydroxyphenylsulfonium salicylate, triphenylsulfonium 10-camphorsulfonate, 4-t-butoxyphenyl diphenylsulfonium 10-camphorsulfonate, and the like. It is to be noted that these sulfonium salt compounds may be used either alone of one type, or in combination of two or more types thereof.

Further, examples of the iodonium salt compound include bis(4-t-butylphenyl)iodonium hydroxide, bis(4-t-butylphenyl)iodonium salicylate, bis(4-t-butylphenyl)iodonium 4-trifluoromethyl salicylate, bis(4-t-butylphenyl)iodonium 10-camphorsulfonate, and the like. It is to be noted that these iodonium salt compound may be used either alone of one type, or in combination of two or more types thereof.

It is to be noted that the acid diffusion controller (D) may be used either alone of one type, or in combination of two or more types thereof. The content of the acid diffusion control agent (D) is with respect to 100 parts by mass of the total amount of the polymer included in the radiation-sensitive resin composition is preferably no greater than 30 parts by mass, more preferably no greater than 20 parts by mass, still more preferably no greater than 10 parts by mass, and particularly preferably no greater than 5 parts by mass. When the acid diffusion control agent (D) is contained in an excessive amount, the resist coating film formed may have remarkably impaired sensitivity.

### <(E) Solvent>

The radiation-sensitive resin composition of the embodiment of the present invention typically contains (E) a solvent. The solvent (E) is not particularly limited as long as it is a solvent that can dissolve at least the polymer (A), the acid generating agent (B), and the polymer (C) contained as desired, and the like. Examples of the solvent (E) include

linear or branched ketones such as 2-pentanone, 2-hexanone, 2-heptanone, and 2-octanone;

cyclic ketones such as cyclopentanone, and cyclohexanone;

propylene glycol monoalkyl ether acetates such as propylene glycol monomethyl ether acetate, and propylene glycol monoethyl ether acetate;

ethylene glycol monoalkyl ether acetates such as ethylene glycol monomethyl ether acetate, and ethylene glycol monoethyl ether acetate;

propylene glycol monoalkyl ethers such as propylene glycol monomethyl ether, and propylene glycol monoethyl ether;

ethylene glycol monoalkyl ethers such as ethylene glycol monomethyl ether, and ethylene glycol monoethyl ether;

diethylene glycol dialkyl ethers such as diethylene glycol dimethyl ether, and diethylene glycol diethyl ether;

alkyl 2-hydroxypropionates such as methyl 2-hydroxypropionate, and ethyl 2-hydroxypropionate;

alkyl 3-alkoxypropionates such as methyl 3-methoxypropionate, ethyl 3-methoxypropionate, methyl 3-ethoxypropionate, and ethyl 3-ethoxypropionate;

esters such as n-butyl acetate, methyl pyruvate, and ethyl pyruvate; and the like.

Among these, linear or branched ketones, cyclic ketones, propylene glycol monoalkyl ether acetates, propylene glycol monoalkyl ethers, alkyl 2-hydroxypropionates and alkyl 3-alkoxypropionates are preferred, and of these, propylene glycol monomethyl ether acetate, propylene glycol monomethyl ether and cyclohexanone are more preferred. The solvent (E) may be used either alone of one type, or as a mixture of two or more types thereof.

### <(F) Additive>

Into the radiation-sensitive resin composition of the embodiment of the present invention may be blended in addition to the aforementioned components, an uneven distribution accelerator, a surfactant, an alicyclic compound, a sensitizing agent, a crosslinking agent, and the like as (F) additives if necessary.

(Uneven Distribution Accelerator)

The uneven distribution accelerator has an effect of allowing the polymer (A) to be unevenly distributed more efficiently in the surface of the resist film. When the uneven distribution accelerator is included in the radiation-sensitive resin composition, the amount of the polymer (A) added can be reduced as compared with conventional levels. Therefore, further suppression of elution of components from a resist film into a liquid immersion liquid, and carrying out liquid immersion lithography at a high speed by high speed scanning are enabled without deteriorating fundamental characteristics as a resist such as LWR, development defects, n pattern collapse resistance and the like. As a result, hydrophobicity of the surface of the resist film that inhibits defects derived from liquid immersion such as watermark defects can be enhanced. As an exemplary uneven distribution accelerator having such features, a low molecular compound having a relative permittivity of 30 or greater and no greater than 200, and a boiling point of at 1 atm (101.325 kPa) of no less than 100° C. may be used. Specific examples of such a compound include, lactone compounds, carbonate compounds, nitrile compounds, polyhydric alcohols, and the like.

Specific examples of the lactone compound described above include γ-butyrolactone, valerolactone, mevalonic lactone, norbornanelactone, and the like.

Specific examples of the carbonate compound described above include propylene carbonate, ethylene carbonate, butylene carbonate, vinylene carbonate, and the like.

Specific examples of the nitrile compound described above include succinonitrile, and the like. Specific examples of the polyhydric alcohol described above include glycerin, and the like.

In the radiation-sensitive resin composition of the embodiment of the present invention, the content of the uneven distribution accelerator with respect to 100 parts by mass of the total amount of the polymer is preferably 10 to 500 parts n by mass, and more preferably 30 to 300 parts by mass. The aforementioned uneven distribution accelerator may be contained only 1 type thereof, or two or more types thereof.

(Surfactant)

The surfactant is a component having actions of improving coating properties, developability, and the like. Examples of the surfactant include nonionic surfactants such as polyoxyethylene lauryl ether, polyoxyethylene stearyl ether, polyoxyethylene oleyl ether, polyoxyethylene n-octylphenyl ether, polyoxyethylene n-nonylphenyl ether, polyethylene glycol dilaurate and polyethylene glycol distearate, as well as trade names KP341 (manufactured by Shin-Etsu Chemical Co., Ltd.), Polyflow No. 75 and Polyflow No. 95 (manufactured by Kyoeisha Chemical Co., Ltd.), EFTOP EF301, EFTOP EF303 and EFTOP EF352 (manufactured by Tochem Products Corporation), Megaface® F171 and Megaface® F173 (manufactured by Dainippon Ink And Chemicals, Incorporated), Fluorad™ FC430 and Fluorad™ FC431 (manufactured by Sumitomo 3M Limited), ASAHI GUARD AG710, Surflon S-382, Surflon SC-101, Surflon SC-102, Surflon SC-103, Surflon SC-104, Surflon SC-105 and Surflon SC-106 (manufactured by Asahi Glass Co., Ltd.), and the like. These surfactants may be used either alone of one type, or as a mixture of at least two types thereof. The content of the aforementioned surfactant with respect to 100 parts by mass of the total amount of the polymer included in the radiation-sensitive resin composition is typically no greater than 2 parts by mass.

(Compound Having Alicyclic Skeleton)

The alicyclic skeleton-containing compound is a component that exhibits actions of further improving the dry etching resistance, pattern configuration, adhesiveness to a substrate, and the like. Examples of the alicyclic skeleton-containing compound include

adamantane derivatives such as 1-adamantanecarboxylic acid, 2-adamantanone and t-butyl 1-adamantanecarboxylate;

deoxycholic acid esters such as t-butyl deoxycholate, t-butoxycarbonylmethyl deoxycholate and 2-ethoxyethyl deoxycholate;

lithocholic acid esters such as t-butyl lithocholate, t-butoxycarbonylmethyl lithocholate and 2-ethoxyethyl lithocholate;

3-[2-hydroxy-2,2-bis(trifluoromethyl)ethyl]tetracyclo[4.4.0.1^{2,5.} 1^{7,10}]dodecane, 2-hydroxy-9-methoxycarbonyl-5-oxo-4-oxa-tricyclo[4.2.1.0^{3,7}]nonane, and the like. These alicyclic skeleton-containing compounds may be used either alone of one type, or as a mixture of two or more thereof. These alicyclic skeleton-containing compounds may be used either alone of one type, or as a mixture of two or more thereof. The content of the aforementioned compound having an alicyclic skeleton with respect to 100 parts by mass of the total amount of the polymer included in the radiation-sensitive resin composition is typically no greater than 50 parts by mass, and preferably no greater than 30 parts by mass.

(Sensitizing Agent)

The sensitizer serves in absorbing the energy other than the energy of radioactive rays absorbed to the acid generating agent (B) and the like, and transferring the energy to the acid generator (B) and the like in the form of, for example, radicals, thereby increasing the amount of acid generation, and thus has an effect of improving “apparent sensitivity” of the radiation-sensitive resin composition.

Examples of the sensitizing agent include carbazoles, acetophenones, benzophenones, naphthalenes, phenols, biacetyl, eosin, rose bengal, pyrenes, anthracenes, phenothiazines, and the like. These sensitizing agents may be used either alone of one type, or as a mixture of at least two types thereof.

(Crosslinking Agent)

When the radiation-sensitive resin composition of the embodiment of the present invention is used as a negative type radiation-sensitive resin composition, a compound that enables in the presence of an acid, crosslinking of a polymer that is soluble in an alkaline developer (hereinafter, may be also referred to as “crosslinking agent”) may be also blended. The crosslinking agent is exemplified by compounds having one or more types of functional groups having crosslinking reactivity with the polymer that is soluble in an alkaline developer (hereinafter, referred to as “crosslinkable functional group”).

Examples of the crosslinkable functional group include glycidyl ether group, a glycidyl ester group, a glycidylamino group, a methoxymethyl group, an ethoxymethyl group, a benzyloxy methyl group, an acetoxy methyl group, a benzoyloxy methyl group, a formyl group, an acetyl group, a vinyl group, an isopropenyl group, a (dimethylamino)methyl group, a (diethylamino)methyl group, a (dimethylolamino)methyl group, a (diethylolamino)methyl group, a morpholinomethyl group, and the like.

The crosslinking agent is exemplified by those described in paragraphs nos. [0169] to [0172] of PCT International Publication No. 2009/51088.

As the crosslinking agent described above, particularly, methoxymethyl group-containing compounds, and more specifically, dimethoxymethylurea and tetramethoxymethylglycoluril are preferred. In the radiation-sensitive negative type resin composition, the crosslinking agent may be used either alone or as a mixture of two or more thereof.

The content of the crosslinking agent with respect to 100 parts by mass of the polymer that is soluble in an alkaline developer is preferably 5 to 95 parts by mass, more preferably 15 to 85 parts by mass, and particularly preferably 20 to 75 parts by mass. When the content of the crosslinking agent is less than 5 parts by mass, a decrease in the percentage of residual film, as well as meandering, swelling, etc., of the pattern are likely to occur. To the contrary, when the content exceeds 95 parts by mass, the alkali developability is likely to be decreased.

In addition to the aforementioned additives, a dye, a pigment, an adhesion promoter and the like may be used as the additive (F). For example, use of a dye or pigment enables a latent image at a light-exposed site to be visualized, whereby influences of halation upon exposure can be mitigated. Moreover, when an adhesion promoter is blended, the adhesiveness to a substrate can be improved. As the other additive, an alkali-soluble resin, a low molecular alkali-soluble controlling agent having an acid-dissociable protecting group, a halation inhibitor, a storage stabilizing agent, a defoaming agent, and the like may be included.

It is to be noted that the additive (F) may be used either one type alone of various types of additives described in the foregoing, or two or more thereof may be used in combination.

### <Preparation Method of a Radiation-Sensitive Resin Composition>

The radiation-sensitive resin composition of the embodiment of the present invention is generally prepared by dissolving in the solvent (E) so as to give the total solid content of usually 1 to 50% by mass, and preferably 1 to 25% by mass, followed by filtration with a filter having a pore size of, for example, about 5 nm. The material of the filter is not particularly limited, and for example, nylon 6,6, nylon 6, polyethylene, a combination of these, or the like may be included.

It is to be noted that the content of impurities such as halogen ion and metals in the radiation-sensitive resin composition is preferably as low as possible. When the content of such impurities is small, sensitivity, resolution, process stability, pattern configuration and the like of the resist coating film can be further improved. Therefore, polymers such as the polymer (A) and the polymer (C) included in the radiation-sensitive resin composition are preferably purified by, for example, washing with water, a chemical purification method such as liquid-liquid extraction, a combined method of such a chemical purification method with a physical purification such as ultrafiltration and centrifugal separation, and the like.

### <Formation Method of a Resist Pattern>

The method for forming a resist pattern of the embodiment of the present invention includes: (1) a step of forming a photoresist film on a substrate using the radiation-sensitive resin composition (hereinafter, may be also referred to as “step (1)”), (2) a step of subjecting the photoresist film to liquid immersion lithography through a liquid for immersion lithography disposed on the photoresist film (hereinafter, may be also referred to as “step (2)”), and (3) a step of forming a resist pattern by developing the photoresist film subjected to the liquid immersion lithography (hereinafter, may be also referred to as “step (3)”). According to such a formation method, formation of a resist pattern having a favorable pattern configuration is enabled.

In the step (1), a photoresist film is formed by coating a solution of the radiation-sensitive resin composition of the embodiment of the present invention on a substrate such as, for example, a silicon wafer, or a wafer coated with aluminum by an appropriate coating means such as means of spin coating, cast coating or roll coating. Specifically, after a solution of the radiation-sensitive resin composition is coated such that the resulting resist film has a predetermined film thickness, prebaking (PB) is carried out to allow the solvent in the coating film to be volatilized, whereby a resist film is formed.

The thickness of the resist film is not particularly limited, and is preferably 10 to 5,000 nm, and more preferably 10 to 2,000 nm.

Also, conditions of heating in the prebaking may vary depending on the blend composition of the radiation-sensitive resin composition, and may involve preferably about 30 to 200° C. and more preferably 50 to 150° C.

In the step (2), a liquid for immersion lithography is provided on the photoresist film formed in the step (1), and a radioactive ray is irradiated through the liquid for immersion lithography to execute liquid immersion lithography of the photoresist film.

As the liquid for immersion lithography, for example, pure water, long chain or cyclic aliphatic compound or the like may be used.

The radioactive ray employed is appropriately selected from visible light rays, ultraviolet rays, far ultraviolet rays, X-rays, charged particle rays and the like in accordance with the type of the acid generator used. The radioactive ray is preferably a far ultraviolet ray typified by an ArF excimer laser (wavelength: 193 nm) or a KrF excimer laser (wavelength: 248 nm), and particularly preferably an ArF excimer laser (wavelength: 193 nm).

Also, conditions of the exposure such as exposure dose may be appropriately determined in accordance with the blend composition of the radiation-sensitive resin composition and the type of the additives.

In the embodiment of the present invention, a heat treatment (PEB: post exposure baking) is preferably carried out after the exposure. The PEB enables a dissociation reaction of the acid-dissociable group in the resin components to smoothly proceed. Conditions of heating of the PEB may be appropriately adjusted depending on the blend composition of the radiation-sensitive resin composition, and involve usually 30 to 200° C., and preferably 50 to 170° C.

In the embodiment of the present invention, in order to maximize the potential capability of the radiation-sensitive resin composition, an organic or inorganic antireflection film may be also formed on the substrate employed, as disclosed in, for example, Japanese Examined Patent, Publication No. H6-12452 (Japanese Unexamined Patent Application, Publication No. S59-93448), and the like. Moreover, in order to prevent influences of basic impurities etc., included in the environment atmosphere, a protective film may be also provided on the photoresist film, as disclosed in, for example, Japanese Unexamined Patent Application, Publication No. H5-188598, and the like. Furthermore, in order to prevent effluence of the acid generator etc., from the photoresist film during the liquid immersion lithography, a protective film for liquid immersion may be provided on the photoresist film, as disclosed in, for example, Japanese Unexamined Patent Application, Publication No. 2005-352384, and the like. It is to be noted that these techniques may be used in combination.

In the method for forming a resist pattern by the liquid immersion lithography, the resist pattern can be formed with only the photoresist film obtained using the radiation-sensitive resin composition of the embodiment of the present invention, without providing the protective film (upper layer film) described above on the photoresist film. If a resist pattern is formed with the photoresist film that is free from the upper layer film, a step of forming a protective film (upper layer film) can be omitted, thereby capable of leading to expectation for improvement of throughput.

In the step (3), a predetermined resist pattern is formed by subjecting the exposed resist film to development.

Examples of preferable developer solution used in the development process include aqueous alkali solutions prepared by dissolving at least one alkaline compound such as sodium hydroxide, potassium hydroxide, sodium carbonate, sodium silicate, sodium metasilicate, ammonia water, ethylamine, n-propylamine, diethylamine, di-n-propylamine, triethylamine, methyldiethylamine, ethyldimethylamine, triethanolamine, tetramethylammonium hydroxide, pyrrole, piperidine, choline, 1,8-diazabicyclo-[5.4.0]-7-undecene or 1,5-diazabicyclo-[4.3.0]-5-nonene.

The concentration of the alkaline aqueous solution is preferably no greater than 10% by mass. In the case in which the concentration of the alkaline aqueous solution is greater than 10% by mass, sites unexposed with light may be also dissolved in the developing solution.

In addition, an organic solvent may be also added to the n developing solution consisting of the aforementioned alkaline aqueous solution.

Examples of the organic solvent include ketones such as acetone, methyl ethyl ketone, methyl-1-butyl ketone, cyclopentanone, cyclohexanone, 3-methyl cyclopentanone and 2,6-dimethyl cyclohexanone; alcohols such as methyl alcohol, ethyl alcohol, n-propyl alcohol, i-propyl alcohol, n-butyl alcohol, t-butyl alcohol, cyclopentanol, cyclohexanol, 1,4-hexanediol and 1,4-hexanedimethylol; ethers such as tetrahydrofuran and dioxane; esters such as ethyl acetate, n-butyl acetate and i-amyl acetate; aromatic hydrocarbons such as toluene and xylene, as well as phenol, acetonyl acetone, dimethylformamide, and the like. These organic solvents may be used either alone, or two or more types thereof may be used in combination.

The amount of the organic solvent used is preferably no greater than 100 parts by volume with respect to 100 parts by volume of the alkaline aqueous solution. In the case in which the amount of the organic solvent used is greater than 100 parts by volume, developability is lowered, and thus undeveloped portion at the site exposed with light may increase. Moreover, to the developing solution consisting of the alkaline aqueous solution may be added an appropriate amount of a surfactant and the like. It is to be noted that the development with a developing solution consisting of the alkaline aqueous solution is, in general, followed by washing with water and drying.

According to the resist pattern obtained as described above using the radiation-sensitive resin composition of the embodiment of the present invention, deterioration of coating film performances due to elution from the resist coating film is suppressed, and occurrence of various types of defects such as watermark defects, bubble defects and development defects is inhibited; therefore, favorable patterning characteristics are achieved, and thus the radiation-sensitive resin composition of the embodiment of the present invention is suited for microfabrication carried out using a lithography technique.

### EXAMPLES

Hereinafter, the present invention is explained in detail by way of Examples, but the present invention is not limited thereto. Measuring methods of various types of physical property values are shown below.

### [Weight Average Molecular Weight (Mw), and Number Average Molecular Weight (Mn)]

Using GPC columns (G2000HXL×2, G3000HXL×1, and G4000HXL×1) manufactured by Tosoh Corporation, gel permeation chromatography (GPC) was carried out under analysis conditions including a flow rate of 1.0 ml/min, an elution solvent of tetrahydrofuran, and a column temperature of 40° C., with mono-dispersed polystyrene as a standard.

### [¹H-NMR Analysis, ¹³C-NMR Analysis]

A ¹H-NMR analysis of the compound, and a ¹³C-NMR analysis for determination of the content of fluorine atoms of the polymer were carried out using a nuclear magnetic resonance apparatus (manufactured by JEOL, Ltd. “JNM-ECX400”).

### Synthesis of Compound (i)

### Example 1

### Synthesis of 3-(2,2,2-trifluoroacetoxy)-1-adamantyl methacrylate

After a reaction vessel which had been sufficiently dried inside by vacuum heating was replaced with dry nitrogen, 23.63 g (0.1 mol) of 3-hydroxyadamantyl methacrylate, 23.10 g (0.11 mol) of trifluoroacetic anhydride and 500 mL of THF were added into the reaction vessel. Thereafter, the mixture was stirred at room temperature for 2 hrs. Subsequently, 300 g of a saturated aqueous sodium bicarbonate solution and 500 mL of ethyl acetate were added thereto, and then the organic layer was separated to obtain an extraction liquid. This extraction liquid was washed with a saturated saline solution, and thereafter dried by adding anhydrous sodium sulfate (drying agent). Thereafter, the drying agent was filtered off with a Buechner funnel, and then the organic solvent was distilled off and the residue was purified on silica gel column chromatography. Accordingly, 3-(2,2,2-trifluoroacetoxy)adamantyl methacrylate (30.95 g (yield: 93%)) represented by the following formula (M-18) was obtained.

Note that the ¹H-NMR data of 3-(2,2,2-trifluoroacetoxy)adamantyl methacrylate obtained in Example 1 are shown below.

¹H-NMR (CDCl₃)δ: 1.53-1.69 (m, 2H), 1.80-1.94 (m, 3H), 2.07-2.26 (m, 8H), 2.39-2.50 (m, 2H), 2.52-2.67 (m, 2H), 5.52 (s, 1H, C═CH₂), 6.02 (s, 1H, C═CH₂)

### Example 2

### Synthesis of 3-(methacryloyloxy)-1-adamantyl-2,2,3,3,4,4,4-heptafluorobutanoate

After a reaction vessel which had been sufficiently dried inside by vacuum heating was replaced with dry nitrogen, 23.63 g (0.1 mol) of 3-hydroxyadamantyl methacrylate, 45.11 g (0.11 mol) of heptafluorobutyric anhydride and 500 mL of THF were added into the reaction vessel. Thereafter, the mixture was stirred at room temperature for 2 hrs. Subsequently, 300 g of a saturated aqueous sodium bicarbonate solution and 500 mL of ethyl acetate were added thereto, and then the organic layer was separated to obtain an extraction liquid. This extraction liquid was washed with a saturated saline solution, and thereafter dried using anhydrous sodium sulfate (drying agent). Thereafter, the drying agent was filtered off with a Buechner funnel, and then the organic solvent was distilled off and the residue was purified on silica gel column chromatography. Accordingly, (methacryloyloxy)-1-adamantyl-2,2,3,3,4,4,4-heptafluorobutanoate (35.87 g (yield: 83%)) represented by the following formula (M-19) was obtained.

Note that the ¹H-NMR data of 3-(methacryloyloxy)-1-adamantyl-2,2,3,3,4,4,4-heptafluorobutanoate obtained in Example 2 are shown below.

¹H-NMR (CDCl₃)δ: 1.51-1.66 (m, 2H), 1.81-1.96 (m, 3H), 2.07-2.30 (m, 8H), 2.37-2.48 (m, 2H), 2.49-2.66 (m, 2H), 5.51 (s, 1H, C═CH₂), 6.02 (s, 1H, C═CH₂)

### Example 3

### Synthesis of (1-(2,2,2-trifluoroacetoxy)adamantyl)methyl methacrylate

After a reaction vessel which had been sufficiently dried inside by vacuum heating was replaced with dry nitrogen, 25.03 g (0.1 mol) of 1-hydroxyadamantylmethyl methacrylate, 23.10 g (0.11 mol) of trifluoroacetic anhydride and 500 mL of THF were added into the reaction vessel. Thereafter, the mixture was stirred at room temperature for 2 hrs. Subsequently, 300 g of a saturated aqueous sodium bicarbonate solution and 500 mL of ethyl acetate were added thereto, and then the organic layer was separated to obtain an extraction liquid. This extraction liquid was washed with a saturated saline solution, and thereafter dried using anhydrous sodium sulfate (drying agent). Thereafter, the drying agent was filtered off with a Buechner funnel, and then the organic solvent was distilled off and the residue was purified on silica gel column chromatography. Accordingly, (1-(2,2,2-trifluoroacetoxy)-1-adamantyl)methyl methacrylate (31.50 g (yield: 91%)) represented by the following formula (M-20) was obtained.

Note that the ¹H-NMR data of (1-(2,2,2-trifluoroacetoxy)adamantyl)methyl methacrylate obtained in Example 3 are shown below.

¹H-NMR (CDCl₃)δ: 1.47-1.72 (m, 2H), 1.79-1.91 (m, 3H), 1.98-2.33 (m, 8H), 2.41-2.56 (m, 2H), 2.61-2.78 (m, 2H), 4.22 (d, 2H), 5.54 (s, 1H, C═CH₂), 6.00 (s, 1H, C═CH₂)

### Example 4

### Synthesis of (2-oxo-2-(3-(2,2,2-trifluoroacetoxy)-1-adamantyloxy)ethyl methacrylate

After a reaction vessel which had been sufficiently dried inside by vacuum heating was replaced with dry nitrogen, 29.43 g (0.1 mol) of 3-hydroxyadamantyl methacrylate, 23.10 g (0.11 mol) of trifluoroacetic anhydride and 500 mL of THF were added into the reaction vessel. Thereafter, the mixture was stirred at room temperature for 2 hrs. Subsequently, 300 g of a saturated aqueous sodium bicarbonate solution and 500 mL of ethyl acetate were added thereto, and then the organic layer was separated to obtain an extraction liquid. This extraction liquid was washed with a saturated saline solution, and thereafter dried using anhydrous sodium sulfate (drying agent). Thereafter, the drying agent was filtered off with a Buechner funnel, and then the organic solvent was distilled off and the residue was purified on silica gel column chromatography. Accordingly, 2-oxo-2-(3-(2,2,2-trifluoroacetoxy)-1-adamantyloxy)ethyl methacrylate (33.56 g (yield: 86%)) represented by the following formula (M-21) was obtained.

Note that the ¹H-NMR data of 3-(2,2,2-trifluoroacetoxy)adamantyl methacrylate obtained in Example 4 are shown below.

¹H-NMR (CDCl₃)δ: 1.50-1.67 (m, 2H), 1.79-1.98 (m, 3H), 2.00-2.22 (m, 8H), 2.35-2.49 (m, 2H), 2.50-2.65 (m, 2H), 5.11 (d, 2H), 5.50 (s, 1H, C═CH₂), 6.01 (s, 1H, C═CH₂)

### Example 5

### Synthesis of 3,5-di(trifluoroacetoxy)-1-adamantyl methacrylate

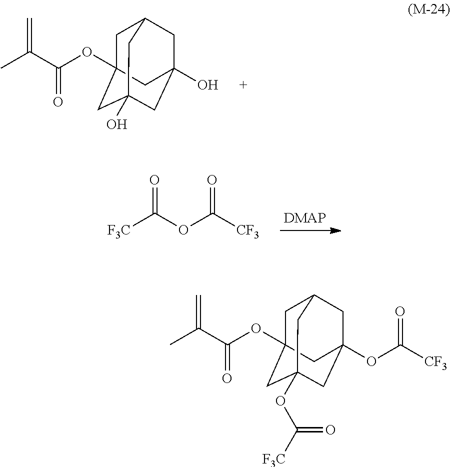 [SMILES: C=C(C)C(=O)OC12CC3CC(O)(CC(O)(C3)C1)C2 ; O=C(OC(=O)C(F)(F)F)C(F)(F)F ; C=C(C)C(=O)OC12CC3CC(OC(=O)C(F)(F)F)(C1)CC(OC(=O)C(F)(F)F)(C3)C2]

After a reaction vessel which had been sufficiently dried inside by vacuum heating was replaced with dry nitrogen, a stirring bar, 5.00 g (0.0198 mol) of 3,5-dihydroxy-1-adamantyl methacrylate represented by the above formula and 0.121 g (0.00099 mol) of dimethylamino pyridine (DMAP) were added into the reaction vessel, and the mixture was stirred using a stirrer while cooling the reaction vessel in an ice bath. Thereto was added 12.50 g (0.0594 mol) of trifluoroacetic anhydride dropwise over 5 min, and the mixture was stirred for 10 min in the ice bath. Thereafter, the mixture was stirred at room temperature for 10 hrs. Subsequently, 100 g of a saturated aqueous sodium bicarbonate solution and 200 mL of ethyl acetate were added thereto, and then the organic layer was separated to obtain an extraction liquid. This extraction liquid was washed with a saturated saline solution, and thereafter dried over anhydrous sodium sulfate (drying agent). Thereafter, the drying agent was filtered off with a Buechner funnel, and then the organic solvent was distilled off and the residue was purified on silica gel column chromatography. Accordingly, 3,5-di(trifluoroacetoxy)-1-adamantyl methacrylate (4.51 g (yield: 51%)) represented by the formula (M-24) was obtained.

Note that the ¹H-NMR data of 3,5-di(trifluoroacetoxy)-1-adamantyl methacrylate obtained in Example 5 are shown below.

¹H-NMR (CDCl₃)δ: 1.85-1.95 (m, 3H), 2.10-2.25 (m, 6H), 2.55-2.75 (m, 7H), 5.56 (s, 1H, C═CH₂), 6.04 (s, 1H, C═CH₂)

### Synthesis of Polymer (A) and Polymer (C)

Using each compound (i) (compounds represented by the following formulae (M-18) to (M-21) and (M-24)) synthesized as described in the foregoing, and a compound selected from other compounds (compounds represented by the following formulae (M-1) to (M-17), (M-22), (M-23) and (M-25)), polymers (A-1) to (A-21) as the polymer (A), and polymers (C-1) to (C-5) as the polymer (C) were synthesized according to the following method.

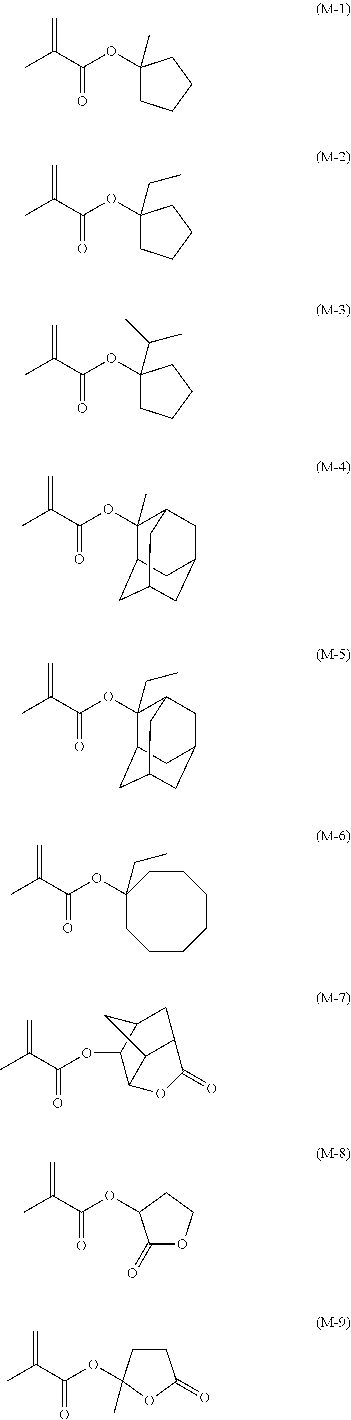

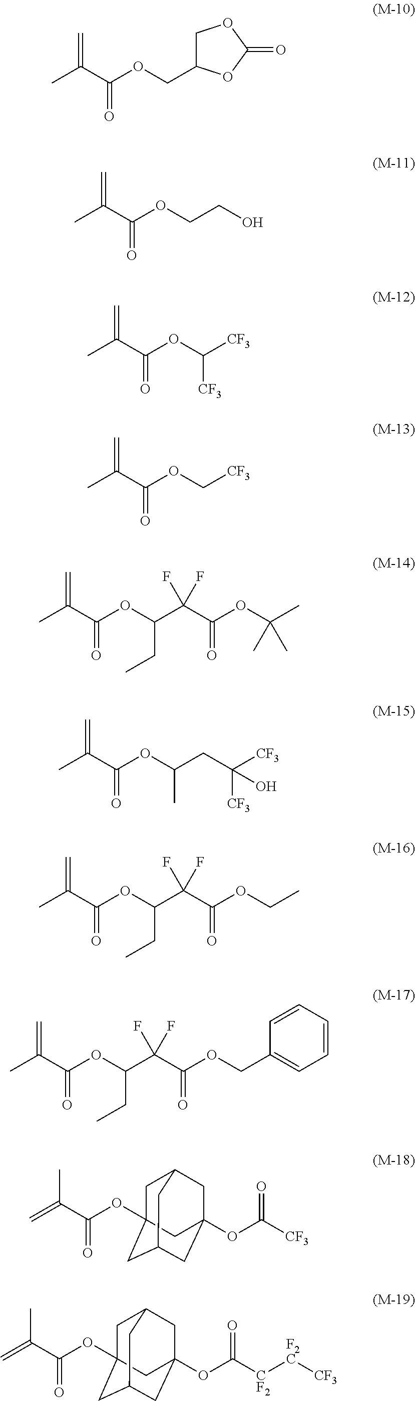

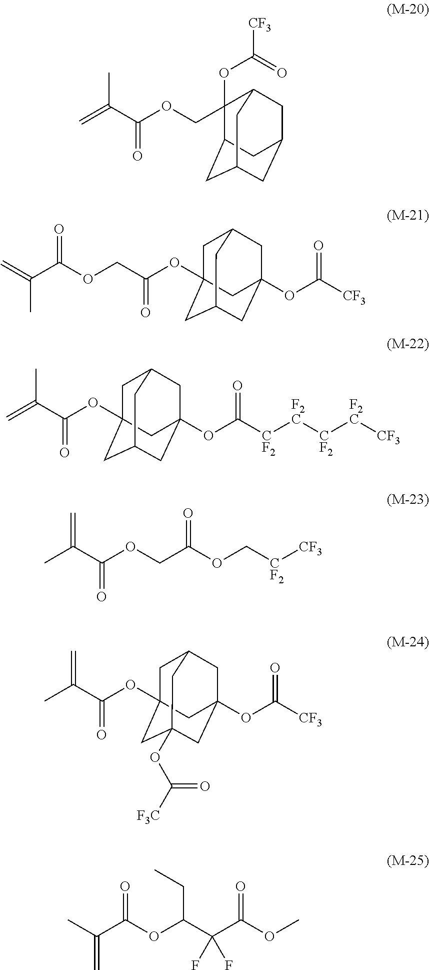 [SMILES: C=C(C)C(=O)OCC1(OC(=O)C(F)(F)F)C2CC3CC(C2)CC1C3 ; C=C(C)C(=O)OCC(=O)OC12CC3CC(C1)CC(OC(=O)C(F)(F)F)(C3)C2 ; C=C(C)C(=O)OC12CC3CC(C1)CC(OC(=O)C(F)(F)C(F)(F)C(F)(F)C(F)(F)C(F)(F)F)(C3)C2 ; C=C(C)C(=O)OCC(=O)OCC(F)(F)C(F)(F)F ; C=C(C)C(=O)OC12CC3CC(OC(=O)C(F)(F)F)(C1)CC(OC(=O)C(F)(F)F)(C3)C2 ; C=C(C)C(=O)OC(CC)C(F)(F)C(=O)OC]

### Synthesis of the Polymer (A)

### Example 6

### Synthesis of Polymer (A-1)

The compound (M-18) in an amount of 5.0 g (0.0150 mol) was dissolved in 10 g of 2-butanone, and therewith 0.25 g of dimethyl 2,2′-azobis(2-methylpropionate) was charged into a 200 mL three-neck flask. After the reactor vessel was purged with nitrogen for 30 min, it was heated to 80° C. while stirring the mixture. The time point at which the heating was started was defined as a polymerization starting time, and the polymerization reaction was performed for 4 hrs. After completing the polymerization, the polymerization solution was cooled to no higher than 30° C. by water cooling. The polymerization solution was concentrated in vacuo with an evaporator until the weight of the polymerization solution became 7.5 g. Thereafter, thus concentrated liquid was charged into a mixed liquid of 50 g of methanol and 50 g of water to allow a slime white solid to be precipitated. The liquid portion was eliminated by decantation, and again the mixed liquid of 50 g of methanol and 50 g of water was charged, whereby the slime white solid was repeatedly washed twice. The recovered solid was dried in vacuo at 60° C. for 15 hrs to give 3.1 g of a white powdery polymer (A-1) (yield: 62%). The polymer (A-1) had the Mw of 6,900, and the Mw/Mn of 1.44. As a result of a ¹³C-NMR analysis, the content of a structural unit derived from the compound (M-18) was 100 mol %.

### Example 7

### Synthesis of Polymer (A-2)

A monomer solution was prepared by dissolving 4.0 g (0.0120 mol, 80 mol %) of the compound (M-18) and 0.59 g (0.0030 mol, 20 mol %) of the compound (M-3) in 10 g of 2-butanone, and further dissolving 0.90 g of dimethyl 2,2′-azobis(2-methylpropionate). On the other hand, 5 g of 2-butanone was charged into a 100 mL three-neck flask, and the reactor vessel was purged with nitrogen for 30 min, followed by heating to 80° C. while stirring the mixture. Next, the monomer solution prepared beforehand was added dropwise over 3 hrs using a dripping funnel. The time point at which the dropwise addition was started was defined as a polymerization starting time, and the polymerization reaction was performed for 6 hrs.

After completing the polymerization, the polymerization solution was cooled to no higher than 30° C. by water cooling. The polymerization solution was concentrated in vacuo with an evaporator until the weight of the polymerization solution became 30 g. Thereafter, thus concentrated liquid was charged into a mixed liquid of 100 g of methanol and 100 g of water to allow a slime white solid to be precipitated. The liquid portion was eliminated by decantation, and 100 g of methanol was charged, whereby the slime white solid was repeatedly washed twice. The recovered solid was dried in vacuo at 60° C. for 15 hrs to give 15.1 g of a white powdery polymer (A-2) (yield: 76%). The polymer (A-2) had the Mw of 4,900, and the Mw/Mn of 1.39. As a result of a ¹³C-NMR analysis, each content of structural units derived from the compounds (M-18) and (M-3) was 81.4 mol % and 18.6 mol %.

### Examples 8 to 26

### Synthesis of Polymers (A-3) to (A-21)

Polymers (A-3) to (A-21) were prepared in a similar manner to Example 7 except that the total number of moles of the monomer compound became identical (0.0153 mol), and that each charging amount (molar ratio) in the polymerization reaction was as shown in Tables 1-1 and 1-2. Physical property values of the polymers (A-1) to (A-21) are shown in Tables 2-1 and 2-2.

### Synthesis Examples 1 to 8

### Synthesis of Polymers (a-1) to (a-8)

In addition, polymers (a-1) to (a-8) were prepared in a similar manner to Example 7 without using the compound (i), but using the compound as shown in Tables 1-1 and 1-2. Physical property values of the polymers (a-1) to (a-8) are also shown in Tables 2-1 and 2-2.

TABLE 1-1

| | | Monomer compound and Amount charged | | | | | | | | | |
|---|---|---|---|---|---|---|---|---|---|---|---|
| | | Structural | | Structural units | | Structural | | Structural | | Other | |
| | (A) | unit (I) | | (II) and (III) | | unit (IV) | | unit (VI) | | structural unit | |
| | Component | type | mol % | type | mol % | type | mol % | type | mol % | type | mol % |
| Example 6 | A-1 | M-18 | 100 | — | — | — | — | — | — | — | — |
| Example 7 | A-2 | M-18 | 80 | — | — | M-3 | 20 | — | — | — | — |
| Example 8 | A-3 | M-19 | 80 | — | — | M-3 | 20 | — | — | — | — |
| Example 9 | A-4 | M-20 | 80 | — | — | M-3 | 20 | — | — | — | — |
| Example 10 | A-5 | M-21 | 80 | — | — | M-3 | 20 | — | — | — | — |
| Example 11 | A-6 | M-18 | 80 | — | — | M-5 | 20 | — | — | — | — |
| Example 12 | A-7 | M-18 | 80 | — | — | M-6 | 20 | — | — | — | — |
| Example 13 | A-8 | M-18 | 90 | — | — | — | — | — | — | M-11 | 10 |
| Example 14 | A-9 | M-18 | 90 | M-12 | 10 | — | — | — | — | — | — |
| Example 15 | A-10 | M-18 | 90 | M-13 | 10 | — | — | — | — | — | — |
| Example 16 | A-11 | M-18 | 80 | M-14 | 20 | — | — | — | — | — | — |
| Example 17 | A-12 | M-18 | 80 | M-15 | 20 | — | — | — | — | — | — |
| Example 18 | A-13 | M-18 | 80 | M-16 | 20 | — | — | — | — | — | — |
| Example 19 | A-14 | M-18 | 80 | M-17 | 20 | — | — | — | — | — | — |
| Example 20 | A-15 | M-18 | 20 | M-16 | 50 | M-3 | 30 | — | — | — | — |
| Example 21 | A-16 | M-18 | 70 | — | — | M-5 | 20 | — | — | M-11 | 10 |
| Example 22 | A-17 | M-18 | 70 | — | — | M-6 | 20 | M-7 | 10 | — | — |
| Example 23 | A-18 | M-24 | 80 | — | — | M-2 | 20 | — | — | — | — |
| Example 24 | A-19 | M-24 | 50 | — | — | M-6 | 50 | — | — | — | — |
| Example 25 | A-20 | M-24 | 10 | M-25 | 40 | M-6 | 50 | — | — | — | — |
| Example 26 | A-21 | M-24 | 40 | — | — | M-6 | 50 | M-10 | 10 | — | — |

TABLE 1-2

| | | Monomer compound and Amount charged | | | | | | | | | |
|---|---|---|---|---|---|---|---|---|---|---|---|
| | | Structural | | Structural units | | Structural | | Structural | | Other | |
| | (A) | unit (I) | | (II) and (III) | | unit (IV) | | unit (VI) | | structural unit | |
| | Component | type | mol % | type | mol % | type | mol % | type | mol % | type | mol % |
| Synthesis | a-1 | — | — | M-13 | 30 | M-2 | 70 | — | — | — | — |
| Example 1 | | | | | | | | | | | |
| Synthesis | a-2 | — | — | M-12 | 40 | — | — | M-8 | 30 | — | — |
| Example 2 | | | | M-15 | 30 | | | | | | |
| Synthesis | a-3 | — | — | M-12 | 40 | — | — | M-9 | 30 | — | — |
| Example 3 | | | | M-15 | 30 | | | | | | |
| Synthesis | a-4 | — | — | M-12 | 40 | M-2 | 30 | M-8 | 30 | — | — |
| Example 4 | | | | | | | | | | | |
| Synthesis | a-5 | — | — | — | — | — | — | — | — | M-22 | 100 |
| Example 5 | | | | | | | | | | | |
| Synthesis | a-6 | — | — | — | — | M-2 | 30 | — | — | M-22 | 70 |
| Example 6 | | | | | | | | | | | |
| Synthesis | a-7 | — | — | — | — | — | — | — | — | M-23 | 100 |
| Example 7 | | | | | | | | | | | |
| Synthesis | a-8 | — | — | — | — | M-2 | 30 | — | — | M-23 | 70 |
| Example 8 | | | | | | | | | | | |

TABLE 2-1

| | | Each structural unit and Content | | | | | | | | | | | | |
|---|---|---|---|---|---|---|---|---|---|---|---|---|---|---|
| | | Structural | | Structural units | | Structural | | Structural | | Other | | | | Fluorine |
| | (A) | unit (I) | | (II) and (III) | | unit (IV) | | unit (VI) | | structural unit | | | | atom content |
| | Component | type | mol % | type | mol % | type | mol % | type | mol % | type | mol % | Mw | Mw/Mn | (% by mass) |
| Example 6 | A-1 | M-18 | 100.0 | — | — | — | — | — | — | — | — | 6,900 | 1.44 | 17.15 |
| Example 7 | A-2 | M-18 | 81.4 | — | — | M-3 | 18.6 | — | — | — | — | 7,600 | 1.42 | 13.70 |
| Example 8 | A-3 | M-19 | 80.5 | — | — | M-3 | 19.5 | — | — | — | — | 8,100 | 1.48 | 24.60 |
| Example 9 | A-4 | M-20 | 81.9 | — | — | M-3 | 18.1 | — | — | — | — | 7,200 | 1.46 | 13.20 |
| Example 10 | A-5 | M-21 | 80.9 | — | — | M-3 | 19.1 | — | — | — | — | 7,400 | 1.53 | 11.70 |
| Example 11 | A-6 | M-18 | 81.5 | — | — | M-5 | 18.5 | — | — | — | — | 8,200 | 1.46 | 13.70 |
| Example 12 | A-7 | M-18 | 79.6 | — | — | M-6 | 20.4 | — | — | — | — | 8,700 | 1.48 | 13.70 |
| Example 13 | A-8 | M-18 | 88.5 | — | — | — | — | — | — | M-11 | 11.5 | 7,900 | 1.42 | 15.40 |
| Example 14 | A-9 | M-18 | 90.9 | M-12 | 9.1 | — | — | — | — | — | — | 8,200 | 1.45 | 15.40 |
| Example 15 | A-10 | M-18 | 90.3 | M-13 | 9.7 | — | — | — | — | — | — | 8,600 | 1.51 | 18.80 |
| Example 16 | A-11 | M-18 | 80.4 | M-14 | 19.6 | — | — | — | — | — | — | 8,300 | 1.43 | 16.50 |
| Example 17 | A-12 | M-18 | 78.2 | M-15 | 21.8 | — | — | — | — | — | — | 7,900 | 1.47 | 13.70 |
| Example 18 | A-13 | M-18 | 79.6 | M-16 | 20.4 | — | — | — | — | — | — | 8,300 | 1.52 | 16.80 |
| Example 19 | A-14 | M-18 | 78.7 | M-17 | 21.3 | — | — | — | — | — | — | 7,400 | 1.56 | 16.20 |
| Example 20 | A-15 | M-18 | 58.2 | M-16 | 22.5 | M-3 | 19.3 | — | — | — | — | 8,300 | 1.49 | 11.00 |
| Example 21 | A-16 | M-18 | 69.5 | — | — | M-5 | 18.1 | — | — | M-11 | 12.4 | 8,100 | 1.45 | 12.00 |
| Example 22 | A-17 | M-18 | 51.2 | — | — | M-6 | 21.2 | M-7 | 27.6 | — | — | 7,700 | 1.53 | 12.00 |
| Example 23 | A-18 | M-24 | 79.5 | — | — | M-2 | 20.5 | — | — | — | — | 7,000 | 1.41 | 23.20 |
| Example 24 | A-19 | M-24 | 50.9 | — | — | M-6 | 49.1 | — | — | — | — | 8,100 | 1.50 | 17.25 |
| Example 25 | A-20 | M-24 | 10.1 | M-25 | 40.4 | M-6 | 49.5 | — | — | — | — | 6,900 | 1.47 | 10.69 |
| Example 26 | A-21 | M-24 | 40.5 | — | — | M-6 | 48.9 | M-10 | 10.6 | — | — | 8,300 | 1.40 | 14.92 |

TABLE 2-2

| | | Each structural unit and Content | | | | | | | | | | | | |
|---|---|---|---|---|---|---|---|---|---|---|---|---|---|---|
| | | Structural | | Structural units | | Structural | | Structural | | Other | | | | Fluorine |
| | (A) | unit (I) | | (II) and (III) | | unit (IV) | | unit (VI) | | structural unit | | | | atom content |
| | Component | type | mol % | type | mol % | type | mol % | type | mol % | type | mol % | Mw | Mw/Mn | (% by mass) |
| Synthesis | a-1 | — | — | M-13 | 30.1 | M-2 | 69.9 | — | — | — | — | 7,000 | 1.41 | 10.20 |
| Example 1 | | | | | | | | | | | | | | |
| Synthesis | a-2 | — | — | M-12 | 41.1 | — | — | M-8 | 29.3 | — | — | 6,600 | 1.81 | 31.31 |
| Example 2 | | | | M-15 | 29.6 | | | | | | | | | |
| Synthesis | a-3 | — | — | M-12 | 40.9 | — | — | M-9 | 29.9 | — | — | 6,900 | 1.77 | 31.06 |
| Example 3 | | | | M-15 | 29.2 | | | | | | | | | |
| Synthesis | a-4 | — | — | M-12 | 41.3 | M-2 | 29.1 | M-8 | 29.6 | — | — | 6,900 | 1.88 | 19.94 |
| Example 4 | | | | | | | | | | | | | | |
| Synthesis | a-5 | — | — | — | — | — | — | — | — | M-22 | 100.0 | 6,900 | 1.55 | 19.78 |
| Example 5 | | | | | | | | | | | | | | |
| Synthesis | a-6 | — | — | — | — | M-2 | 31.2 | — | — | M-22 | 68.8 | 6,900 | 1.51 | 13.61 |
| Example 6 | | | | | | | | | | | | | | |
| Synthesis | a-7 | — | — | — | — | — | — | — | — | M-23 | 100.0 | 7,200 | 1.52 | 34.40 |
| Example 7 | | | | | | | | | | | | | | |
| Synthesis | a-8 | — | — | — | — | M-2 | 28.8 | — | — | M-23 | 71.2 | 7,100 | 1.53 | 27.15 |
| Example 8 | | | | | | | | | | | | | | |

### Synthesis of Polymer (C)

### Synthesis Examples 9 to 13

### Synthesis of Polymers (C-1) to (C-5)

Using the compounds as shown in Table 3, polymers (C-1) to (C-5) as the polymer (C) were prepared in a similar manner to Example 7. Physical property values of the polymers (C-1) to (C-5) are shown together in Table 3.

TABLE 3

| | | Monomer compound | | Structural | Physical property value | | |
|---|---|---|---|---|---|---|---|
| | | | amount | unit in the | | | fluorine |
| | (C) | | charged | polymer | | | atom content |
| | Polymer | type | mol % | content mol % | Mw | Mw/Mn | % by mass |
| Synthesis | C-1 | M-1 | 40 | 39.8 | 5,500 | 1.41 | 0.00 |
| Example 9 | | M-5 | 10 | 8.6 | | | |
| | | M-7 | 40 | 40.5 | | | |
| | | M-11 | 10 | 11.1 | | | |
| Synthesis | C-2 | M-2 | 20 | 21.1 | 5,500 | 1.43 | 0.00 |
| Example 10 | | M-4 | 30 | 28.5 | | | |
| | | M-5 | 10 | 8.8 | | | |
| | | M-7 | 40 | 41.6 | | | |
| Synthesis | C-3 | M-3 | 30 | 30.8 | 5,500 | 1.41 | 0.00 |
| Example 11 | | M-4 | 30 | 29.1 | | | |
| | | M-7 | 40 | 40.1 | | | |
| Synthesis | C-4 | M-1 | 30 | 30.5 | 6,000 | 1.39 | 0.00 |
| Example 12 | | M-4 | 10 | 9.5 | | | |
| | | M-5 | 10 | 8.8 | | | |
| | | M-7 | 30 | 31.1 | | | |
| | | M-10 | 20 | 20.1 | | | |
| Synthesis | C-5 | M-3 | 35 | 34.5 | 6,000 | 1.42 | 5.11 |
| Example 13 | | M-7 | 45 | 44.9 | | | |
| | | M-11 | 10 | 11.2 | | | |
| | | M-15 | 10 | 9.4 | | | |

### <Preparation of Radiation-Sensitive Resin Composition>

Each component for constituting the radiation-sensitive resin composition, other than the aforementioned polymer (A) and polymer (C), is shown below.

### (B) Acid Generating Agent: Structural Formulae Shown Below

(B-1): triphenylsulfonium nonafluoro-n-butanesulfonate

(B-2): 4-cyclohexylphenyldiphenylsulfonium nonafluoro-n-n butanesulfonate

(B-3): triphenylsulfonium 2-bicyclo[2.2.1]hept-2-yl-1,1,2,2-tetrafluoroethanesulfonate

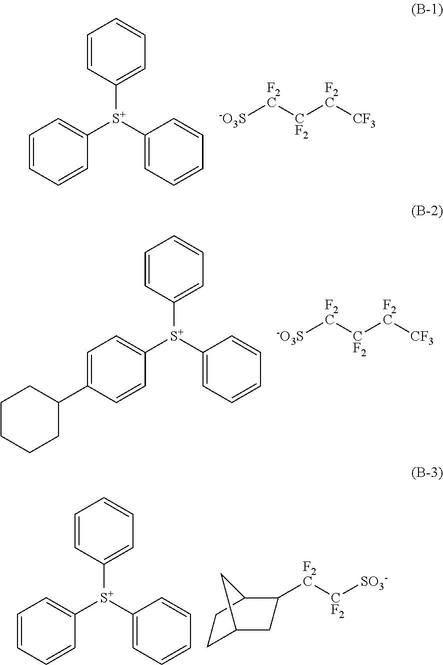

### (D) Acid Diffusion Control Agent: Structural Formulae Shown Below

(D-1): N-(t-butoxycarbonyl)-4-hydroxypiperidine

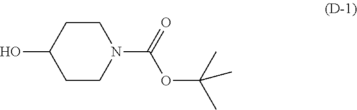

### (E) Solvent

(E-1): propylene glycol monomethyl ether acetate

(E-2): cyclohexanone

### (F) Additive (Uneven Distribution Accelerator)

(F-1): γ-butyrolactone

### Example 27

A radiation-sensitive resin composition was prepared by mixing 5 parts by mass of the polymer (A-1), 9.9 parts by mass of the acid generating agent (B-1), 100 parts by mass of the polymer (C-2) and 1.5 parts by mass of the acid diffusion control agent (D-1), 100 parts by mass of γ-butyrolactone as the additive (F), and 1,500 parts by mass of the solvent (E-1) and 650 parts by mass of the solvent (E-2).

### Examples 28 to 57, and Comparative Examples 1 to 8

Each radiation-sensitive resin composition was prepared in a similar manner to Example 27 except that the type and the amount of the component (A), the acid generating agent (B) and the polymer (C) were as shown in Tables 4-1, 4-2 and 4-3 in Example 27.

<Evaluation>

Resist coating films were formed with the radiation-sensitive resin compositions obtained in Examples 27 to 57 and Comparative Examples 1 to 8, and evaluations were made on the dynamic contact angle (advancing contact angle, receding contact angle) and defect prevention performance (number of bubble defects), according to each method described below. The results of the evaluations are shown in Tables 4-1, 4-2 and 4-3.

### [Measurement of Advancing Contact Angle and Receding Contact Angle]

Using each radiation-sensitive resin composition prepared as described above, a coating film was formed on a substrate (wafer). Thereafter, an advancing contact angle and a receding contact angle of the film thus formed were measured under a condition involving a room temperature of 23° C., a humidity of 45% and an ordinary pressure, using “DSA-10” manufactured by KRUS Electronics Ltd., according to the following procedure.

(Method of Measuring Advancing Contact Angle)

After the position of a wafer stage was adjusted, the wafer was placed on the stage, and water was introduced into a needle of “DSA-10”. After the position of the needle was accurately adjusted, water was discharged from the needle to form a water droplet of 10 μL on the wafer, and the needle was drawn once from the water droplet. Next, the needle was pulled down again to the accurately adjusted position, and thereafter water was discharged by the needle at a rate of 10 μL/min for 90 sec, and the contact angle was concomitantly measured every second (90 times in total). A mean value of the contact angles at 20 points was calculated after a contact angle was stably measured to determine the advancing contact angle (°).

(Method of Measuring Receding Contact Angle)

After the position of a wafer stage was adjusted, the wafer was placed on the stage, and water was introduced into a needle of “DSA-10”. After the position of the needle was accurately adjusted, water was discharged from the needle to form a water droplet of 25 μL on the wafer, and the needle was drawn once from the water droplet. Next, the needle was pulled down again to the accurately adjusted position, and thereafter the water droplet was aspirated by the needle at a rate of 10 μL/min for 90 sec, and the contact angle was concomitantly measured every second (90 times in total). A mean value of the contact angles at 20 points was calculated after a contact angle was stably measured to determine the receding contact angle (°).

On an 8 inch silicon wafer was formed a coating film having a film thickness of 110 nm with the radiation-sensitive resin composition described above, and soft baking (SB) was carried out at 120° C. for 60 sec. The advancing contact angle and the receding contact angle of thus resulting substrate were defined as “post SB advancing contact angle” and “post SB receding contact angle”, respectively.

On an 8 inch silicon wafer was formed a coating film having a film thickness of 110 nm with the radiation-sensitive resin composition described above, and soft baking (SB) was carried out at 120° C. for 60 sec. Thereafter, a development treatment was carried out with a 2.38% by mass aqueous tetramethylammonium hydroxide solution for 15 sec, followed by washing with water and drying. The advancing contact angle and the receding contact angle of thus resulting substrate were defined as “post development advancing contact angle” and “post development receding contact angle”, respectively.

### [Defect Prevention Performance (Measurement of Number of Generation of Bubble Defects)]

As a substrate, a 12 inch silicon wafer provided with an underlayer antireflective film having a film thickness of 105 nm (manufactured by Nissan Chemical Industries, Ltd., “ARC66”) formed on the surface thereof was used. It is to be noted that “CLEAN TRACK ACT12” manufactured by Tokyo Electron Limited was used for forming the antireflective film. Next, the radiation-sensitive resin composition prepared as described above was spin coated on the substrate with the aforementioned “CLEAN TRACK ACT12”, followed by prebaking (PB) carried out at 120° C. for 60 sec to form a photoresist film having a film thickness of 100 nm. The photoresist film was exposed through a mask having a line-and-space pattern (1L 1S) with a line width of 45 nm using an ArF excimer laser Immersion Scanner (manufactured by NIKON Corporation, “NIKON S610C”), with NA of 1.30, σ0/σI of 0.96/0.76, and a setting of Annular. In this procedure, pure water was disposed as a liquid immersion solvent between the superior surface of the resist and a lens of the liquid immersion lithography machine. Then, after PEB was conducted at 85° C. for 60 sec, development was carried out with a 2.38% by mass aqueous tetramethylammonium hydroxide solution at 23° C. for 60 sec, followed by washing with water and drying to form a positive type resist pattern. Thereafter, the number of defects on the line-and-space pattern (1L 1S) having a line width of 45 nm was measured using “KLA2810” manufactured by KLA-Tencor Corporation. Furthermore, defects determined with “KLA2810” were observed using a scanning electron microscope (manufactured by Hitachi High-Technologies Corporation, “S-9380”), and they were classified into those n deemed to be derived from the resist and those derived from a foreign unwanted substance and counted. Thus, the number of those classified to be derived from the ArF excimer laser liquid immersion lithography was defined as “number of bubble defects”.

TABLE 4-1

| | | | | | | | Advancing contact | | | Receding contact | | | |
|---|---|---|---|---|---|---|---|---|---|---|---|---|---|
| | | | (B) Acid | | | | angle (°) | | | angle (°) | | | |
| | (A) Component | | generating agent | | (C) Polymer | | | post | | | post | | Number |
| | | parts | | parts | | parts | post | develop- | differ- | post | develop- | differ- | of bubble |
| | type | by mass | type | by mass | type | by mass | SB | ment | ence | SB | ment | ence | defects |
| Example 27 | A-1 | 5 | B-1 | 9.9 | C-2 | 100 | 102 | 78 | 24 | 82 | 42 | 40 | 0 |
| Example 28 | A-2 | 5 | B-1 | 9.9 | C-1 | 100 | 93 | 80 | 13 | 71 | 49 | 22 | 0 |
| Example 29 | A-2 | 5 | B-1 | 9.9 | C-2 | 100 | 94 | 82 | 12 | 73 | 51 | 22 | 0 |
| Example 30 | A-3 | 3 | B-1 | 9.9 | C-2 | 100 | 98 | 85 | 13 | 76 | 50 | 26 | 0 |
| Example 31 | A-4 | 3 | B-1 | 9.9 | C-2 | 100 | 95 | 80 | 15 | 78 | 50 | 28 | 0 |
| Example 32 | A-5 | 3 | B-1 | 9.9 | C-2 | 100 | 91 | 77 | 14 | 71 | 50 | 21 | 0 |
| Example 33 | A-6 | 3 | B-1 | 9.9 | C-2 | 100 | 92 | 79 | 13 | 71 | 52 | 19 | 0 |
| Example 34 | A-7 | 3 | B-1 | 9.9 | C-2 | 100 | 93 | 81 | 12 | 82 | 44 | 38 | 0 |
| Example 35 | A-8 | 3 | B-1 | 9.9 | C-2 | 100 | 79 | 67 | 12 | 66 | 36 | 30 | 0 |
| Example 36 | A-9 | 3 | B-1 | 9.9 | C-2 | 100 | 105 | 85 | 20 | 88 | 52 | 36 | 0 |
| Example 37 | A-10 | 3 | B-1 | 9.9 | C-2 | 100 | 103 | 92 | 11 | 85 | 46 | 39 | 0 |
| Example 38 | A-11 | 1 | B-1 | 9.9 | C-2 | 100 | 99 | 89 | 10 | 83 | 45 | 38 | 0 |
| Example 39 | A-7 | 1 | B-1 | 9.9 | C-2 | 100 | 88 | 76 | 12 | 79 | 51 | 28 | 0 |
| Example 40 | A-7 | 2 | B-1 | 9.9 | C-2 | 100 | 90 | 78 | 12 | 81 | 48 | 33 | 0 |
| Example 41 | A-7 | 5 | B-1 | 9.9 | C-2 | 100 | 92 | 79 | 13 | 85 | 43 | 42 | 0 |

TABLE 4-2

| | | | | | | | Advancing contact | | | Receding contact | | | |
|---|---|---|---|---|---|---|---|---|---|---|---|---|---|
| | | | (B) Acid | | | | angle (°) | | | angle (°) | | | |
| | (A) Component | | generating agent | | (C) Polymer | | | post | | | post | | Number |
| | | parts | | parts | | parts | post | develop- | differ- | post | develop- | differ- | of bubble |
| | type | by mass | type | by mass | type | by mass | SB | ment | ence | SB | ment | ence | defects |
| Example 42 | A-7 | 2 | B-2 | 11.4 | C-2 | 100 | 90 | 75 | 15 | 81 | 46 | 35 | 0 |
| Example 43 | A-7 | 2 | B-3 | 9.5 | C-2 | 100 | 89 | 76 | 13 | 78 | 50 | 28 | 0 |
| Example 44 | A-7 | 2 | B-2 | 11.4 | C-3 | 100 | 91 | 74 | 17 | 80 | 44 | 36 | 0 |
| Example 45 | A-7 | 3 | B-2 | 11.4 | C-4 | 100 | 92 | 77 | 15 | 82 | 45 | 37 | 0 |
| Example 46 | A-7 | 3 | B-2 | 11.4 | C-5 | 100 | 91 | 73 | 18 | 80 | 40 | 40 | 0 |
| Example 47 | A-11 | 3 | B-1 | 9.9 | C-2 | 100 | 90 | 79 | 11 | 75 | 53 | 22 | 0 |
| Example 48 | A-12 | 3 | B-2 | 11.4 | C-4 | 100 | 92 | 82 | 10 | 83 | 41 | 42 | 0 |
| Example 49 | A-13 | 3 | B-2 | 11.4 | C-4 | 100 | 91 | 80 | 11 | 81 | 41 | 40 | 0 |
| Example 50 | A-14 | 3 | B-2 | 11.4 | C-4 | 100 | 91 | 80 | 11 | 81 | 45 | 36 | 0 |
| Example 51 | A-15 | 3 | B-2 | 11.4 | C-4 | 100 | 89 | 78 | 11 | 76 | 52 | 24 | 0 |
| Example 52 | A-16 | 3 | B-2 | 11.4 | C-4 | 100 | 87 | 74 | 13 | 76 | 48 | 28 | 0 |
| Example 53 | A-17 | 3 | B-2 | 11.4 | C-4 | 100 | 88 | 74 | 14 | 78 | 44 | 34 | 0 |
| Example 54 | A-18 | 3 | B-3 | 9.5 | C-1 | 100 | 99 | 57 | 42 | 84 | 15 | 69 | 0 |
| Example 55 | A-19 | 3 | B-3 | 9.5 | C-1 | 100 | 94 | 55 | 39 | 79 | 35 | 44 | 0 |
| Example 56 | A-20 | 3 | B-3 | 9.5 | C-1 | 100 | 91 | 61 | 30 | 79 | 34 | 45 | 0 |
| Example 57 | A-21 | 3 | B-3 | 9.5 | C-1 | 100 | 92 | 59 | 33 | 75 | 33 | 42 | 0 |

TABLE 4-3

| | | | | | | | Advancing contact | | | Receding contact | | | |
|---|---|---|---|---|---|---|---|---|---|---|---|---|---|
| | | | (B) Acid | | | | angle (°) | | | angle (°) | | | |
| | (A) Component | | generating agent | | (C) Polymer | | | post | | | post | | Number |
| | | parts | | parts | | parts | post | develop- | differ- | post | develop- | differ- | of bubble |
| | type | by mass | type | by mass | type | by mass | SB | ment | ence | SB | ment | ence | defects |
| Comparative | a-1 | 3 | B-1 | 9.9 | C-2 | 100 | 91 | 91 | 0 | 78 | 77 | 1 | 0 |
| Example 1 | | | | | | | | | | | | | |
| Comparative | a-2 | 5 | B-1 | 9.9 | C-2 | 100 | 84 | 83 | 1 | 70 | 60 | 10 | 0 |
| Example 2 | | | | | | | | | | | | | |
| Comparative | a-3 | 5 | B-1 | 9.9 | C-2 | 100 | 79 | 76 | 3 | 64 | 58 | 6 | 0 |
| Example 3 | | | | | | | | | | | | | |
| Comparative | a-4 | 5 | B-1 | 9.9 | C-2 | 100 | 86 | 80 | 6 | 76 | 55 | 21 | 0 |
| Example 4 | | | | | | | | | | | | | |
| Comparative | a-5 | 5 | B-1 | 9.9 | C-2 | 100 | 117 | 88 | 29 | 91 | 41 | 50 | 122 |
| Example 5 | | | | | | | | | | | | | |
| Comparative | a-6 | 5 | B-1 | 9.9 | C-2 | 100 | 87 | 80 | 7 | 72 | 60 | 12 | 132 |
| Example 6 | | | | | | | | | | | | | |
| Comparative | a-7 | 5 | B-1 | 9.9 | C-2 | 100 | 91 | 84 | 7 | 78 | 52 | 26 | 0 |
| Example 7 | | | | | | | | | | | | | |
| Comparative | a-8 | 5 | B-1 | 9.9 | C-2 | 100 | 86 | 79 | 7 | 76 | 52 | 24 | 0 |
| Example 8 | | | | | | | | | | | | | |

From the results shown in Tables 4-1, 4-2 and 4-3, it was ascertained that any of the resist films formed using the radiation-sensitive resin compositions of Examples 27 to 57 containing the polymer (A) according to the embodiment of the present invention has a greater post SB receding contact angle with respect to water, as compared with the resist film formed using the radiation-sensitive resin compositions of Comparative Examples 1 to 8 not containing the polymer (A). Therefore, it is proven that the hydrophobicity of a resist film increases due to containing the polymer (A). Accordingly, improvement of both the effects of scan following capability and reduction of elution during the liquid immersion scanning exposure can be expected.

In addition, from the results shown in Tables 4-1, 4-2 and 4-3, it was confirmed that any of the resists formed using the radiation-sensitive resin compositions of Examples 27 to 57 exhibited significantly decreased post development dynamic contact angle with respect to water, both the advancing contact angle and the receding contact angle, as compared with the contact angle before the development, and that in particular, the receding contact angle remarkably significantly decreased. Thus, due to containing the polymer (A), it is expected that spreadability of a developer and a rinse liquid can be improved and also expected that an effect of decreasing defects derived from liquid immersion can be achieved. Moreover, according to the radiation-sensitive resin compositions of Examples 54 to 57 in which the polymer (A) was used, wherein n in the above formula (1) representing the structural unit (I) is 2, it was also shown that a decrease of both the advancing contact angle and the receding contact angle of post development as compared to that before the development can be further enhanced. Accordingly, it is expected that the scan following capability, an effect of the reduction of elution, as well as spreadability of a developer and a rinse liquid, and the like can be further improved.

On the other hand, Comparative Examples 6 and 7 based on the polymer having a fluorine-containing group with a large number of fluorine atoms exhibited enormous generation of bubble defects. It was indicated that according to the radiation-sensitive resin composition of the embodiment of the present invention, such generation of bubble defects is sufficiently inhibited.

The radiation-sensitive resin composition of the embodiment of the present invention can be suitably used as a chemically amplified resist for use in producing a semiconductor device, particularly a resist for liquid immersion lithography, and the like. More specifically, the radiation-sensitive resin composition, the method for forming a resist pattern using the composition, the polymer suited as a constitutive component of the composition, and the compound suited as a monomer of the polymer are suitably used as a resist composition for liquid immersion lithography.

Obviously, numerous modifications and variations of the present invention are possible in light of the above teachings. It is therefore to be understood that within the scope of the appended claims, the invention may be practiced otherwise than as specifically described herein.

1. A radiation-sensitive resin composition comprising:
a first polymer having a structural unit represented by a following formula (1); and
a radiation-sensitive acid generator, 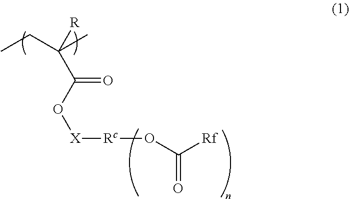
wherein, in the formula (1),
R represents a hydrogen atom, a methyl group or a trifluoromethyl group;
X represents a single bond or a bivalent linking group;
R^{C} represents an aliphatic cyclic hydrocarbon group having a valency of (n+1) and having 3 to 30 carbon atoms, wherein the aliphatic cyclic hydrocarbon group represented by R^{C} is unsubstituted or a part or all of hydrogen atoms included in the aliphatic cyclic hydrocarbon group represented by R^{C} are each substituted;
Rf represents a monovalent chain hydrocarbon group having 1 to 30 carbon atoms and having 1 to 10 fluorine atoms, or a monovalent aliphatic cyclic hydrocarbon group having 3 to 30 carbon atoms and having 1 to 10 fluorine atoms; and
n is an integer of 1 to 3, wherein in a case where n is 2 or 3, Rfs present in plural number are a same or different.

2. The radiation-sensitive resin composition according to claim 1, wherein R^{C} in the formula (1) represents an aliphatic polycyclic hydrocarbon group having a valency of (n+1) and having 4 to 30 carbon atoms.

3. The radiation-sensitive resin composition according to claim 1, wherein the structural unit represented by the formula (1) is a structural unit represented by a following formula (1-1): 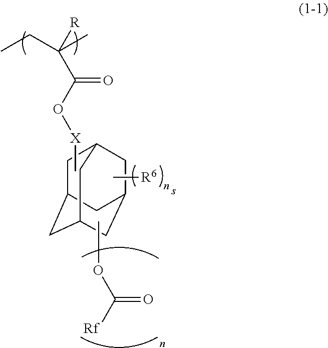
wherein, in the formula (1-1),
each of R, X, Rf and n is as defined in the formula (1);
R^{S} represents —R^{P1}, —R^{P2}—O—R^{P1}, —R^{P2}—CO—R^{P1}, —R^{P2}—CO—OR^{P}', —R^{P2}—O—CO—R^{P1}, —R^{P2}—OH, —R^{P2}—CN or —R^{P2}—COOH;
R^{P1} represents a monovalent chain saturated hydrocarbon group having 1 to 10 carbon atoms, a monovalent aliphatic cyclic saturated hydrocarbon group having 3 to 20 carbon atoms or a monovalent aromatic hydrocarbon group having 6 to 30 carbon atoms, wherein R^{P1} is unsubstituted or a part or all of hydrogen atoms included in R^{P1} are each substituted with a fluorine atom;
R^{P2} represents a single bond, a bivalent chain saturated hydrocarbon group having 1 to 10 carbon atoms, a bivalent aliphatic cyclic saturated hydrocarbon group having 3 to 20 carbon atoms or a bivalent aromatic hydrocarbon group having 6 to 30 carbon atoms, wherein R^{P2} is unsubstituted or a part or all of hydrogen atoms included in R^{P2} are each substituted with a fluorine atom; and
n_{S} is an integer of 0 to 3.

4. The radiation-sensitive resin composition according to claim 3, wherein the structural unit represented by the formula (1-1) is a structural unit represented by a following formula (1-1a), a structural unit represented by a following formula (1-1b), a structural unit represented by a following formula (1-1c), or a combination thereof: 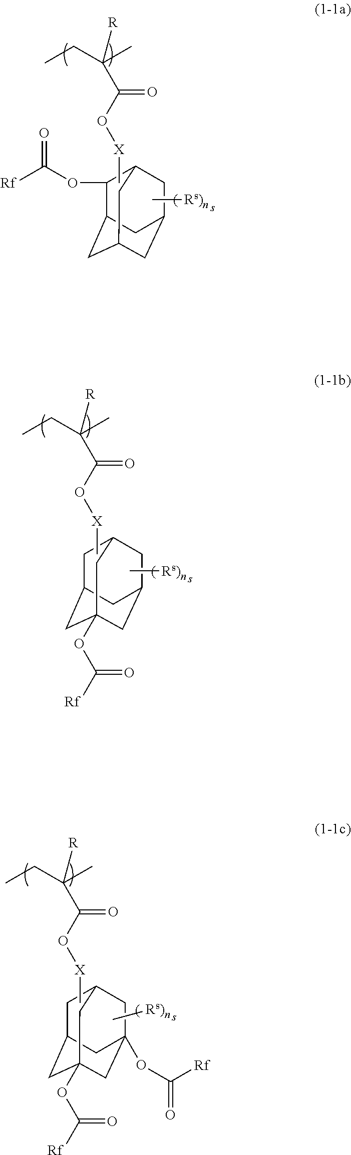
wherein, in the formulae (1-1a), (1-1b) and (1-1c), each of R, X, Rf, R^{S} and n_{S} is as defined in the formula (1-1).

5. The radiation-sensitive resin composition according to claim 1 further comprising:
a second polymer having fluorine atoms and an acid-dissociable group, a content of the fluorine atoms included in the second polymer being less than a content of fluorine atoms included in the first polymer.

6. The radiation-sensitive resin composition according to claim 1, wherein the first polymer further has a structural unit represented by a following formula (2) and a structural unit represented by a following formula (3): 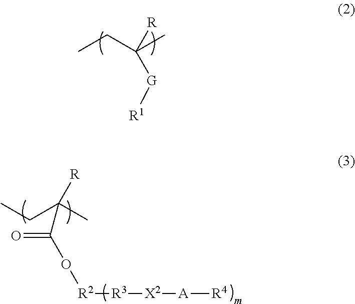
wherein, in the formulae (2) and (3),
R represents a hydrogen atom, a methyl group or a trifluoromethyl group,
wherein in the formula (2),
G represents a single bond, an oxygen atom, a sulfur atom, —CO—O—, —SO₂—O—NH—, —CO—NH— or —O—CO—NH—; and
R¹ represents a monovalent chain hydrocarbon group having 1 to 6 carbon atoms and having at least one fluorine atom or a monovalent aliphatic cyclic hydrocarbon group having 4 to 20 carbon atoms and having at least one fluorine atom, and wherein, in the formula (3),
R² represents a hydrocarbon group having 1 to 20 carbon atoms, having a valency of (m+1), and optionally having a structure in which an oxygen atom, a sulfur atom, —NR′—, carbonyl group, —CO—O— or —CO—NH— is bonded to an end of R² on a side of R³;
R′ represents a hydrogen atom or a monovalent organic group;
R³ represents a single bond, a bivalent chain hydrocarbon group having 1 to 10 carbon atoms or a bivalent aliphatic cyclic hydrocarbon group having 4 to 20 carbon atoms;
X² represents a bivalent chain hydrocarbon group having 1 to 20 carbon atoms and having at least one fluorine atom;
A represents an oxygen atom, —NR″—, —CO—O—* or —SO₂—O—*;
wherein R″ represents a hydrogen atom or a monovalent organic group, and * denotes a binding site that binds to R⁴;
R⁴ represents a hydrogen atom or a monovalent organic group; and
m is an integer of 1 to 3, wherein in a case where m is 2 or 3, each of R^{a}s, X²s, As and R⁴s present in plural number are a same or different.

7. A method for forming a resist pattern, comprising:
providing the radiation-sensitive resin composition according to claim 1 on a substrate to form a photoresist film;
disposing a liquid for immersion lithography on the photoresist film;
exposing the photoresist film through the liquid for immersion lithography; and
developing the exposed photoresist film to form a resist pattern.

8. A polymer comprising:
a structural unit represented by a following formula (1): 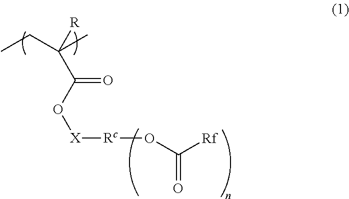
wherein, in the formula (1),
R represents a hydrogen atom, a methyl group or a trifluoromethyl group;
X represents a single bond or a bivalent linking group;
R^{C} represents an aliphatic cyclic hydrocarbon group having a valency of (n+1) and having 3 to 30 carbon atoms, wherein the aliphatic cyclic hydrocarbon group represented by R^{C} is unsubstituted or a part or all of hydrogen atoms included in the aliphatic cyclic hydrocarbon group represented by R^{C} are each substituted;
Rf represents a monovalent chain hydrocarbon group having 1 to 30 carbon atoms and having 1 to 10 fluorine atoms, or a monovalent aliphatic cyclic hydrocarbon group having 3 to 30 carbon atoms and having 1 to 10 fluorine atoms; and
n is an integer of 1 to 3, wherein in a case where n is 2 or 3, Rfs present in plural number are a same or different.

9. The polymer according to claim 8, further comprising:
a structural unit represented by a following formula (2); and
a structural unit represented by a following formula (3): 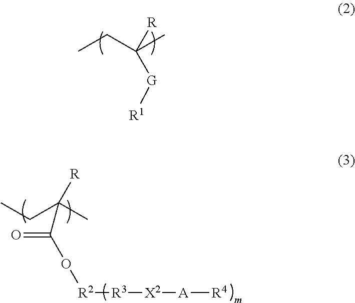
wherein, in the formulae (2) and (3),
R represents a hydrogen atom, a methyl group or a trifluoromethyl group,
wherein in the formula (2),
G represents a single bond, an oxygen atom, a sulfur atom, —CO—O—, —SO₂—O—NH—, —CO—NH— or —O—CO—NH—; and
R¹ represents a monovalent chain hydrocarbon group having 1 to 6 carbon atoms and having at least one fluorine atom or a monovalent aliphatic cyclic hydrocarbon group having 4 to 20 carbon atoms and having at least one fluorine atom, and wherein, in the formula (3),
R² represents a hydrocarbon group having 1 to 20 carbon atoms, having a valency of (m+1), and optionally having a structure in which an oxygen atom, a sulfur atom, —NR′—, a carbonyl group, —CO—O— or —CO—NH— is bonded to an end of R² on a side of R³;
R′ represents a hydrogen atom or a monovalent organic group;
R³ represents a single bond, a bivalent chain hydrocarbon group having 1 to 10 carbon atoms or a bivalent aliphatic cyclic hydrocarbon group having 4 to 20 carbon atoms;
X² represents a bivalent chain hydrocarbon group having 1 to 20 carbon atoms and having at least one fluorine atom;
A represents an oxygen atom, —NR″—, —CO—O—* or —SO₂—O—*, wherein R″ represents a hydrogen atom or a monovalent organic group, and * denotes a binding site that binds to R⁴;
R⁴ represents a hydrogen atom or a monovalent organic group; and
m is an integer of 1 to 3, wherein in a case where m is 2 or 3, each of R^{a}s, X²s, As and R⁴s present in plural number are a same or different.

10. A compound represented by a following formula (i): 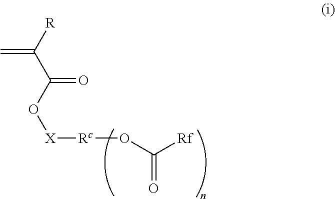
wherein, in the formula (i),
R represents a hydrogen atom, a methyl group or a trifluoromethyl group;
X represents a single bond or a bivalent linking group;
R^{C} represents an aliphatic cyclic hydrocarbon group having a valency of (n+1) and having 3 to 30 carbon atoms, wherein the aliphatic cyclic hydrocarbon group represented by R^{C} is unsubstituted or a part or all of hydrogen atoms included in the aliphatic cyclic hydrocarbon group represented by R^{C} are each substituted;
Rf represents a monovalent chain hydrocarbon group having 1 to 30 carbon atoms and having 1 to 10 fluorine atoms, or a monovalent aliphatic cyclic hydrocarbon group having 3 to 30 carbon atoms and having 1 to 10 fluorine atoms; and
n is an integer of 1 to 3, wherein in a case where n is 2 or 3, Rfs present in plural number are a same or different.
